(19) United States
(12) Patent Application Publication
(10) Pub. No.: US 2026/0308662 A1
(43) Pub. Date: Oct. 8, 2026

(54) EPIGENOMIC ANALYSIS OF FORMALIN-FIXED PARAFFIN-EMBEDDED SAMPLES

(71) Applicant: Fred Hutchinson Cancer Center, Seattle, WA (US)

(72) Inventor: Steve Henikoff, Seattle, WA (US)

(21) Appl. No.: 19/484,738

(22) PCT Filed: May 31, 2024
(86) PCT No.: PCT/US2024/031983

§ 371 Date: Nov. 14, 2025

Related U.S. Application Data
(60) Provisional application No. 63/505,964, filed on Jun. 2, 2023.

(51) Int. Cl. C12Q 1/6804; C12Q 1/6886

(52) U.S. Cl. CPC C12Q 1/6804; C12Q 1/6886; C12Q 2600/156

(57) ABSTRACT

Disclosed herein are improved in situ methods for mapping the location of a protein on chromatin and DNA-based in situ methods for measuring transcription in a cell from a formalin-fixed paraffin-embedded (FFPE) sample. The methods can include steps of treating the FFPE sample to remove paraffin; permeabilizing the sample; contacting the sample with a first affinity reagent that specifically binds to a targeted chromatin protein or a protein involved in transcription regulation, wherein the first affinity reagent is coupled to a transposome comprising at least one transposase and a transposon; activating the at least one transposase under low ionic conditions, thereby cleaving and tagging chromatin DNA; excising the tagged DNA segment associated with the targeted chromatin protein or protein involved in transcription regulation; and determining the nucleotide sequence of the excised tagged DNA segment, thereby mapping the genomic location of the targeted protein on chromatin or transcriptional activity on chromatin.

### STATEMENT OF PRIORITY

This application claims the benefit of U.S. Provisional Application Ser. No. 63/505,964, filed Jun. 2, 2023, the entire contents of which are incorporated by reference herein.

### FIELD OF INVENTION

The invention relates to assays for detecting and/or quantitating sites of DNA accessibility in chromatin in formalin-fixed paraffin-embedded (FFPE) samples. The invention further relates to methods of using the assay for epigenomic profiling of FFPE samples.

### STATEMENT REGARDING ELECTRONIC FILING OF A SEQUENCE LISTING

A Sequence Listing in XML format, entitled 1426-46WO_ST26.xml, 97 bytes in size, generated on May 30, 2024 and filed herewith, is hereby incorporated by reference in its entirety for its disclosures.

### BACKGROUND

For more than a century, formalin-fixed paraffin-embedded (FFPE) sample preparation has been the preferred method for long-term preservation of biological material. However, the use of FFPE samples for epigenomic studies has been difficult because of chromatin damage from long exposure to high concentrations of formaldehyde.

The resurrection of biological samples from long-term storage has become a major enterprise in recent years, as it makes possible the application of modern sequencing-based genomic methodologies to archived cytological samples for ongoing and retrospective studies (Saqi, A. The state of cell blocks and ancillary testing: past, present, and future. Arch. Pathol. Lab Med. 140, 1318-1322 (2016)). The preferred method for sample preservation has been fixation in formalin (~4% formaldehyde) for a few days followed by dehydration and embedding in paraffin. FFPE sample preservation has been in use for over a century, with billions of cell blocks accumulated thus far, and no end in sight (Blow, N. Tissue preparation: tissue issues. Nature 448, 959-963 (2007)). Most genomic studies using FFPE samples have applied whole genome sequencing to identify mutations and aneuploidies, or whole exome sequencing to identify tissue-specific differences. However, chromatin profiling has the potential of identifying causal regulatory element changes that drive disease. The prospect of applying chromatin profiling to distinguish regulatory element changes is especially attractive for translational cancer research, insofar as misregulation of promoters and enhancers in cancer can provide diagnostic information and may be targeted for therapy (Armstrong, S. A., Henikoff, S. & Vakoc, C. R. Chromatin Deregulation in Cancer (Cold Spring Harbor Press, 2017)). However, there has been limited progress in applying chromatin profiling techniques to FFPEs (Amatori, S. & Fanelli, M. The current state of Chromatin Immunoprecipitation (ChIP) from FFPE tissues. Int J. Mol. Sci. 23, 1103 (2022)). Although several methods have been developed for chromatin immunoprecipitation with sequencing (ChIP-seq) using FFPEs (See, e.g., Kaneko, S. et al. Genome-wide chromatin analysis of FFPE tissues using a dual-arm robot with clinical potential. Cancers (Basel) 13, 2126 (2021); Font-Tello, A. et al. FiTAc-seq: fixed-tissue ChIP-seq for H3K27ac profiling and super-enhancer analysis of FFPE tissues. Nat. Protoc. 15, 2503-2518 (2020); Amatori, S. et al. Epigenomic profiling of archived FFPE tissues by enhanced PAT-ChIP (EPAT-ChIP) technology. Clin. Epigenetics 10, 143 (2018); Fanelli, M. et al. Pathology tissue-chromatin immunoprecipitation, coupled with high-throughput sequencing, allows the epigenetic profiling of patient samples. Proc. Natl Acad. Sci. USA 107, 21535-21540 (2010); Cejas, P. et al. Chromatin immunoprecipitation from fixed clinical tissues reveals tumor-specific enhancer profiles. Nat. Med. 22, 685-691 (2016); Zhong, J. et al. Enhanced and controlled chromatin extraction from FFPE tissues and the application to ChIP-seq. BMC Genom. 20, 249 (2019)), ChIP-seq is not well-suited for small amounts of material that are typically available from patient samples. Furthermore, solubilization of such heavily cross-linked material is extremely challenging, requiring strong ionic detergents and/or proteases in addition to controlled sonication or micrococcal nuclease (MNase) digestion treatments.

Alternatives to ChIP-seq for chromatin profiling include ATAC-seq (Buenrostro, J. D., Giresi, P. G., Zaba, L. C., Chang, H. Y. & Greenleaf, W. J. Transposition of native chromatin for fast and sensitive epigenomic profiling of open chromatin, DNA-binding proteins and nucleosome position. Nat. Methods 10, 1213-1218 (2013)), DNase-seq (Jin, W. et al. Genome-wide detection of DNase I hypersensitive sites in single cells and FFPE tissue samples. Nature 528, 142-146 (2015)), NicE-seq (Vishnu et al. One-pot universal NicE-seq: all enzymatic downstream processing of 4% formaldehyde crosslinked cells for chromatin accessibility genomics. Epigenetics Chromatin 14, 53 (2021)), FAIRE ^{(see, e.g.,} Marcel, S. S. et al. Genome-wide cancer-specific chromatin accessibility patterns derived from archival processed xenograft tumors. Genome Res. 31, 2327-2339 (2021)) and enzyme-tethering methods such as CUT&RUN (Skene, P. J. & Henikoff, S. An efficient targeted nuclease strategy for high-resolution mapping of DNA binding sites. Elife 6, e21856 (2017)) and CUT&Tag (Kaya-Okur, H. S. et al. CUT&Tag for efficient epigenomic profiling of small samples and single cells. Nat. Commun. 10, 1930 (2019)). Modifications to the standard ATAC-seq protocol were required to make it suitable for FFPEs, including nuclei isolation following enzymatic tissue disruption and in vitro transcription with T7 RNA polymerase (Yadav, R. P., Polavarapu, V. K., Xing, P. & Chen, X. FFPE-ATAC: A highly sensitive method for profiling chromatin accessibility in formalin-fixed paraffin-embedded samples. Curr. Protoc. 2, e535 (2022); Zhang, H. et al. Profiling chromatin accessibility in formalin-fixed paraffin-embedded samples. Genome Res. 32, 150-161 (2022)). The same group also similarly modified CUT&Tag and included an epitope retrieval step using ionic detergents and elevated temperatures, which they termed FFPE tissue with Antibody-guided Chromatin Tagmentation with sequencing (FACT-seq) (Zhao, L. et al. FACT-seq; profiling histone modifications in formalin-fixed paraffin-embedded samples with low cell numbers. Nucleic Acids Res. 49, e125 (2021); Zhao, L., Polavarapu, V. K., Yadav, R. P., Xing, P. & Chen, X. A highly sensitive method to efficiently profile the histone modifications of FFPE samples. Bio Protoc. 12, c4418 (2022)). However, FACT-seq is a 5-day protocol even before sequencing, and the many extra steps required relative to CUT&Tag have raised concerns about experimental variability (Amatori, 2022). Additionally, both the FFPE-ATAC and FACT-seq methods require lengthy digestion with collagenases and hyaluronidases followed by needle extraction and straining liberated nuclei for processing.

The quantity and quality of extracted DNA and RNA from FFPE blocks differs widely, with DNA less likely to suffer from degradation than RNA in the samples. See, e.g., Chougule, et al., Diagnostics 2022, 12, 1291.

While RNA sequencing (RNA-seq) enables unique insights into clinical samples that can potentially lead to mechanistic understanding of the basis of various diseases as well as resistance and/or susceptibility mechanisms, FFPE tissues, which represent the most common method for preserving tissue morphology in clinical specimens, are not the best sources for gene expression profiling analysis using RNA. Exposure of tissue to ~4% formaldehyde for days badly damages RNA and DNA and causes cross-links to form between tightly bound proteins and nucleic acids. The RNA obtained from such samples is often badly degraded, fragmented, and chemically modified, which leads to suboptimal sequencing while DNA is better preserved. See, e.g., Chougule et al., Comprehensive Development and Implementation of Good Laboratory Practice for NGS Based Targeted Panel on Solid Tumor FFPE Tissues in Diagnostics. Diagnostics 12, 1291 (2002). Moreover, while hypertranscription in cancer has been frequently documented in studies based on RNA-seq (Zatzman M, Fuligni F, Ripsman R, Suwal T, Comitani F, Edward L M, et al. Widespread hypertranscription in aggressive human cancers. Sci Adv. 2022; 8 (47): eabn0238; Cao S, Wang J R, Ji S, Yang P, Dai Y, Guo S, et al. Estimation of tumor cell total mRNA expression in 15 cancer types predicts disease progression. Nat Biotechnol. 2022; 40 (11): 1624-33), these indirect methods have limitations owing to variable processing of mRNAs, to the low level of mRNAs encoding critical regulatory proteins, and to the need for accurate calibration to genomic DNA abundance. Crucially, none of the methods that have been applied to measure hypertranscription in cancer are suitable for FFPEs, which have long been standard for archival storage of tissue samples (Blow N. Tissue preparation: Tissue issues. Nature. 2007; 448 (7156): 959-63). An advantage over the art would be the ability to determine RNA transcription from a DNA.

Accordingly, despite the advances in the art, there remains a need for facile and accurate analyses to identify active regulatory elements across a genome in FFPE samples. It would be an advance in the art to provide an affordable and rapid epigenomic profiling of archived biological samples for biomarker identification, clinical applications and retrospective studies. This disclosure addresses these and related needs.

### SUMMARY

Embodiments of the present invention are based, in part, on the development of assays for chromatin profiling of FFPE samples, allowing for simultaneous chromatin profiling and accessibility mapping in FFPE samples with improved signal to noise at a low cost and improved speed compared to the current state of the art assays. Embodiments of the present invention are also based, in part, on assays using RNA polymerase II (RNAPII) profiling in FFPE samples to map the transcriptional machinery itself directly on the DNA regulatory elements to obtain direct measurements of transcription activity, including nascent transcription.

In an aspect, an in situ method of mapping the location of a protein on chromatin in a cell from a FFPE sample is provided, comprising treating the FFPE sample to remove the paraffin; permeabilizing the sample; contacting the sample with a first affinity reagent that specifically binds to a targeted chromatin protein, wherein the first affinity reagent is coupled to at least one transposome comprising: at least one transposase; and a transposon comprising: a first DNA molecule comprising a first transposase recognition site; and a second DNA molecule comprising a second transposase recognition site; activating the at least one transposase under low ionic conditions, thereby cleaving and tagging chromatin DNA with the first and second DNA molecules; excising the tagged DNA segment associated with the targeted chromatin protein; and determining the nucleotide sequence of the excised tagged DNA segment, thereby mapping the genomic location of the targeted protein on chromatin.

In another aspect, a DNA-based in situ method for measuring transcription in a cell from a FFPE sample is provided, comprising: treating the FFPE sample to remove the paraffin; permeabilizing the sample; contacting the sample with a first affinity reagent that specifically binds to a protein involved in transcription regulation, wherein the first affinity reagent is coupled to at least one transposome comprising: at least one transposase; and a transposon comprising: a first DNA molecule comprising a first transposase recognition site; and a second DNA molecule comprising a second transposase recognition site; activating the at least one transposase under low ionic conditions, thereby cleaving and tagging chromatin DNA with the first and second DNA molecules; excising the tagged DNA segment associated with the protein involved in transcription regulation; and determining the nucleotide sequence of the excised tagged DNA segment, thereby mapping transcriptional activity on chromatin.

In another aspect, methods of monitoring a disease or disorder are provided, comprising performing a method as described herein on samples obtained at two or more points in time from the same subject, and comparing an amount and/or the genomic location of the targeted protein on chromatin and/or the transcriptional activity on chromatin in each sample to a reference and/or to each other.

In another aspect, the disclosure provides a method of diagnosing a disease or disorder in a subject, comprising performing a method as described herein on a sample from the subject, and diagnosing the subject as having the disease or disorder based on an amount and/or the genomic location of the targeted protein on chromatin and/or the transcriptional activity on chromatin to thereby diagnose the subject as having the disease or disorder.

In a further aspect, a method of prognosing a disease or disorder in a subject is provided, the method comprising performing a method as described herein on a sample from the subject, and prognosing the disease or disorder in the subject based on the amount and/or genomic location of the targeted protein on chromatin and/or the transcriptional activity on chromatin.

In an aspect, the disclosure provides a method of detecting hypertranscription in a sample, comprising performing a method as described herein, wherein an increased amount of transcriptional activity on chromatin thereby detects hypertranscription in the sample.

In another aspect, the disclosure provides a method of quantifying increases or decreases in RNAPII over a plurality of loci, comprising performing a method as described herein, wherein the first affinity reagent is an affinity reagent specific for RNAPII, e.g., a phosphoform of the C-terminal domain of RNAPII, such as RNAPII-Ser2, RNAPII-Ser5, RNAPII-Ser7, RNAPII-Ser2/5, or RNAPII-Ser5/7, and further comprising comparing the results to a control reference.

In an aspect, the disclosure provides a method of detecting presence of a protein of interest on chromatin, comprising performing a method as described herein, wherein the first affinity reagent that specifically binds to the targeted chromatin protein is specific for the protein of interest to thereby detect the presence of the protein of interest on chromatin.

In an additional aspect, the disclosure provides a method of detecting an amount of a protein of interest on chromatin, comprising performing a method as described herein, wherein the first affinity reagent that specifically binds to the targeted chromatin protein is specific for the protein of interest to thereby detect the amount of the protein of interest on chromatin.

In a further aspect, the disclosure provides a method of detecting an epigenetic modification on a protein, comprising performing a method as described herein, to determine the presence of the epigenetic modification on the protein.

In an aspect, the disclosure provides a composition comprising a deparaffinized and permeabilized FFPE sample containing an RNAPII-specific affinity reagent that is linked directly or indirectly to a transposome in low ionic conditions.

In another aspect, the disclosure provides a composition comprising a deparaffinized and permeabilized FFPE sample containing a chromatin protein specific affinity reagent that is linked directly or indirectly to a transposome in low ionic conditions.

In a further aspect, the disclosure provides a kit comprising two or more reagents selected from a RNAPII-specific affinity reagent, one or more chromatin protein-specific affinity reagent, a SDS solution, a Triton® X-100 (octyl phenol ethoxylate) solution, a transposase solution, a tagmentation buffer, a cross-linking reversal solution, and amine-functionalized magnetic beads.

These and other aspects of the invention are set forth in more detail in the description of the invention below.

### BRIEF DESCRIPTION OF THE DRAWINGS

FIGS. 1A-1C. High data quality from CUT&Tag-direct for whole cells. 1A) A comparison of H3K4me3 CUT&Tag tracks for K562 cells (tracks 2-6) at a representative 100-kb region of housekeeping genes, showing group-autoscaled profiles for 4 million mapped fragments from each sample. 1B-1C) Number of Peaks and Fraction of Reads in Peaks called using MACS2 on samples containing the indicated number of cells. Random samples of mapped fragments were drawn, mitochondrial reads were removed and MACS2 was used to call (narrow) peaks. The number of peaks called for each sample is a measure of sensitivity, and the fraction of reads in peaks (FRiP, right) is a measure of specificity calculated for each sampling from 50,000 to 16 million fragments. Nuclei data are from a previously described experiment (Example 1, Kaya-Okur H S, Janssens D H, Henikoff J G, Ahmad K, Henikoff S. Efficient low-cost chromatin profiling with CUT&Tag. Nat Protoc. 2020; 15 (10): 3264-83).

FIGS. 2A-2F. High temperatures improve yield of small mouse fragments with FFPE-CUTAC. 2A) Scheme, where TL Prot K is Thermolabile Proteinase K (New England Biolabs). Created with BioRender.com. 2B) Arrhenius plot showing the recovery of fragments mapping to the Mm 10 build of the mouse genome as a function of temperature. Deparaffinized FFPEs were scraped into cross-link reversal buffer (Example 1, Oba U, Kohashi K, Sangatsuda Y, Oda Y, Sonoda K H, Ohga S, et al. An efficient procedure for the recovery of DNA from formalin-fixed paraffin-embedded tissue sections. Biology methods & protocols. 2022; 7 (1): bpac014) containing 0.05% Triton-X100, needle-extracted, and divided into PCR tubes for incubation in a thermocycler at the indicated temperatures. 2C) Same as (B) except for fragments mapping to the Rhodococcus erythropolis genome. 2D) Scatter plots and R2 correlations between total fragments recovered versus R. erythropolis and the summed totals for 6 other bacterial species discovered in BLASTN searches of unmapped reads (Escherichia coli, Leifsonia species, Deinococcus aestuarii, Mycobacterium syngnathidarum, Vibrio vulnificus, and Bacillus pumilus). 2E) Comparison of average overall length distributions between tumor and normal brain, combining samples from all 3 brain tumors (YAP1, PDGFB and RELA). RNAPII-Ser5p: 15 samples; RNAPII-Ser2,5p: 15 samples; H3K27ac: 15 samples; 50:50 mixture of RNAPII-Ser5 and RNAPII-Ser2,5p: 14 samples. For each sample, mouse fragment lengths were divided by the total number of fragments before averaging. Lengths are plotted at single base-pair resolution. 2F) Average length distributions for on-slide samples grouped by cancer driver transgene (YAP1: 12 samples; PDGFB: 7 samples; RELA: 12 samples) and Normal brain: 10 samples. Data are presented as mean values+/−SD in panels E-F. S

FIGS. 3A-3C. Length distributions of DNAs tagmented by CUT&Tag of FFPEs. 3A) Lengths are plotted at single basepair resolution. 3B) Same as (A) except smoothed with a 5-bp window to iron out the 10-bp periodicity and facilitate comparisons. 3C) Same as (A) except for Mm10 ChrM (mitochondrial) fragments from the same FFPEs as used for (A-B). The length distribution of Mm10 ChrM fragments from mouse 3T3 cells is plotted for reference.

FIGS. 4A-4G. Comparison of H3K27ac FFPE-CUTAC to FACT-seq and CUT&Tag of frozen unfixed samples. (4A-4D) Representative examples of housekeeping gene regions were chosen to minimize the effect of cell-type differences between FFPE-CUTAC (three brain tumors) and FACT-seq (kidney). Forebrain H3K27ac ChIP-seq and ATAC-seq samples from the ENCODE project are shown for comparison, using the same number of fragments (20 million) for each sample. Also shown are tracks from FFPE-CUTAC samples using an antibody to RNAPII-Ser2,5p. A track for Candidate cis-Regulatory Elements (cCREs) from the ENCODE project is shown above the data tracks, which are autoscaled for clarity. (4E-4F) Number of peaks and Fraction of Reads in Peaks called using MACS2 on samples containing the indicated number of cells. 4G) Cumulative log-log plots of cCRE overlap with normalized counts from each sample as indicated.

FIGS. 5A-5D. Volcano plots for pairwise comparisons between FFPE-CUTAC samples. The Degust server (degust.erc.monash.edu/) was used with Voom/Limma defaults to generate volcano plots, where replicates consisted of a mix of samples run in parallel or on different days on FFPE slides from 8 different brain samples. (3 Normal, 3 YAP1, 1 PDGFB, 1 RELA). Input for each sample was 10-25% of an FFPE slide, which ranged from ~50,000-100,000 cells per 10-micron section. 5A) Comparisons based on RNAPII-Ser5p using average normalized counts per base-pair for each cCRE, applying the Empirical Bayes Voom/Limma algorithm for pairwise comparisons using the other datasets as pseudo-replicates to increase statistical power. Replicate numbers: Normal: 13; YAP1: 14, PDGFB: 3; RELA: 2; 5B) Replicate numbers: Normal: 5; YAP1: 6; PDGFB: 3; RELA: 3. 5C) Same as (A) for H3K27ac. Replicate numbers: Normal: 10; YAP1: 12; PDGFB: 5; RELA: 7. 5D) Datasets from multiple FFPE-CUTAC experiments for each antibody (RNAPII-Ser5p, RNAPII-Ser2,5p or H3K27ac) or antibody combination (RNAPII-Ser5p+RNAPII-Ser2,5p) were merged, then down-sampled to the same number of mapped fragments for each genotype. These 16 datasets (4 antibodies×4 genotypes) were compared against each other with Voom/Limma using the other 14 datasets as pseudo-replicates . . .

FIGS. 6A-6L. Top significant differences between tumor and normal and between tumors based on RNAPII-Ser5p FFPE-CUTAC comparisons. 6A-6E) IGV tracks centered around the cCREs with the most significant difference across all pairwise comparisons (FDR=5×10⁻⁵-2×10⁻⁴). To enrich for regulatory elements within the span of each cCRE, we used the maximum value. 6F-6L) Tracks centered around the cCRE for each of the strongest signals with FDRs<0.05, ordered by increasing FDR (0.003-0.045).

FIGS. 7A-7B. Comparisons between FFPE-CUTAC and RNA-seq. 7A) Scatterplots of representative FFPE-CUTAC replicate samples from RNAPII-Ser5p, RNAPII-Ser2,5p, RNAPII-Ser5p+RNAPII-Ser2,5p and H3K27ac. 7B) Examples of the best distinguished samples based on FDR. Pairwise comparisons between samples were used to choose examples in rank order based on FFPE-CUTAC FDR.

FIGS. 8A-8D. FFPE-CUTAC distinguishes tumor from normal tissue within the same FFPE section. 8A) RELA drives well-defined epidendymomas where dissection following tagmentation and transfer of whole sections to PCR tubes after RNAPII-Ser5p FFPE-CUTAC post-tagmentation successfully separated tumor from normal tissue with volcano plot results similar to that for RELA versus Normal brains (FIGS. 5A-5D). 8B) In contrast, PDGFB-driven gliomas are relatively diffuse, and separation of sections post-tagmentation resulted in fewer significant target cCREs. 8C) Left: Volcano plot for FFPE Slide 1 (2 tumor 4 normal sections) using two other slides with 3 tumor and 5 normal sections as pseudo-replicates. Top RELA FFPE hits based on FDR<0.01 and greatest fold-change are circled). Middle: Volcano plot for a PDGFB slide (3 tumor, 4 normal). Right: Volcano plot for a normal brain (5 versus 5 replicates). Replicate tracks for the two top upregulated (Col1Al) and down-regulated (Mir124a-1 hg) loci are shown, group-autoscaled, where dark marks dots with FDR<0.05. 8D) Tracks for the RELA Tumor-versus-Normal experiments are shown for Collal (left) and Mir 124a-1 hg (right) color-coded and group-autoscaled for each replicate FFPE slide dissected.

FIGS. 9A-9I. FFPE-CUTAC produces high-quality data from liver FFPEs. 9A-9D) Representative tracks of liver tumor and normal liver FFPE-CUTAC and FACT-seq samples at the housekeeping gene regions depicted in FIGS. 4A-4D. A track for Candidate cis-Regulatory Elements (cCREs) from the ENCODE project is shown above the data tracks, which are autoscaled for clarity. 9E-9F) Number of peaks and Fraction of Reads in Peaks (FRiP) called using MACS2 on samples containing the indicated number of cells for 7 liver tumor (magenta), 6 normal liver (blue) and 2 normal liver FACT-seq (green) samples. 9G) Cumulative logio plots of normalized counts intersecting cCREs versus logio rank for representative liver samples, where red marks dots with FDR<0.05. 9H) Voom/Limma volcano plot for the 7 liver tumors versus 6 normal liver samples. 91) Control volcano plot in which three liver tumor samples and 3 normal livers were exchanged for Voom/Limma analysis.

FIGS. 10A-10C. Modified CUT&Tag-direct for whole cells and FFPEs. 10A) Scheme. 10B) Representative Tapestation profiles for whole-cell CUT&Tag-direct. A log culture of K562 cells was supplemented with 10% DMSO, concentrated to 2 million cells/ml, aliquoted, slow-frozen in Mr. Frosty containers and stored at −80° C. An aliquot was thawed and 15-60 μL was dispensed into PCR tubes for CUT&Tag-direct using an H3K27me3 antibody (CST cat. no. 9733). 10C) Tapestation profiles for FFPE CUTAC samples pre-incubated 85° C. 12 hr using four different antibodies on samples. Each sample was divided 3/4-1/4 in the TAPS-wash before fragment release. Antibodies (1:25): RNAPII-Ser5p Cell Signaling Technology #13523, RNAPII-Ser2,5 Cell Signaling Technology #13546, H3K27ac: Abcam #4729. A 10 μm section of a mouse brain tumor FFPE was deparaffinized using Option 1 (xylene). Note that both the CUTAC peaks the high-molecular weight smears scale with the amount of sample, likely representing ambient RNAs, which do not interfere with flow cell runs.

FIG. 11. Volcano plots of RNA-seq comparisons. Yap1: 3 replicates; Pdgfb: 4 replicates; RelA: 4 replicates; Naïve: 7 replicates.

FIG. 12. Exemplary home workbench for CUT&Tag. Photo of example home workbench setup used for experiments in Example 1 protocol. A typical experiment begins by mixing cells with activated ConA beads in 32 single PCR tubes, with all liquid changes performed on the magnet stands. The only tube transfer is the removal of the purified sequencing-ready libraries from the SPRI beads to fresh tubes for Tapestation analysis and DNA sequencing.

FIG. 13. Image of part of an FFPE mouse brain tumor 10 μm shard after needle dispersion and 90° C. pre-treatments, stained with Trypan blue.

FIG. 14. Left: Reducing DNA contamination increases yields. Right: Arrhenius plot illustrates how high temperatures decrease the fraction of contaminating DNA, which when denatured is not a substrate for Tn5.

FIG. 15. Image of example transfer of paraffinized curls to mineral oil.

FIG. 16. Image of beads bound to tissue shards.

FIG. 17. Tapestation profiles for FFPE CUTAC samples pre-incubated 85° C. 12 hr using four different antibodies on samples. Each sample was divided 3/4-1/4 in the TAPS-wash before fragment release. Antibodies (1:25): RNAPII-Ser5p Cell Signaling Technology #13523, RNAPII-Ser2,5 Cell Signaling Technology #13546, H3K27ac: Abcam #4729. A 10 μm section of a mouse brain tumor FFPE was deparaffinized using Option 1 (xylene). Note that both the CUTAC peaks the high-molecular weight smears scale with the amount of sample. Use a 175-500 bp range for estimating molar concentration. There is no need to remove the high molecular weight smear, which is not tagmented and does not interfere with the flow cell run.

FIG. 18. A gene-rich housekeeping gene region was chosen to minimize the effect of cell-type differences between FFPE-CUTAC (A RelA-driven and two replicates of a PDGFB-driven brain tumor) and FACT-seq and CUT&Tag (kidney data from Zhao L, Xing P, Polavarapu V K, Zhao M, Valero-Martinez B, Dang Y, et al. FACT-seq: profiling histone modifications in formalin-fixed paraffin-embedded samples with low cell numbers. Nucleic Acids Res. 2021; 49 (21): e125.). A forebrain H3K27ac ChIP-seq sample from the ENCODE project is shown for comparison, using the same number of fragments (10 million) for each sample. Also shown are tracks from FFPE-CUTAC samples using an antibody to RNAPII-Ser2,5p. A track for Candidate cis-Regulatory Elements (cCREs) from the ENCODE project is shown above the data tracks, which are autoscaled for clarity.

FIG. 19. On-slide FFPE-CUTAC. Schematic of an example protocol.

FIG. 20. Image of a small slide holder that will hold two plastic film-wrapped slides without touching or disturbing the wrap. Closing the top will allow for long incubations without drying out. For small tissue sections (e.g., 1 cm²), using small plastic wrap squares that cover the sample but do not wrap around the slide will require proportionally less volume, saving on reagent costs.

FIG. 21. Optional setup for incubating multiple slides with the same solution.

FIG. 22. Example of an incubation step. On-slide FFPE-CUTAC was performed using a rabbit RNA Polymerase II Serine-5 monoclonal antibody (Cell Signaling Systems #13523). Four slides from two mouse RELA transgene-driven ependymoma FFPE blocks (5 and 10 μm from the 33005 block and 10 μm from the 33003 block) were processed in parallel. The slides were placed on top of plastic film over a black background for good visibility of tissue, slides were abutted and aligned for each incubation as indicated. About 100 μl antibody or pAG-Tn5 solution was added dropwise to cover the tissue, and the plastic film was slowly pulled over the top edge, minimizing bubbles and wrinkles. Photograph is of the samples during the pAG-Tn5 incubation. The 10 μm 33003 FFPE section was prepared on a standard microscope slide and shows partial loss of the sections with most of the tumor (pooled for tube 4), whereas the other three sections were prepared on charged slides and show full retention of samples throughout the protocol. Numbers indicate PCR tube sample.

FIG. 23. Tapestation gel image of 1/10^{th} of each SPRI-bead purified DNA eluate from an on-slide experiment.

FIGS. 24A-24C. Analyses of the data produced in an example CUTAC-FFPE experiment shown in FIGS. 20-21. 24A) Remainder of each (barcoded) sample was pooled together with other barcoded samples and sequenced on a NextSeq 2000 PE50 flow cell and the library size was estimated based on Picard Tools Mark Duplicates (68,089,523 in total) and plotted against the total number of reads (149,314,057 in total) for each sample. Total unique fragment estimates were: 10,582,472 (5 μm square), 20,708,800 (10 μm hexagon), 16,833,815 (5 μm pentagon) and 19,964,436 (10 μm triangle). 24B) Fragment length distributions of tumor and normal sections from all slides. Mean with standard deviation error bars. 24C) Volcano plot (middle panel) produced using the Degust server with Voom/Limma option, comparing the RELA-driven tumor sections version normal sections for all four slides. The input table consisted of 343,731 rows of mouse candidate cis-regulatory elements (cCREs) from ENCODE with one column for each of the 16 samples. Tracks for the cCRE with the highest Fold-change up (Igf2) and down (Mir124a-1 hg) are shown. Both Igf2 and Mir124a-1 hg account for multiple of the highest scoring cCREs indicated by circles.

FIG. 25. Examples of moist chambers using wet paper towels in a plastic tray and staining dish. When covered slides stay wet under plastic wrap rectangles or squares (for small tissue sections and reduced volumes). Slides are placed in the rack for incubation, and afterwards are placed face up on the wet paper towel in the plastic tray to wash the bottom before removing the plastic wrap and rinsing the top.

FIG. 26. A curl (white) in a 1.5 mL Eppendorf tube.

FIGS. 27A-27C. Analyses of the data produced in the experiment shown in FIGS. 20 and 21. 27A) Remainder of each (barcoded) sample was pooled together with other barcoded samples and sequenced on a NextSeq 2000 PE50 flow cell and the library size was estimated based on Picard Tools Mark Duplicates (68,089,523 in total) and plotted against the total number of reads (149,314,057 in total) for each sample. Total unique fragment estimates were: 10,582,472 (5 μm square), 20,708,800 (10 μm hexagon), 16,833,815 (5 μm pentagon) and 19,964,436 (10 μm triangle). 27B) Fragment length distributions of tumor and normal sections from all slides. Mean with standard deviation error bars. 27C) Volcano plot (middle panel) produced using the Degust server with Voom/Limma option, comparing the RELA-driven tumor sections version normal sections for all four slides. The input table consisted of 343,731 rows of mouse candidate cis-regulatory elements (cCREs) from ENCODE (cCRE combined ENCODE available at genome.ucsc.edu) with one column for each of the 16 samples. Tracks for the cCRE with the highest Fold-change up (Igf2) and down (Mir124a-1 hg) are shown. Both Igf2 and Mir124a-1 hg account for multiple of the highest scoring cCREs indicated by circles.

FIGS. 28A-28F. RNAPII-Ser5p FFPE-CUTAC directly maps hypertranscription. 28A) Model for hypertranscription in cancer: Paused RNAPII at active gene regulatory elements, such as promoters and enhancers, increases on average over the cell cycle resulting in a net proportional gain in RNAPII occupancy across the genome. Using RNAPII FFPE-CUTAC hypertranscription genome-wide can be mapped using three complementary approaches: 1) Genome-scaled Tumor (T) minus Normal (N) counts at cCREs, 2) T-N at replication-coupled histone genes and 3) Sparse Enrichment Analysis for CUT&RUN (SEACR) Tumor peak calls using Normal as the background control. 28B-28E) Bland-Altman plots showing hypertranscription mapped over the 343,731 annotated mouse cCREs for tumor and normal sections dissected posttagmentation from a 10 micron FFPE slice from each of four different paraffin blocks. Hypertranscription of a cCRE is defined as the excess of RNAPII-Ser5p in the indicated tumor over normal (Tumor minus Normal in normalized count units for Mm 10-mapped fragments pooled from the same slide). 28F)

Hypertranscription at replication-coupled histone genes. Slides used for PDGFB-2a-c were from the same paraffin block but used in different experiments, and all others were from different paraffin blocks. Numbers at right were obtained by subtracting the sum of normalized counts in the normal sections from that in the tumor sections over all 64 annotated single-exon replication-coupled histone genes, where the Standard Deviation is shown. Paired t-test: * p<0.001; ** p<0.00001.

FIGS. 29A-29I. Hypertranscription in human Tumor-vs-Normal tissues. 29A-29H) All fragments were pooled from four slides from the same paraffin block and the number of fragments equalized between tumor and normal for each of the seven cancers. Bland-Altman plots showing hypertranscription mapped over the 984,834 annotated mouse cCREs for tumor and matched normal sections from 5 micron FFPE slices. Max Diffs displays the Tumor minus Normal maximum of the seven samples for each cCRE. 291) The minor human histone gene cluster on Chr 1 is shown, where tracks are autoscaled for each Tumor (red) and Normal (blue) pair. As individual samples are not intended to represent tumor types, sample names are abbreviations (FIG. 37).

FIGS. 30A-30D. FFPE-CUTAC mitochondrial DNA signal is reduced in tumors. 30A) The percentage of normalized counts mapping to Chromosome M (ChrM=mitochondrial DNA) was calculated for FFPE-CUTAC data from four mouse brain tumor paraffin blocks driven by PDGFB, YAPI and RELA transgenes. An RNAPII-Ser5p antibody was used for the first four comparisons, and an RNAPIISer2p and histone H3K27ac antibodies were used respectively for the fifth and sixth comparisons. 30B) Same as (A) for RNAPII-Ser5p FFPE-CUTAC data for the seven human Tumor/Normal pairs used in this study. 30C-30D) ATAC-seq count data from The Cancer Genome Atlas (TCGA) (tumor) and ENCODE (normal) shows variability in ChrM percentages between tumors, consistent with our finding based on FFPE-CUTAC.

FIGS. 31A-31F. Top-ranked human cCREs based on hypertranscription correspond to SEACR Tumor-vs-Normal RNAPII-Ser5p peaks. For each of the indicated tumors (31A-31F), tracks are shown for 50-kb regions around the #1-ranked cCRE based on Tumor (dark gray) and Normal (gray) counts. Raw data tracks were group-autoscaled together for tumor (dark gray) and normal (gray), where SEACR Tumor peak calls (light gray) use Normal as the negative control. Gene annotations and cCREs (black rectangles) are shown at top.

FIGS. 32A-32F. Hypertranscription differs between human liver tumors. 32A-32D) Top-ranked cCREs based on liver tumors 1 and 2 (dark gray) and matched normal (gray) counts. Tumor/Normal tracks and Tumors 3-5 are group-autoscaled. 32E) Same as (A), except for the minor histone gene cluster on Chromosome 1. 32F) Levels of hypertranscription differ between different hepatocarcinomas (Tumor 1: solid lines, Tumor 2 dotted lines, where tumor is dark gray and normal is gray). For each tumor and normal sample, we counted the number of mapped fragments spanning each base-pair in a cCRE scaled to the human genome and averaged the number of counts over that cCRE. Rank-ordered based on tumor minus normal representing global upregulation, and conversely rank-ordered cCREs based on normal minus tumor representing global downregulation. With such a large collection of loci, the a priori expectation is that the rank-ordered distribution of differences between tumor and normal will be approximately the same regardless of whether the differences are based on tumor minus normal or normal minus tumor. For clarity, rank-ordered differences were plotted on a log 10 scale.

FIG. 33. Tight clustering of tumor samples. UMAP of 114 human tumor samples (upper panel). Lower panel, Same as upper panel except shaded for sequencing depth and indicating homogeneous tumor clusters.

FIGS. 34A-34F. Hypertranscription identifies likely HER2 amplifications and regions of linkage disequilibrium. 34A) Raw data tracks for the 1-Mb region on Chromosome 17q21 were group-autoscaled together for tumor and normal, where SEACR Tumor peak calls use Normal as the negative control. Broad regions of prominent hypertranscription, indicate likely HER2 amplifications in both tumors. 34B) Raw data tracks for the 250-kb 17q12 region amplified in Br but evidently not in Co. 34C) Raw data tracks for the CCNK promoter region, where the normalized count increase in the Br tumor relative to normal over the 10-kb region shown is 5.4-fold and for Co is 2.1-fold and the range for the other five tumors is 0.9-2.5. 34D-34E) The two 1-Mb regions displayed in (C-D) were tiled with 1-kb bins and count density curves were fitted for all 7 tumor-normal pairs. Arrows mark the locations of indicated promoter peaks in the breast and colon tumors. 34F) Individual broad summits in (D-E) were zoomed-in and rescaled on x-axis centered over the indicated promoter peak and superimposed over raw normalized count tracks scaled to the height of the central peak.

FIGS. 35A-35H. RNAPII-Ser5p FFPE-CUTAC shows stronger and more frequent changes in up-regulation than down-regulation of cCREs. Related to FIG. 28. The Voom/Limma option of the Degust server (degust.erc.monash.edu/) was applied to mouse cCRE RNAPII-Ser5p FFPE-CUTAC data from pooled replicates from 5 RELA and 4 PDGFB experiments. MA plots display x=log 10 (Tumor*Normal)/2 versus y=log 2 (Tumor/Normal) for normalized counts from the tumor and normal samples being compared, and red color indicates FDR<0.05. Normalized counts are the fraction of counts at each base pair scaled by the size of the Mm 10 reference sequence (2,818,974,548), so that if the counts are uniformly distributed across the reference sequence there would be one at each position. (35A-35B) Both RELA and PDGFB tumor sections show higher counts than normal sections but significant RELA changes both up and down are far stronger than PDGFB changes, confirmed in a head-to-head comparison between tumors and normal sections. (35C-35E) Same as (A-B) except using either RNAPII-Ser5p or histone H3K27ac antibodies for FFPE-CUTAC and using entire 10 μm curls divided into 4-8 samples per curl for PCR and sequencing. For MA plots, data were merged from multiple experiments and equalized by downsampling to 10 million fragments, with 4 merged replicates per sample. DAP-stained slides for each paraffin block used, with the total fraction of tumor indicated in parentheses. (35F-35H) Voom/Limma was used to construct MA plots based on individual 10 μm sections from single slides corresponding to the boxed sections on slides DAP-stained for tumor-driver transgene expression. Numbers in parentheses are percentages of tumor cells based on numbers of stained and unstained cells within the boxed sections.

FIG. 36. Hypertranscription mapped over the 343,731 ENCODE-annotated mouse cCREs categorized by regulatory element type. Related to FIG. 28. For each tumor and normal sample, we counted the number of mapped fragments spanning each base-pair in a cCRE scaled to the mouse genome and averaged the number of counts over that cCRE. We then divided up the 343,731 cCREs into the five ENCODE-annotated categories: Promoters (24,114), H3K4me3-marked cCREs (10,538), Proximal Enhancers (108,474), Distal Enhancers (211,185) and CTCF cCREs (24,072) and rank-ordered based on tumor minus normal representing global upregulation, and conversely rank-ordered cCREs based on normal minus tumor representing global downregulation. With such a large collection of loci, our a priori expectation is that the rank-ordered distribution of differences between tumor and normal will be approximately the same regardless of whether the differences are based on tumor minus normal or normal minus tumor. For clarity, rank-ordered differences were plotted on a log 10 scale. Strong hypertranscription for RELA and PDGFB-1, weak hypertranscription for PDGFB-2 and little or no hypotranscription for YAPI is seen for all classes, consistent with the Bland-Altman plots shown in FIG. 28b-28e.

FIG. 37. Photographs of 5 μm FFPE sections from human tumor and adjacent normal tissues. Related to FIG. 29. Pathology classification, age and sex were provided by the vendor (BioChain). Each image spans the width of a standard charged microscope slide, where the tissue is visible under the paraffin skin. On-slide RNAPII-Ser5p FFPE-CUTAC was applied to slides in parallel, using a total of four slides each for 100 separate samples in all to produce the data analyzed in this study. To avoid the impression that these individual tumors are representative of their tumor types, their designations are abbreviated: Br, Co, Ki, Li, Lu, Re and St.

FIGS. 38A-38X. Hypertranscription in human Tumor-vs-Normal tissues. Related to FIG. 29. 38A-38H) Same data as in FIG. 29A-29H, except plotted as in FIG. 37 to facilitate comparisons. 381-38P) Combined data from a single slide with duplicate removal. 38Q-38X) Combined data from 4 slides after removing duplicates and equalizing the number of fragments between tumor and normal sections. Number of unique fragments per sample in each Tumor/Normal pair: Br: 1,125,608; Co: 3,712,097; Ki: 2,031,893; Li: 2,983,411; Lu: 1,123,638; Re: 3,284,736; St: 719,598.

FIGS. 39A-39L. Focal hypertranscribed regulatory elements embedded in broad regions of hypertranscription on Chromosome 17q12-22. Related to FIG. 34. 39A-39F) The six most highly transcribed cCREs within the ~5 Mb region of Chromosome 17q1.2-2.2 are displayed with each tumor (dark gray) and normal (gray) pair scaled to one another so that peaks can be observed in all samples. SEACR peaks (light gray) are group-autoscaled in all panels. 39G-39L) Same as (A-F) except that all tumor-normal samples are group-autoscaled to the height of the tallest peak, where the disappearance of all the peaks except for those in Br and for MSL1 and ERBB2 in Co is evidence that peaks in these regions are strongly hypertranscribed in Br and partially in Co but not in any of the other tumors.

FIGS. 40A-40E. Weak RNAPII upregulation of RNAPII of the top-ranked loci outside of the HER2 amplicon. Related to FIG. 34. See FIG. 34C-34D for details regarding top-ranked loci outside of the HER2 amplicon.

### DETAILED DESCRIPTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art. In addition, any references cited herein are incorporated by reference in their entireties.

Unless otherwise defined, all technical and scientific terms used herein have the same meaning as commonly understood by one of skill in the art to which this invention belongs. The terminology used in the description of the invention herein is for the purpose of describing particular embodiments only and is not intended to be limiting of the invention. All publications, patent applications, patents, patent publications and other references cited herein are incorporated by reference in their entireties for the teachings relevant to the sentence and/or paragraph in which the reference is presented.

Amino acids are represented herein in the manner recommended by the IUPAC-IUB Biochemical Nomenclature Commission, or (for amino acids) by either the one-letter code, or the three-letter code, both in accordance with 37 C.F.R. § 1.822 and established usage.

Except as otherwise indicated, standard methods known to those skilled in the art may be used for cloning genes, amplifying and detecting nucleic acids, and the like. Such techniques are known to those skilled in the art. See, e.g., Sambrook et al., Molecular Cloning: A Laboratory Manual 4th Ed. (Cold Spring Harbor, NY, 2012); Ausubel et al. Current Protocols in Molecular Biology (Green Publishing Associates, Inc. and John Wiley & Sons, Inc., New York).

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate, if the specification states that a complex comprises components A, B and C, it is specifically intended that any of A, B or C, or a combination thereof, can be omitted and disclaimed singularly or in any combination.

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of +10%, +5%, +1%, +0.5%, or even±0.1% of the specified amount.

As used herein, the transitional phrase “consisting essentially of” (and grammatical variants) is to be interpreted as encompassing the recited materials or steps and those that do not materially affect the basic and novel characteristic(s) of the claimed invention. Thus, the term “consisting essentially of” as used herein should not be interpreted as equivalent to “comprising.”

The term “consists essentially of” (and grammatical variants), as applied to a polypeptide or polynucleotide sequence of this invention, means a polypeptide or polynucleotide that consists of both the recited sequence (e.g., SEQ ID NO) and a total of ten or less (e.g., 1, 2, 3, 4, 5, 6, 7, 8, 9, or 10) additional amino acids on the N-terminal and/or C-terminal ends of the recited sequence or additional nucleotides on the 5′ and/or 3′ ends of the recited sequence such that the function of the polypeptide or polynucleotide is not materially altered. The total of ten or less additional amino acids or nucleotides includes the total number of additional amino acids or nucleotides on both ends added together. The term “materially altered,” as applied to polypeptides of the invention, refers to an increase or decrease in biological activities/properties (e.g., remodeling activity) of at least about 50% or more as compared to the activity of a polypeptide consisting of the recited sequence.

As used herein, the term “polypeptide” encompasses both peptides and proteins, unless indicated otherwise.

The terms “polynucleotide,” “nucleic acid,” “nucleic acid molecule,” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, genomic DNA, chimeras of RNA and DNA, isolated DNA of any sequence, isolated RNA of any sequence, synthetic DNA of any sequence (e.g., chemically synthesized), synthetic RNA of any sequence (e.g., chemically synthesized), nucleic acid probes and primers. A polynucleotide can comprise modified nucleotides, such as methylated nucleotides and nucleotide analogs or derivatives (e.g., inosine or phosphorothioate nucleotides). Such nucleotides can be used, for example, to prepare nucleic acid molecules that have altered base-pairing abilities or increased resistance to nucleases.

The term “modulate,” “modulates,” or “modulation” refers to enhancement (e.g., an increase) or inhibition (e.g., a decrease) in the specified level or activity.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold and/or can be expressed in the enhancement and/or increase of a specified level and/or activity of at least about 1%, 5%, 10%, 15%, 25%, 35%, 40%, 50%, 60%, 75%, 80%, 90%, 95% or more.

The term “inhibit” or “reduce” or grammatical variations thereof as used herein refers to a decrease or diminishment in the specified level or activity of at least about 1, 5, 10, 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

The term “contact” or grammatical variations thereof refers to bringing two or more substances in sufficiently close proximity to each other for one to exert a biological effect on the other.

The term DNA Integrity Number (DIN) or RNA Integrity Number (RIN) refers to a numerical value quote as a measure of the quality of the DNA or RNA. A DIN/RIN can be measured using DNA/RNA quantification machines, for example, by the Agilent Tapestation® or Bioanalyzer. A DIN/RIN value ranging between 1 and 10 can be assigned to the DNA/RNA, with 10 being completely intact material and 1 being completely degraded. In some embodiments, the DIN score for an FFPE sample is evaluated subsequent to deparaffinazation of the sample, and may comprise extracting cells, nuclei, or DNA isolated from the sample. In some embodiments, the high sensitivity of the present methods allows evaluation of samples with a DIN score of at least 5, 4.5, 4, 3.5, 3, 2.5, or 2.

Previous CUT&Tag-based methods show limited compatibility with analysis of FFPE samples. In some embodiments, the present invention relates to the use (and improvements to) CUTAC to enable high-throughput FFPE tissue analysis. CUTAC methods are described, for example, in International Patent Publication WO 2022/056309, incorporated herein by reference in its entirety. Applicants herein leverage the high sensitivity of CUTAC along with further revisions to those methods to get high signal to noise in FFPE samples, including highly degraded FFPE samples. Indeed, the CUTAC workflow produces <120-bp fragments that not only increases mapping resolution and sensitivity, but also helps overcome DNA degradation caused by fixation and cross-linking by increasing the likelihood of two successful tagmentation events occurring on an intact segment of DNA. In some embodiments, the formaldehyde treatment in a FFPE sample forms covalent bonds between DNA and lysine-rich histones in nucleosomes rendering them inflexible, so that open chromatin gaps are the accessible DNA in the nucleus. As detailed herein, by using antibodies, for example, to the phosphorylated RNAPII heptapeptide repeat present in 52 lysine-free tandem copies or to the abundant histone H3K27ac mark of active regulatory elements, the presently disclosed methods take advantage of the hyperaccessibility and abundance of the targeted epitope and the impermeability of lysine-rich histone cross-linked chromatin to achieve exceptional signal-to-noise from FFPE samples.

In some embodiments, the disclosed methods can use RNAPII to map the transcriptional machinery itself directly on the DNA regulatory elements, such that direct measurements of transcription initiation are obtained that can characterize hypertranscription at active regulatory elements genome-wide, rather than inferences based on estimating steady-state mRNA abundances. Accordingly, in some embodiments, the present invention is related to methods for measuring hypertranscription by quantifying incremental increases or decreases in RNAPII over hundreds of thousands of loci, allowing high resolution results while using low sequencing depth without reference to external information and allowing detection of genome-wide hypertranscription. The methods of measuring hypertranscription disclosed herein allow identification of loci amplifications and probable clonal selection events without relying on reference to any external data. The simple workflow of FFPE-CUTAC methods described herein can be utilized with automation to allow for routine cancer screening and other personalized medicine applications. Advantageously, the methods can be performed rapidly at low-cost (~$50 per sample) providing value as a general clinical diagnostic and research tool.

In some embodiments, an in situ method of mapping the location of a protein on chromatin in a cell from a FFPE sample is provided, comprising the steps of treating the FFPE sample to remove the paraffin; permeabilizing the sample; contacting the sample with a first affinity reagent that specifically binds to a chromatin protein, wherein the first affinity reagent is coupled to at least one transposome comprising: at least one transposase; and a transposon comprising: a first DNA molecule comprising a first transposase recognition site; and a second DNA molecule comprising a second transposase recognition site; activating the at least one transposase under low ionic conditions, thereby cleaving and tagging chromatin DNA with the first and second DNA molecules; excising the tagged DNA segment associated with the chromatin protein; and determining the nucleotide sequence of the excised tagged DNA segment, thereby mapping the genomic location of the targeted protein on chromatin.

In some embodiments, a DNA-based in situ method for measuring transcription in a cell from a FFPE sample is provided, comprising: treating the FFPE sample to remove the paraffin; permeabilizing the sample; contacting the sample with a first affinity reagent that specifically binds to a protein involved in transcription regulation, wherein the first affinity reagent is coupled to at least one transposome comprising: at least one transposase; and a transposon comprising: a first DNA molecule comprising a first transposase recognition site; and a second DNA molecule comprising a second transposase recognition site; activating the at least one transposase under low ionic conditions, thereby cleaving and tagging chromatin DNA with the first and second DNA molecules; excising the tagged DNA segment associated with the protein involved in transcription regulation; and determining the nucleotide sequence of the excised tagged DNA segment, thereby mapping transcriptional activity on chromatin.

In some embodiments, treating the FFPE sample to remove the paraffin, also referred to herein as deparaffinizing, can comprise applying high heat to the sample. As used herein, high heat can include heating a sample above 50° C., which may be at least 50° C., 55° C., 60° C., 65° C., 70° C., 75° C., 80° C., 85° C., or 90° C. In some embodiments removing paraffin comprises incubating the sample at least at 75° C., 80° C., 85° C., or 90° C. The sample may be heated for between about 5 minutes and 3 or more hours (See, e.g., Example 3, step 25 and accompanying note describing extension of incubation times), which may be dependent at least in part on the sample type, for example, whether sample is tissue on a slide, or cells or nuclei associate with beads, or samples in nanowells. In some embodiments, removing paraffin comprises heating at 85-90° C. for between 1 hour and 16 hours. In some embodiments, the method comprises isolating nuclei with heat and minimal mechanical processing. In some embodiments, the method comprises isolating nuclei without (i.e., is devoid of) enzymatic processing of the tissue for isolating nuclei.

In some embodiments, the sample is further treated with cross-link reversal buffer, which may comprise Tris(hydroxymethyl)aminomethane hydrochloride (Tris HCl) and/or Ethylenediaminetetraacetic acid (EDTA). In some embodiments, a cell (or nucleus) in the sample is permeabilized with a detergent, e.g., by digitonin. In some embodiments, a cell and/or nucleus of the cell in the sample is permeabilized by the step of removing the paraffin with heat in the cross-link reversal buffer. In some embodiments, the addition of Triton® X100 to buffer solutions used in several steps of the methods helps maintain cell permeability. In some embodiments, crowding reagents are included in buffer solutions to increase tagmentation efficiency and/or signal-to-noise. In some embodiments, the method comprises separating the sample into tissue fragments, cells, or nuclei before or after the step of permeabilizing the sample. The sample may comprise a tissue sample, e.g., a curl, a slice, a punch, or other FFPE tissue sample. The sample can comprise about 1,000 cells to about 2,000,000 cells or more, or any range therein. In some embodiments, the sample is separated into single cells and/or nuclei prior to contacting the sample with the first affinity reagent. In some embodiments, the method is performed on fragments of a sample that has been mechanically digested. An example embodiment of mechanical separation is provided in Example 3 describing the mortar and pestle protocol. In some embodiments, the sample is sheared. In some embodiments, methods can comprise obtaining tissue from a slide, for example, by optionally dicing or otherwise sectioning the tissue sample on the slide and scraping the tissue from the slide and further forcing a solution comprising the tissue sample multiple times through a needle (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30 or more times) to thereby provide fragments of a sample.

In an example embodiment, the method can be performed on a FFPE curl. In one embodiment, the step of treating the FFPE curl sample comprises adding mineral oil to the sample and heating the sampled at 85-90° C. for between 3-10 minutes (e.g., 5 minutes) to melt paraffin, and homogenizing the sample with a pestle. The method can comprise adding cross-link reversal buffer comprising Tris-HCl at a pH between about 7.5 and 9.0 (e.g., pH8.0) and amine functionalized paramagnetic beads in a ratio of, for example 1:10. In some embodiments, homogenization can be repeated followed by a subsequent addition of cross-link reversal buffer. In some embodiments, the cross-link buffer utilized is warmed prior to addition. In some embodiments, the samples are incubated at 85-90° C. for between 1 hour-14 hours followed by vortexing, centrifuging, and removing the mineral oil. Mineral oil can then be added, mixed by inversion, centrifuged and subsequently removed with the exception of a thin oil layer at the top of the sample. The method can further comprise adding paramagnetic beads, for example, agarose glutathione paramagnetic beads, and mixing the samples. The method can comprise exposing the samples comprising the paramagnetic beads to a strong magnet, followed by removing the supernatant, and re-suspending the remaining bead-bound homogenate in a buffer comprising Triton® X-100 and optionally HEPES pH 7.5, NaCl, spermidine, EDTA, and/or EDTA-free protease inhibitor prior to adding a first affinity reagent. In some embodiments, less than 10% of a curl, for example, 5% of a curl, is sufficient for generating a single library using the methods described herein.

In some embodiments, the method is performed on a solid support, for example a bead, a slide, a well (e.g., a microwell or nanowell) and/or the wall of a microtiter plate. The bead may be an amine-functionalized bead, for example, an agarose-glutathione bead or a lectin-coated bead (e.g., Concanavalin A). In some embodiments, the bead is a magnetic bead.

In some embodiments, the method is performed directly on a slide comprising the sample, e.g., a tissue sample. In some embodiments, the method performed on a slide produces spatially resolved results, as described further herein. In some embodiments, the method further comprises tagging each of a plurality of cells with a cell specific barcode or combination of barcodes unique to a location in a three-dimensional plurality of cells. Labeling can comprise inserting barcodes via transposase transposition or other ligation techniques (e.g., splint ligation) that can be followed by high-throughput sequencing to thereby allow spatial-resolution genome-wide mapping of chromatin protein or a protein involved in transcription regulation in tissue at a cellular level. The method can further comprise the step of imaging the three-dimensional plurality of cells prior to the step of excising the tagged DNA. IHC/IF imaging could be used to register histology information to spatial sequencing data. In some embodiments, integrating cell morphology information with spatial epigenomic mapping may provide deeper insights into how tissues change due to aging, injury, disease and/or treatment. In some embodiments, cells comprising tags, e.g., DNA barcodes, for example fluorescently labelled DNA oligomers, are imaged to thereby correlate the cell and its corresponding DNA barcodes to allow for identification of tracking of the cell location.

In some embodiments, the methods comprise using contaminating bacterial DNA as a calibration standard to normalize samples. In some embodiments, the contaminating bacterial DNA is Rhodococcus DNA. As described in the working examples, FFPE samples may be contaminated with the gram-positive bacterium Rhodococcus erythropolis and utilizing Rhodococcus DNA as the calibrating may avoid challenges when using spike-in controls with a FFPE sample. In some embodiments, the method can comprise using Rhodococcus DNA and/or nucleosome-based spike-ins. In some embodiments, methods comprise using nucleosome-based spike-ins (e.g. containing histone PTMs or other epitopes in chromatin associated protein) as previously described in, for example, International Patent Publication Nos. WO 2015117145, WO 2013184930, WO 2020132388, and WO 2020168151.

Methods may further comprise the step of deproteinating the DNA segment with an enzyme, e.g., a proteinase. In some embodiments, the method comprises treating the sample with a serine protease, e.g., proteinase K, prior to excising the tagged DNA segment. The proteinase K can be provided in a solution comprising SDS. The SDS may be used at greater than 0.5%, for example, greater than 0.6%, 0.7%, 0.8%, 0.9%, 1.0%, 1.1%, 1.2%, 1.3%, 1.4%, or 1.5%. In some embodiments, the method comprises contacting the tagged chromatin DNA segments with an SDS solution comprising proteinase K, for example 5%, 4%, 3%, 2%, 1%, 0.75%, or 0.5% SDS. In some embodiments, excising the tagged DNA can be performed using heat, for example, at a temperature of at least 35° C., 40° C., 45° C., or 50° C. In some embodiments, the SDS is supplemented with a 1:10 proteinase K to a solution used for fragment release. In some embodiments, the tagged DNA segments are contacted at 30° C. to 45° C. (e.g., 37° C.) for between 0.5 hours-2.5 hours (e.g., 1 hour), followed by 50° C. to 65° C. (e.g., 58° C.) for 0.5 hours-2.5 hours (e.g., 1 hour). In some embodiments, the step can be quenched by adding a solution of Triton®-X100, for example, 1% to 12% Triton®-X100 (e.g., 6%). The proteinase K can be a thermolabile proteinase K, which was cloned from Engyodontium album (formerly Tritirachium album) and mutagenized to increase thermolability of the enzyme available from New England Biolabs. In one aspect, a supernatant containing the cleaved segments is optionally treated with a proteinase, and DNA is quantified, for example, with imaging of stained DNA of the cleaved segments.

In some embodiments, the sample is contacted with a first affinity reagent that specifically binds to a chromatin protein or a protein involved in transcription regulation. In some embodiments, a protein involved in transcription regulation can include proteins that localize near accessible (or “open) chromatin (e.g., H3K4me2 or RNAPIIS5p). For example, an affinity reagent to a phosphoform of the C-terminal domain of RNAPII can be used in the methods described herein. The initiation form of RNAPII, which has a serine-5 phosphate on the repeated heptameric C terminal domain of the largest subunit (referred to as RNAPIIS5P), precisely aligns with transcription-coupled chromatin accessibility. The elongation form of RNAPII, which has a serine-2 phosphate on the repeated heptameric C-terminal domain of the largest subunit (referred to as RNAPIIS2P), also precisely aligns with transcription-coupled chromatin accessibility. Example phosphoforms of the C-terminal domain of RNAPII, include RNAPII-Ser2, RNAPII-Ser5, RNAPII-Ser7, RNAPII-Ser2/5, or RNAPII-Ser5/7 and an affinity reagent such as an antibody that specifically binds RNAPII can be utilized for assays related to transcription regulation and/or chromatin accessibility. Affinity reagents for chromatin proteins include, but are not limited to, for example, reagents that specifically bind to markers for negative regulatory elements (e.g., H3K27me3 or H3K9me3).

In some embodiments, the sample is contacted with a first affinity reagent that specifically binds to a targeted chromatin protein or a protein involved in transcription regulation. In some embodiments, the formaldehyde treatment of a FFPE sample forms covalent bonds between DNA and lysine-rich histones in nucleosomes rendering them inflexible, so that open chromatin gaps are the accessible DNA in the nucleus. In some embodiments, by using antibodies to the phosphorylated RNAPII heptapeptide repeat present in 52 lysine-free tandem copies or to the abundant histone H3K27ac mark of active regulatory elements, the presently disclosed methods can take advantage of the hyperaccessibility and abundance of the targeted epitope and the impermeability of histone cross-linked chromatin to achieve exceptional signal-to-noise.

In some embodiments, the first affinity reagent is directly coupled to at least one transposase. In some embodiments, the at least one transposase comprises a Tn5 transposase.

In some embodiments, the first affinity reagent and transposase are disposed in a fusion protein. In some embodiments, the first affinity reagent is indirectly coupled to the at least one transposase. In some embodiments, the transposase is linked to a specific binding agent that specifically binds the first affinity reagent.

In some embodiments, the first affinity reagent is bound by a second affinity reagent. In some embodiments, the method further comprises contacting the cell with a second affinity reagent that specifically binds the first affinity reagent, and wherein the transposase is linked to a specific binding agent that specifically binds the second affinity reagent. In some embodiments, the method further comprises contacting the cell with a second affinity reagent that specifically binds the first affinity reagent, contacting the cell with a third affinity reagent that specifically binds the second affinity reagent, and wherein the transposase is linked to a specific binding agent that specifically binds the third affinity reagent. In some embodiments, a second affinity reagent is bound by a third affinity reagent.

In some embodiments, the first, second, or third affinity reagent is directly coupled to the at least one transposome. In some embodiments, the first, second, or third affinity reagent is indirectly coupled to the at least one transposome. In some embodiments, the transposome comprises a fusion protein of the transposase and the binding agent. For example, the transposome can comprise a Tn5 transposase domain and protein A or a binding domain thereof, protein G or a binding domain thereof, or a protein A/G hybrid binding domain.

In some embodiments, the first, second, and/or third affinity reagents independently is or comprises an antibody, an antibody-like molecule, a DARPin, an aptamer, a chromatin-binding protein, other specific binding molecule, or a functional antigen-binding domain thereof. In some embodiments, the antibody-like molecule is an antibody fragment and/or antibody derivative. In some embodiments, the antibody-like molecule is a single chain antibody, a bispecific antibody, an Fab fragment, an F(ab)2 fragment, a VHH fragment, a VNAR fragment, or a nanobody. In some embodiments, the single-chain antibody is a single chain variable fragment (scFv), or a single-chain Fab fragment (scFab). In some embodiments, the first, second, and/or third affinity reagent is an antibody to a phosphoform of the C-terminal domain of RNA polymerase II (RNAPII), such as RNAPII-Ser2, RNAPII-Ser5, RNAPII-Ser7, RNAPII-Ser2/5, or RNAPII-Ser5/7.

Methods can comprise activating at least one transposase under low ionic conditions. In some embodiments, the use of low-salt tagmentation after stringent washes allows for tight binding of the Tn5 transposome and allows for epitopes flanking promoters and enhancers, such as RNAPII epitopes, to release subnucleosomal fragments preferentially, where tagmentation occurs within gaps in the chromatin landscape where these epitopes are located. As used herein, low ionic conditions comprise an ionic concentration of less than 10 mM. In some embodiments, activating the at least one transposase under low ionic conditions can comprise contacting the transposase with a sufficient amount of Mg⁺⁺ (such as in the salt form of MgCl2 or MgSO₄), for example, from about 0.1 mM Mg⁺⁺ to about 10 mM Mg⁺⁺. In some embodiments, the low ionic conditions comprise a solution of MgCl₂ and/or TAPS buffer, for example, MgCl₂ at 10 mM or less and/or TAPS buffer at 5 mM or less.

In some embodiments, activating the at least one transposase under low ionic conditions is characterized by low monovalent ionic concentration of less than about 10 mM, for example, between about 1 mM to about 10 mM, about 2 mM to about 9 mM, about 3 mM to about 8 mM, about 4 mM to about 7 mM, about 5 mM to about 6 mM, or any range therein. In some embodiments, the salt component of the reaction environment is NaCl, but other sources of monovalent ions are possible. The monovalent ions can be supplied by salts with monovalent cations such as Na+, Lit, etc., or anions such as Cl-. In some embodiments, the low ionic conditions can further comprise 1,6-hexanediol, a strongly polar aliphatic alcohol, and/or 10% dimethylformamide, a strongly polar amide. See, e.g., Steven Henikoff, Jorja G Henikoff, Hatice S Kaya-Okur, Kami Ahmad, Efficient chromatin accessibility mapping in situ by nucleosome-tethered tagmentation, eLife 9: e63274 (2020). In some embodiments, the step of contacting the permeabilized cell with the first affinity reagent and/or the step of activating the at least one transposase and tagging the chromatin DNA are performed with a buffer comprising Triton® X-100 (octyl phenol ethoxylate). In some embodiments, Triton® X-100 is provided in a buffer at 20% by weight or less, for example, 20%, 15%, 10%, 9%, 8%, 7%, 6%, 5%, 4%, 3%, 2%, 1%, 0.5%,, 0.1%, 0.09%, 0.08%, 0.07%, 0.06%, 0.05%, 0.04%, 0.03% or 0.02% or less. In some embodiments, the buffer comprising Triton® X-100 is provided in a ratio in solutions (e.g., transposases solution, primary or secondary affinity reagent (antibody) solution) at 1:15-1:30, for example 1:20 or 1:25, solution: buffer comprising Triton® X-100. In some embodiments, one or more of the steps of contacting the sample with a first affinity reagent (e.g., antibody) which may comprise incubation, binding of a transposome, and activating at least one transposase to thereby cleave and tag DNA (e.g., tagmentation) utilize buffers comprising Triton®-X100, for example, 0.05% Triton®-X100.

In some embodiments, the inclusion of Triton®-X100 is provided in buffers, which, without being bound by any particular theory, maintains cell permeability without disrupting nuclei and/or improves bead behavior. In some embodiments, where tagged DNA segments are released utilizing proteinase K, a solution comprising Triton®-X100 can be used to quench the reaction, i.e., sequesters SDS in micelles. In some embodiments, the method can be performed on whole cells without the need to purify nuclei.

In some embodiments, the tagged DNA segment that is excised from chromatin is isolated by capturing supernatant in which the tagged DNA segment is released. In certain embodiments, the excised chromatin DNA fragments are purified by immobilizing the fragments on a solid support, such as a bead, membrane, or surface (e.g,. a well or tube) that is coated with an affinity molecule suitable for immobilizing the excised chromatin DNA. In certain embodiments, the affinity molecule is silica or magnetic beads (SPRI beads). In certain embodiments, a library (e.g., for next generation sequencing applications, such as Illumina® sequencing (Illumina® Inc., San Diego, CA)) is constructed on magnetic particles. The same DNA absorbing magnetic beads can then be used to purify the resulting library. In certain embodiments, the excised chromatin DNA are purified after they have been released from the specific chromatin-associated factor and or antibody with which or to which the nucleic acid fragments were bound. In some embodiments, the methods yield <120-bp fragments (e.g., 115 bp, 110 bp, 100 bp, 95 bp, 90 bp or less) released which is relatively robust to the serious DNA degradation that occurs during cross-link reversal. In some embodiments, the method comprises performing PCR. In some embodiments, PCR is performed with an extension step, for example, 10 sec 98° C. denaturation, 30 sec 63° C. annealing and 1 min 72° C. extension for 10-14 cycles, for example, 12 or 13 cycles.

The isolated tagged DNA segment that is excised from the chromatin can be subject to further analysis, such as size characterization, or full sequencing. In some embodiments, a further advantage of providing an affinity surface in a well or as a bead, e.g., magnetic beads, is that the disclosed methods may be adapted for parallel processing of multiple samples, such as in a 96-well format or microfluidic platform, from starting chromatin material to the end of a sequencing library construction and purification. In some embodiments, the methods herein employing CUTAC can be used in conjunction with spatial analysis. For example, using in situ methods on the FFPE sample can be performed on the sample directly on a slide and then subjected to spatial analysis. Spatial-CUT&Tag (Deng, Bartosovic et al. 2022) assays have recently been established and spatial transcriptomics approaches are established and described in the art, see, e.g., Marshall, Jamie L., et al. “High-resolution Slide-seqV2 spatial transcriptomics enables discovery of disease-specific cell neighborhoods and pathways.” Iscience 25.4 (2022); Pang et al, Histopathology, 84:4, p. 577-587 (2023) doi: 10.1111/his.15093. In some embodiments, the methods herein can comprise isolating nuclei from a FFPE sample prior to performing an assay as described herein, followed by single cell (SC) approaches. Groups have successfully developed SC CUT&Tag first using established SC platforms, including the ICELL8 platform (Kaya-Okur, Wu et al. 2019) and the Chromium platform from 10× Genomics (Wu, Furlan et al. 2021). Of note, other immunotethering-based approaches have also been developed for genomic mapping in SCs (e.g., scChIC-seq (Ku, Nakamura et al. 2019), CoBATCH (Wang, Xiong et al. 2019), scCUT&RUN (Hainer, Boskovic et al. 2019)). Further, multiomic CUT&Tag (e.g., Paired-Tag (Zhu, Zhang et al. 2021), scCUT&Tag-Pro (Zhang, Srivastava et al. 2021)) have been developed. Such approaches can be adapted with the disclosure herein for use with the described methods.

In some embodiments, the transposome comprises a nucleotide barcode sequence. Barcode identifier sequences are known in the art and typically comprise about 6 to 25 nucleotides in length. The barcode sequence and methods of incorporation and use can be as described in International Patent Publication No. WO 2019140082 and International Patent Publication No. WO 2020132388, incorporated herein by reference. Barcoding can alternatively or additionally be incorporated via other ligation strategies, including, for example, splint ligation or sticky ligation, with methods including split-and-pool barcoding. See, e.g., Satz, A.L., Brunschweiger, A., Flanagan, M.E. et al. DNA-encoded chemical libraries. Nat Rev Methods Primers 2, 3 (2022); Quinodoz, S.A., Bhat, P., Chovanec, P. et al. SPRITE: a genome-wide method for mapping higher-order 3D interactions in the nucleus using combinatorial split-and-pool barcoding. Nat Protoc 17, 36-75 (2022). Barcoding can be used as needed to identify the cleaved nucleosomal DNA, for example by sample, individual, or other source identifying information. In some embodiments, barcodes can be included with primers utilized with PCR, for example, indexed primers are described by Buenrostro, J. D. et al. Single-cell chromatin accessibility reveals principles of regulatory variation. Nature 523:486 (2015).

In some embodiments, the method can further comprise evaluating a DNA Integrity Number (DIN) value for the sample e.g., after the isolation of excised tagged DNA from the sample. In some embodiments, a portion of a sample is utilized for evaluating DIN value subsequent to removal of paraffin, with the remainder of the sample being used in the methods described herein. Optionally, one or more steps of the methods of the invention are carried out only when the DIN is greater than or equal to 3. See, e.g., Chougule et al., Comprehensive Development and Implementation of Good Laboratory Practice for NGS Based Targeted Panel on Solid Tumor FFPE Tissues in Diagnostics, Diagnostics, 2022, 12, 1291; doi: 103390/diagnostics 12051291.

In some embodiments, an amount of DNA evaluated in the methods is measured after isolating the DNA fragments. The DNA can be detected by the addition of nucleic acid stains, such as intercalating dyes (e.g., ethidium bromide and propidium iodide, SYBR™ Gold, SYBR™ Green I and SYBR™ Green II, cyanine based dyes), minor groove binders (e.g., DAPI, Hoechst, TOTO-1, indoles, imidazoles, and PicoGreen™) and other stains (e.g., acridine orange, 7-AAD, hydroxystilbamidine (H22845), and LDS 751). Stains may be selected based on desired detection methods. Quantifying DNA can comprise contacting the cleaved or excised fragment with a nucleic acid stain. The methods may comprise quantifying DNA by methods such as spectrophotometry.

In certain embodiments, the methods described herein can comprise identifying transcriptional activity or mapping the location of a protein on chromatin that is indicative of a disease or disorder. The methods described herein can further comprise detecting the amount of mtDNA in a sample, which can further indicate presence of a disease or disorder.

In some embodiments, a method of monitoring a disease or disorder is provided, the method comprising performing a method as described herein from samples obtained at two or more points in time from the same subject, and comparing an amount and/or the genomic location of the targeted protein on chromatin and/or the transcriptional activity on chromatin in each sample to a reference and/or to each other. In some embodiments, the amount of protein or transcription can be indicative of worsening (e.g., increased disease) or improving disease (lessening of the disease). In some embodiments, the reference control may be an aggregate of normal or healthy patients, e.g., one or more patients without the disease. Such reference controls can include healthy population of a particular age, gender, race or other variable. In some embodiments, the reference control comprises comparing a diseased sample to a normal sample from the subject, for example, matched tumor and normal tissue. In an example embodiment, diseased tissue and normal tissue are derived from the same tissue sample, e.g., from the same section or different sections.

In some embodiments, a method of monitoring a disease or disorder comprises determining efficacy of a treatment. In some embodiments, the method comprising performing a method as described herein from samples obtained at two or more points in time from the same subject receiving the treatment and comparing an amount and/or the genomic location of the targeted protein on chromatin and/or the transcriptional activity on chromatin in each sample to a reference and/or to each other. In some embodiments, determining efficacy of a treatment comprises measuring the amount of protein or transcription is indicative of worsening (e.g., increased disease) or improving disease (lessening of the disease) as thereby indicative of efficacy of the treatment. In some embodiments, the differences in the amount and/or genomic location of the targeted protein on chromatin and/or the transcriptional activity on chromatin at the two or more points in time indicate efficacy of a treatment of the disease or disorder in the subject. In addition, the method can monitor disease progression and/or make treatment decisions for subjects based on changes in the amount and/or genomic location of the targeted protein on chromatin and/or the transcriptional activity on chromatin. In an example embodiment, the presently described methods can be used for detection and analysis of amplifications and clonal selection during cancer progression and therapeutic treatment. See, Example 4. In some embodiments, the reference control may be an aggregate of normal or healthy patients, e.g., one or more patients without the disease. Such reference controls can include healthy population of a particular age, gender, race or other variable. The reference control an also comprise healthy tissue from the subject and/or the sample comprising diseased tissue (e.g, tumor). In some embodiments, the first sample is obtained from a subject prior to beginning of treatment. In some embodiments, the second sample is obtained during and/or after treatment.

In some embodiments, a method of diagnosing a disease or disorder in a subject is provided, the method comprising performing a method as described herein on a sample from the subject, and diagnosing the subject as having the disease or disorder based on an amount and/or the genomic location of the targeted protein on chromatin and/or the transcriptional activity on chromatin to thereby diagnose the subject as having the disease or disorder. In some embodiments, the methods comprise correlating the interactions of a target nucleic acid with proteins and/or nucleic acid with a disease state, for example cancer, or an infection, such as a viral or bacterial infection. The profile of the targeted protein on chromatin and/or the transcriptional activity on chromatin can be used to identify binding proteins and/or nucleic acids that are relevant in a disease state such as cancer, for example to identify particular proteins and/or nucleic acids as potential diagnostic and/or therapeutic targets. In an example embodiment, the method can comprise diagnosing a subject with cancer based on the amount and/or the genomic location of the targeted protein on chromatin and/or the transcriptional activity on chromatin which can comprise one or more genes in Table 2.

In some embodiments, the methods described herein can further comprise comparing the amount and/or genomic location of the targeted protein on chromatin and/or the transcriptional activity on chromatin with a control reference. In some embodiments, the reference control comprises comparing a diseased sample to a normal sample from the subject, for example, matched tumor and normal tissue. In an example embodiment, diseased tissue and normal tissue are derived from the same tissue sample, e.g., from the same section or different sections.

In some embodiments, a method of prognosing a disease or disorder in a subject is provided, the method comprising performing a method as described herein on a sample from the subject, and prognosing the disease or disorder in the subject based on the amount and/or genomic location of the targeted protein on chromatin and/or the transcriptional activity on chromatin. A protein involved in transcription regulation can include proteins for chromatin accessibility (e.g., H3K4me2 or RNAPIIS5p). Example phosphoforms of the C-terminal domain of RNAPII that can be used include RNAPII-Ser2, RNAPII-Ser5, RNAPII-Ser7, RNAPII-Ser2/5, or RNAPII-Ser5/7. Chromatin-associated factors, as used herein, are factors that can be found at one or more sites on the chromatin and/or that may associate with chromatin in a transient manner. Examples of low abundance chromatin-associated factors include, but are not limited to, transcription factors (e.g., tumor suppressors, oncogenes, cell cycle regulators, development and/or differentiation factors, general transcription factors (TFs)), ATP-dependent chromatin remodelers (e.g., (P) BAF, MOT1, ISWI, INO80, CHDI), activator (e.g., histone acetyl transferase (HAT)) complexes, repressor (e.g., histone deacetylase (HDAC)) complexes, co-activators, co-repressors, other chromatin-remodelers, e.g., histone (de-) methylases, DNA methylases, replication factors and the like. Such factors may interact with the chromatin (DNA, histones) at particular phases of the cell cycle (e.g., Gl, S, G2, M-phase), upon certain environmental cues (e.g., growth and other stimulating signals, DNA damage signals, cell death signals) upon transfection and transient or stable expression (e.g., recombinant factors) or upon infection (e.g., viral factors). Abundant factors are constituents of the chromatin, e.g., histones and their variants. Histones may be modified at histone tails through posttranslational modifications which alter their interaction with DNA and nuclear proteins and influence for example gene regulation, DNA repair and chromosome condensation. The H3 and H4 histones have long tails protruding from the nucleosome which can be covalently modified, for example by methylation, acetylation, phosphorylation, ubiquitination, sumoylation, citrullination and ADP-ribosylation. The core of the histones H2A and H2B can also be modified. Example chromatin proteins include, but are not limited to, methylated H3K, such as H3K4me2 or H3K4me3, methylated H3K, such as H3K27me3, and acetylated H3K27 (H3K27ac) chromatin proteins.

The disclosed methods can be used for monitoring disease states, such as disease state in an organism, for example a plant or an animal subject, such as a mammalian subject, for example a human subject. Certain disease states may be caused and/or characterized by differential binding of proteins and/or nucleic acids to chromatin DNA in vivo. For example, certain interactions may occur in a diseased cell but not in a normal cell. In other examples, certain interactions may occur in a normal cell but not in diseased cell. Thus, using the disclosed methods, a profile of the interaction can be generated allowing correlation with a disease state. In some embodiments, an interaction profile for a particular disease or disorder state (e.g., infection, cancer, autoimmune disorder), or for a particular subject, subpopulation or population, can be generated using the methods described herein that can be used for diagnosis or prognosis of subjects with a similar interaction profile. Accordingly, aspects of the disclosed methods relate to correlating the interactions of a target nucleic acid with proteins and/or nucleic acid with a disease state, for example cancer, or an infection, such as a viral or bacterial infection. It is understood that a correlation to a disease state could be made for any organism, including without limitation plants, and animals, such as humans.

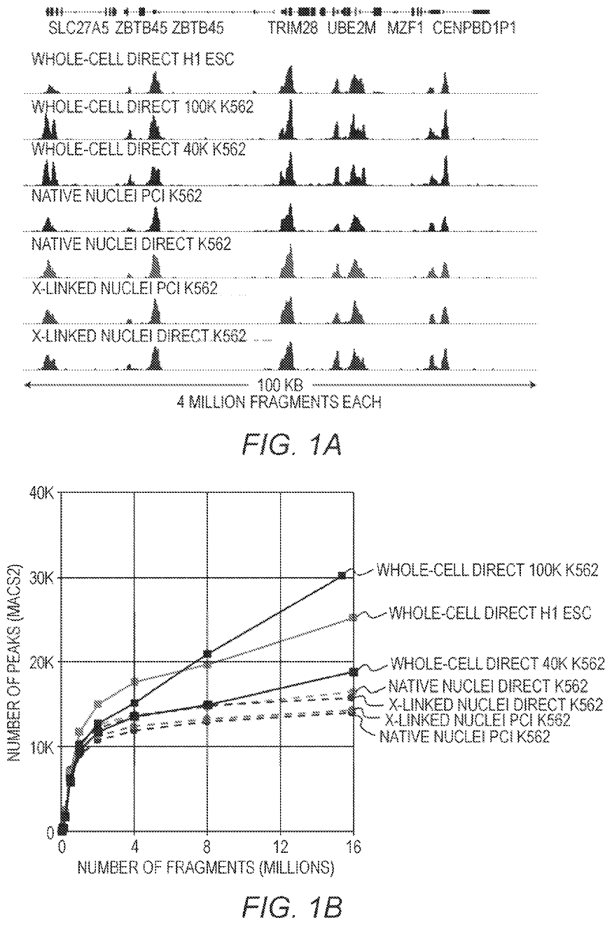

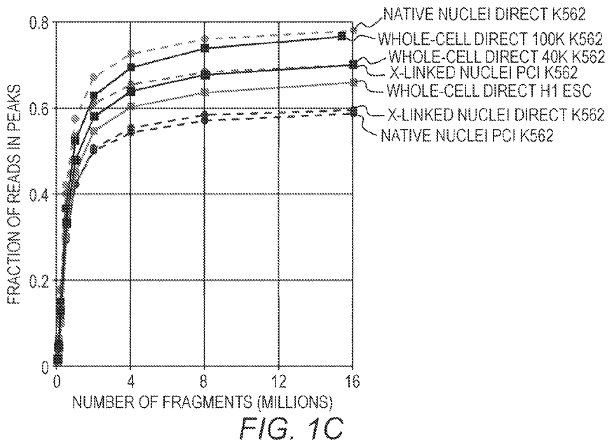

In some embodiments, a method of detecting hypertranscription in a sample is provided, comprising performing a method as described herein, wherein an increased amount of transcriptional activity on chromatin thereby detects hypertranscription in the sample. The method may comprise direct measurements of transcription initiation, elongation, and termination by mapping and quantitating RNAPII to thereby characterize hypertranscription at active regulatory elements. In some embodiments, the method comprises detecting RNAPII at non-coding regions, including, for example, enhancers (e.g., proxy enhancer activation. See, e.g., de Langen P, Hammal F, Guéret E, Mouren J C, Spinelli L, Ballester B. Characterizing intergenic transcription at RNA polymerase II binding sites in normal and cancer tissues. Cell Genom. 2023 Sep. 29;3 (10): 100411. doi: 10.1016/j.xgen.2023.100411 (showing intergenic transcription at RNAPII-bound regions is a novel per-cancer and pan-cancer biomarker and providing an atlas (see FIG. 1, data S1) of intergenic transcription using RNAPII binding sites to connect genomic and transcriptomic data in normal tissues and cancer samples), incorporated herein by reference in its entirety. Hypertranscription, as used herein, refers to a global increase in nascent transcription and can be measured across the genome, mapping hypertranscription at regulatory elements across the genome. In an example embodiment, hypertranscription can be quantified, which can comprise normalizing count differences between tumor tissue sample and normal tissue sample from the same subject and/or same FFPE sample, with an example approach for quantification of hypertranscription is described in Example 4. In an example embodiment, tumor tissue and normal tissue count differences for ENCODE-annotated cCREs is performed, wherein the ENCODE-annotated cCRES can be, for example, promoter, proximal or distal enhancer, or insulator sites,. In some embodiments, replication-coupled histone clusters are used as proxies for cell proliferation to confirm hypertranscription within the samples. In some embodiments, the presently disclosed methods allow application of data mining tools to infer gene regulatory networks. In some embodiments, a peak-caller, for example, SEACR (Meers, M.P., Tenenbaum, D. & Henikoff, S. Peak calling by Sparse Enrichment Analysis for CUT&RUN chromatin profiling. Epigenetics & Chromatin 12, 42 (2019); doi: 10.1186/s13072-019-0287-4), is applied to identify hypertranscribed loci throughout the genome.

In some embodiments, a method of quantifying increases or decreases in RNAPII at one or more loci is provided, the method comprising performing a method as described herein, wherein the first affinity reagent specifically binds to a subunit of the RNAPII complex or a phosphoform of the C-terminal domain of RNAPII, such as RNAPII-Ser2, RNAPII-Ser5, RNAPII-Ser7, RNAPII-Ser2/5, or RNAPII-Ser5/7. See, e.g., Turowski T W, Boguta M. Specific Features of RNA Polymerases I and III: Structure and Assembly. Front Mol Biosci. 2021 May 14; 8:680090; doi: 10.3389/fmolb.2021.680090; Célia Jeronimo, Pierre Collin, François Robert, The RNA Polymerase II CTD: The Increasing Complexity of a Low-Complexity Protein Domain, Journal of Molecular Biology, Volume 428, Issue 12, 2016. Pages 2607-2622. The method can further comprise comparing the results to a control reference. The method may comprise direct measurements of transcription initiation by mapping and quantitating paused RNAPII to thereby characterize hypertranscription at active regulatory elements, such as promoters, enhancers, gene bodies, etc. Methods can comprise quantifying increases or decreases in RNAPII relative to a control reference, for example a known value or range of values indicative of basal levels of RNAPII or amounts or presence in a tissue or a cell or populations thereof, for example a non-diseased (e.g., non-cancerous) state tissue or cell. In an example embodiment, hypertranscription of a cis-regulatory element (cCRE) is measured as the excess of RNAPII-Ser5p in the indicated tumor over normal.

In some embodiments, a method of detecting presence of a protein of interest on chromatin is provided, comprising performing a method as described herein, wherein the first affinity reagent that specifically binds to the targeted chromatin protein is specific for the protein of interest to thereby detect the presence of the protein of interest on chromatin.

The disclosure encompasses methods of detecting an amount of a protein of interest on chromatin, comprising performing a method as described herein, wherein the first affinity reagent that specifically binds to the targeted chromatin protein is specific for the protein of interest to thereby detect the amount of the protein of interest on chromatin.

In some embodiments, the disclosure provides a method of detecting an epigenetic modification on a protein, comprising performing a method as described herein, to determine the presence of the epigenetic modification on the protein. Each of these variations of the methods of the invention can be used, e.g., in diagnosing, prognosing, and/or monitoring a disease or disorder in a subject.

The disclosure also encompasses methods of preparing a library of excised chromatin DNA that is amenable to sequencing on any desired platform. The method comprises the steps described herein.

Compositions that can be used in the methods described herein are also provided. In some embodiments, a composition comprises a deparaffinized and permeabilized FFPE sample containing an RNAPII specific affinity reagent that is linked directly or indirectly to a transposome in low ionic conditions. In some embodiments, a composition comprises a deparaffinized and permeabilized FFPE sample containing a chromatin protein specific affinity reagent that is linked directly or indirectly to a transposome in low ionic conditions.

In another aspect, the disclosure provides a kit of reagents, and optionally instructions, to facilitate performance of the methods described herein. In some embodiments, the kit comprises two or more reagents (e.g., 3, 4, 5, or more) selected from a RNAPII-specific affinity reagent, one or more chromatin protein-specific affinity reagent, a SDS solution, a Triton® X-100 (octyl phenol ethoxylate) solution, a transposase solution, a tagmentation buffer, a cross-linking reversal solution, and amine-functionalized magnetic beads. These reagents are described in more detail above and all embodiments thereof are encompassed by this aspect and are not repeated here in detail. The kit may also comprise a low ionic solution to provide ionic conditions for transposase activity. The kit can optionally include written indicia (for example labels and/or instructions) directing the performance of the method as described herein. Such labeling and/or instructions can include, for example, information concerning the amount, and method of administration, detection and quantification for the assays detailed herein.

Having described the present invention, the same will be explained in greater detail in the following examples, which are included herein for illustration purposes only, and which are not intended to be limiting to the invention.

### EXAMPLES

### Example 1. Epigenomic Analysis of Formalin-Fixed Paraffin-Embedded Samples by CUT&Tag

The prospect of applying chromatin profiling to distinguish regulatory element changes is especially attractive for translational cancer research, insofar as misregulation of promoters and enhancers in cancer can provide diagnostic information and may be targeted for therapy (3). However, there has been limited progress in applying chromatin profiling techniques to FFPEs (4). Although several methods have been developed for chromatin immunoprecipitation with sequencing (ChIP-seq) using FFPEs (5-10), ChIP-seq is not well-suited for small amounts of material that are typically available from patient samples. Furthermore, solubilization of such heavily cross-linked material is extremely challenging, requiring strong ionic detergents and/or proteases in addition to controlled sonication or micrococcal nuclease (MNase) digestion treatments.

Alternatives to ChIP-seq for chromatin profiling include ATAC-seq (11) and enzyme-tethering methods such as CUT&RUN (12) and CUT&Tag (13). Modifications to the standard ATAC-seq protocol were required to make it suitable for FFPEs, including nuclei isolation following enzymatic tissue disruption and in vitro transcription with T7 RNA polymerase (14, 15). The same group also similarly modified CUT&Tag and included an epitope retrieval step using ionic detergents and elevated temperatures, which they termed FFPE tissue with Antibody-guided Chromatin Tagmentation with sequencing (FACT-seq) (16). However, FACT-seq is a 5-day protocol even before sequencing, and the many extra steps required relative to CUT&Tag have raised concerns about experimental variability (4).

A fundamentally different approach to what has been described for FFPE-ATAC and FACT-seq was investigated to determine if it might overcome the obstacles that have thus far been encountered in chromatin profiling of FFPEs. Rather than enzymatically breaking down the tissue for nuclei isolation, only heat and minimal shearing of the tissue were used, then followed our standard CUT&Tag-direct protocol with modifications. This includes applying our Cleavage Under Targeted Accessible Chromatin (CUTAC) strategy, which preferentially yields <120-bp fragments released by antibody-targeted paused RNA Polymerase II (RNAPII) (17, 18). Because of the small size of the fragments released with CUTAC, it is relatively robust to the serious DNA degradation that occurs during cross-link reversal (19), and by attaching to magnetic beads and following the single-tube CUT&Tag-direct protocol experimental variation was minimized. The resulting FFPE-CUTAC profiles could be used to confidently distinguish different mouse brain tumors from one another and from normal brains, identifying potentially key regulatory elements involved in cancer progression.

### Results

### CUT&Tag Streamlined Protocol for Whole Cells

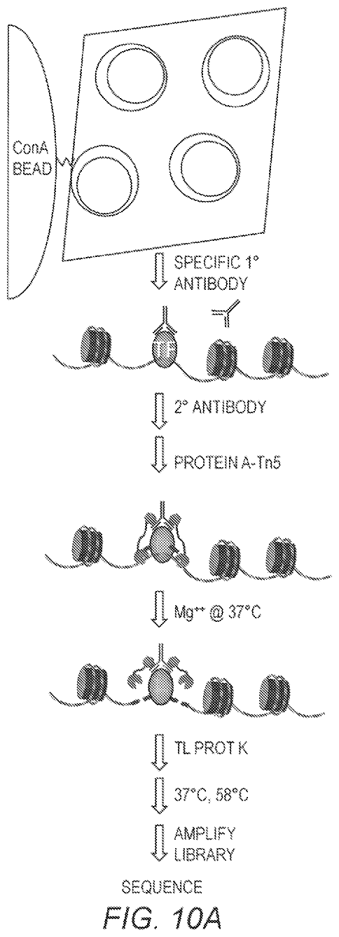

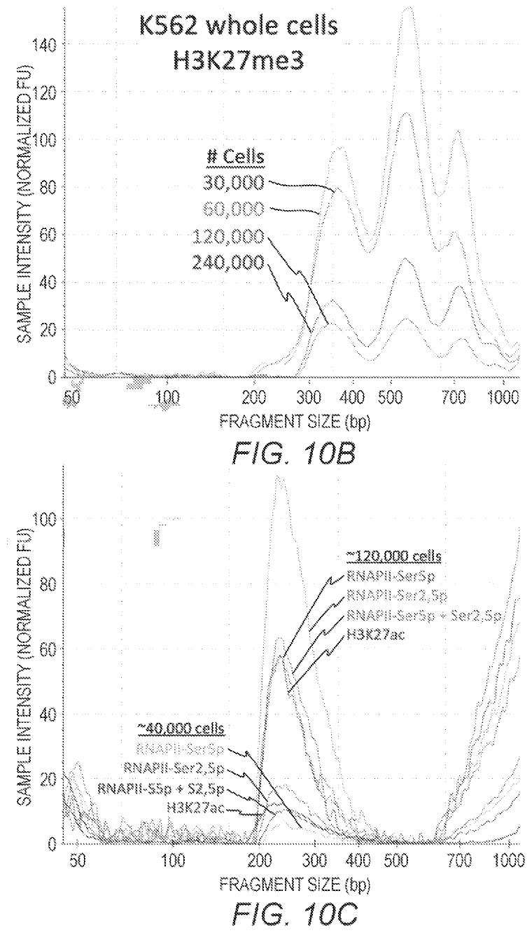

CUT&Tag with DNA purification by addition of SDS/Proteinase K followed by either phenol-chloroform-isoamyl alcohol extraction and ethanol precipitation or SPRI bead binding and elution for PCR were originally introduced (13). Later the protocol was streamlined so that it could be performed in single PCR tubes using a 58° C. incubation in 0.1% SDS followed by excess Triton®-X100, which sequesters the SDS in micelles, allowing efficient PCR (17). However, this CUT&Tag-direct method was only suitable for up to ~50,000 nuclei, as more material was found to inhibit the PCR. To make CUT&Tag-direct applicable to whole cells, 0.05% Triton®-X100 was included in all buffers from antibody addition through tagmentation, which maintains cells permeable without disrupting nuclei and improves bead behavior. The concentration of SDS was also increased and thermolabile Proteinase K included in the fragment release buffer. After digestion at 37° C. and inactivation at 58° C., the SDS is quenched with excess Triton®-X100 and the material is subjected to PCR, resulting in high yields with 30,000-60,000 cells (FIGS. 10A-10B). When applied to the H3K4me3 promoter mark, this modified CUT&Tag-direct protocol for native whole cells resulted in representative profiles that match those of native or fixed nuclei using either the original organic extraction method or CUT&Tag-direct (FIG. 1A). Based on MACS2 peak-calling and Fraction of Reads in Peaks (FRiP), slightly more peaks called and similar FRiP values for up to at least 100,000 native whole cells were obtained using the modified protocol (FIGS. 1B-1C), obviating the need to purify nuclei for CUT&Tag-direct and AutoCUT&Tag (20).

### Temperature-Dependent Permeabilization of FFPE Sections for CUTAC

The difficulty of performing CUT&Tag-direct on FFPEs is exacerbated not only by the severe chromatin damage caused by heavy formalin fixation but also by the large amount of cross-linked intra- and inter-cellular material that cells are embedded in. Both the FFPE-ATAC and FACT-seq methods require lengthy digestion with collagenases and hyaluronidases followed by needle extraction and straining liberated nuclei for processing. It was reasoned that such harsh treatments might not be necessary if the cells can be permeabilized sufficiently, and we were encouraged to attempt this approach by the fact that deparaffinized 10 micron FFPE samples on slides are routinely permeabilized for cytological staining with antibodies (1). Also, there has been recent progress in preventing the most severe DNA damage to FFPEs by careful attention to buffer and heating conditions (19). Accordingly, manual shattering of 10 micron FFPE sections from tumor and normal mouse brains were performed by dicing and scraping the tissue off of slides with a razor blade followed by forcing the solution twenty times through a 22-gauge needle. It was found that the Concanavalin A (ConA) beads used for standard CUT&Tag bound sufficiently well to shattered FFPE fragments regardless of whether they had been prepared from samples deparaffinized using a xylene or a mineral oil procedure. This meant that all steps from antibody addition through to PCR could be performed on FFPEs following the same CUT&Tag-direct protocol used for nuclei and whole cells. In addition, the toughness of FFPE shards allowed for hard vortexing and centrifugation steps that would result in lysis of ConA bead-bound cells or nuclei.

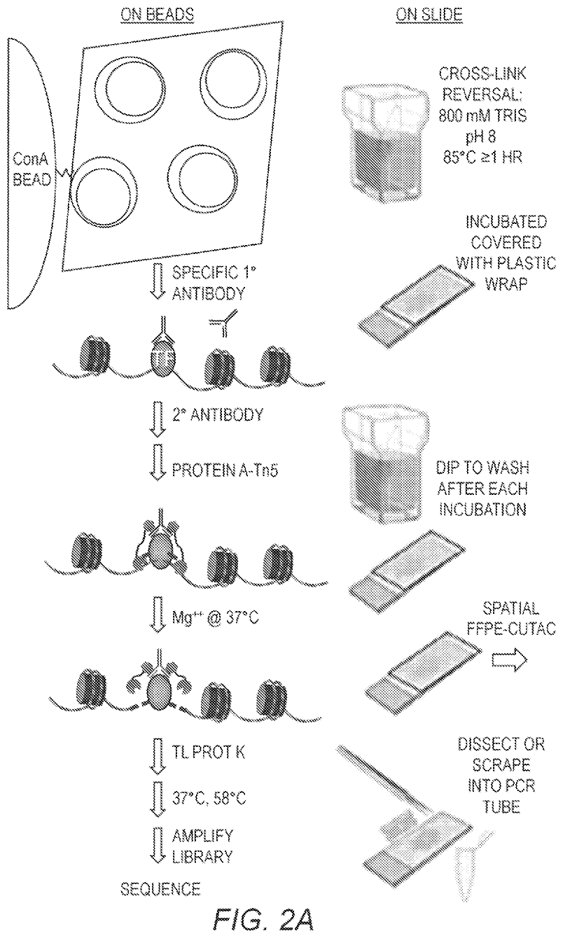

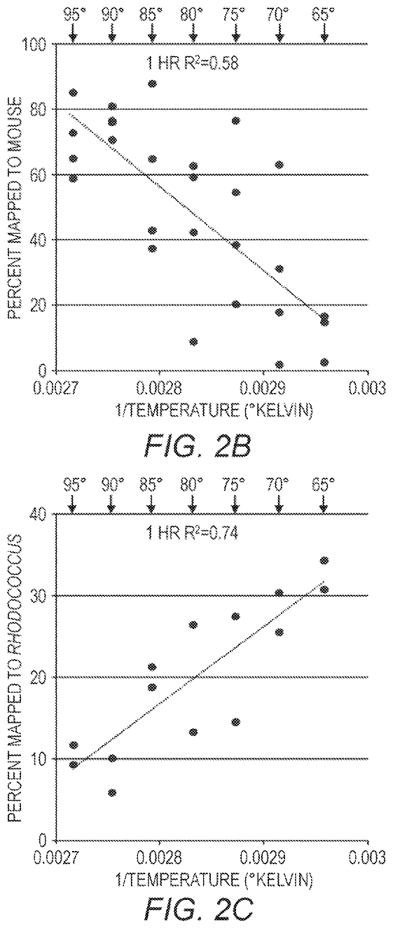

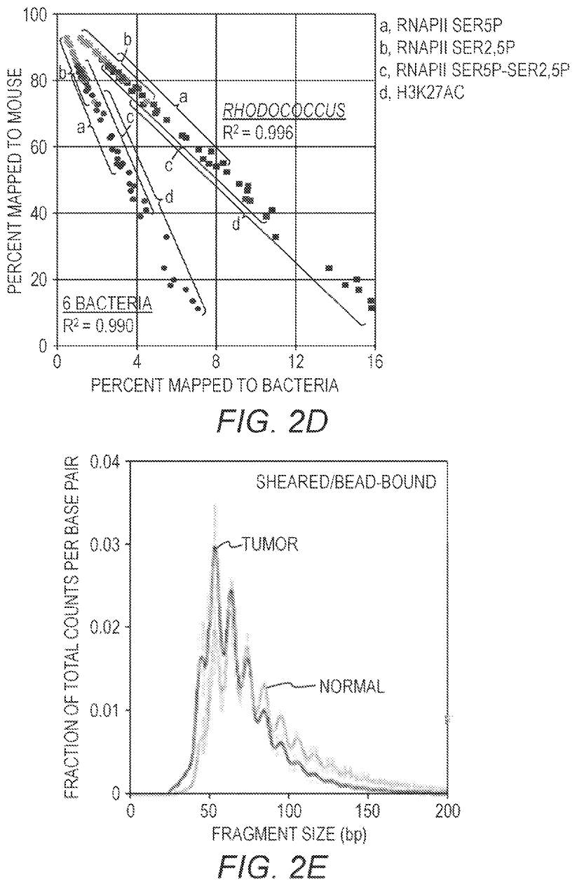

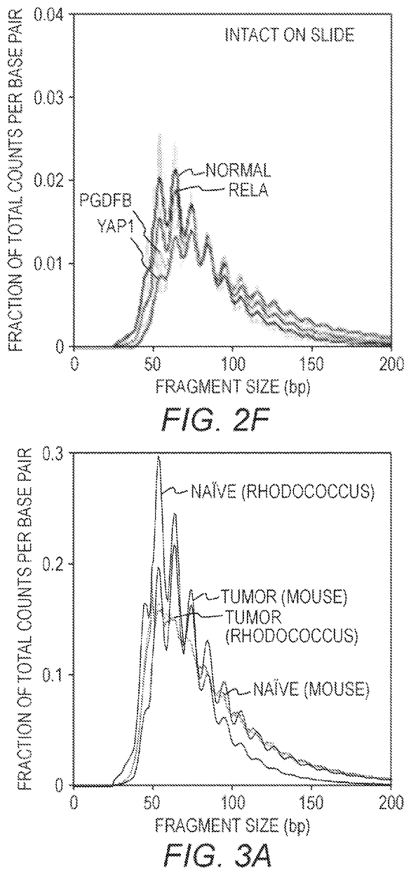

Formaldehyde cross-links are reversed by incubation at elevated temperatures. A relationship between cross-link reversal and incubation temperature has been determined to follow the Arrhenius equation (21). Typical ChIP-seq, CUT&RUN and CUT&Tag protocols recommend cross-link reversal at 65° C. overnight in the presence of proteinase K and SDS to simultaneously reverse cross-links and deproteinize. However, the much more extreme formaldehyde treatments that are used in preparing FFPEs have required incubation temperatures as high as 90° C. for isolation of PCR-amplifiable DNA for whole-genome sequencing (19, 22, 23). High temperatures also contribute to epitope retrieval for ChIP-seq (5-10) and FACT-seq (16), and for cytological staining one protocol calls for epitope retrieval at 125° C. at 25 psi in a pressure cooker (24). To optimize the temperature of incubation for DNA recovery and epitope retrieval for CUT&Tag on FFPE samples from mouse brain tumors, shattered FFPEs were incubated at temperatures ranging from 65° C. to 95° C. before ConA bead and antibody additions. Modified CUT&Tag-direct using low-salt tagmentation was performed with RNAPII Ser-5p and/or Ser-2,5p and H3K27ac antibodies to enrich for sites of chromatin accessibility (CUTAC). This resulted in capillary gel profiles consisting almost entirely of library inserts averaging ~60 bp (FIG. 10C). Upon DNA sequencing, the fraction of fragments that mapped to the mouse genome showed a strong temperature dependence, where the highest temperatures (90-95° C.) showed the highest fraction of mapping to the mouse genome (75%), and the lowest temperatures (65-70° C.) showed the lowest fraction (13%). Temperature dependence followed the Arrhenius equation (FIG. 2A), which based on previous work (21) suggests that the limiting factor in tagmented fragment recovery is reversal of cross-links.

### Rhodococcus Contamination Provides a Calibration Standard

The identity of CUT&Tag-generated fragments that did not map to the mouse genome was explored. Using BLASTN against nucleotide sequences in Genbank it became apparent that there was a single species that consistently rose to the top of the list for all samples, the gram-positive bacterium Rhodococcus erythropolis. Mapping fragments to the R. erythropolis genome, it was found that the entire genome was represented as expected if this species is a major contaminant of the mouse brain FFPEs in our study. Consistent with this interpretation, a high-temperature dependence of fragment release opposite that for mouse was found (FIG. 2B), consistent with Rhodococcus fragments competing with mouse fragments in PCR. A near-perfect anti-correlation between the fraction of fragments mapped to mouse and the fraction mapped to R. erythropolis (R2=0.996, n=59) across all antibodies and experiments was found, with Rhodococcus accounting for ~1-15% of the fragments (FIG. 2C). As bacterial DNA is not chromatinized it is not likely to be as well protected from melting as is mouse DNA, and so will not serve as a substrate for Tn5 tagmentation, which could account for the reduction in Rhodococcus contamination with increasing temperature.

To obtain a broader representation of species contaminating our FFPEs, BLASTN searches of the RefSeq Genome Database were performed using a sample of 300 multiply represented 50-bp reads not aligning to the mm 10 build of the mouse genome. A search of the bacterial genome subset returned hits to diverse species for 208 species, which implies that a minimum of 2/3 of the unmapped reads were bacterial in origin. Although no other bacterial species were nearly as abundant as R. erythropolis, summing the fragment counts mapped to the six most frequently represented other species accounted for ~0.5-7% of the fragments and showed similar anti-correlations to mouse (R2=0.990, FIG. 2C). Efficiency was highest for RNAPII Ser2,5p (85% mouse, 2.5% Rhodococcus) and lowest for H3K27ac (38% mouse, 11% Rhodococcus). The lower efficiency of the histone modification, and our observation that this protocol was not successful for H3K4me2 and H3K4me3, may suggest that lysine-rich histone tails are more subject to formaldehyde adduct and cross-linking damage than the C-terminal domain of Rpb1, which consists of 52 copies of a lysine-free YSPTSPS heptamer.

What is the source of Rhodococcus and other bacterial contamination in our FFPEs, which derive from multiple FFPE sample preparations over a 2-year span? R. erythropolis isolates have been found to use paraffin wax as their sole carbon source (25). The species has also been proposed as a biodegrader for removing the paraffin wax that remains on the inner surfaces of oil tanker holds after they are emptied (26). It was inferred that most of the DNA fragments that do not map to mouse are derived from the paraffin used in embedding, with an advantage during PCR over the tissue derived DNA in not having been subjected to formalin fixation. The inverse relationship between mouse and Rhodococcus and other bacterial DNAs in FFPE samples makes Rhodococcus contamination an ideal calibration standard, because the two genomes are already present in the initial FFPE samples. This is unlike spike-ins used routinely for calibration of epigenomic and transcriptomic profiling, which require a mixing step that inevitably introduces stochastic errors. The near-perfect anti-correlation seen for these two genomes in different samples was interpreted as reflecting a very uniform distribution of contamination for slides prepared at different times.

### Global Accessibility in FFPEs is Expanded in Mouse Brain Tumors

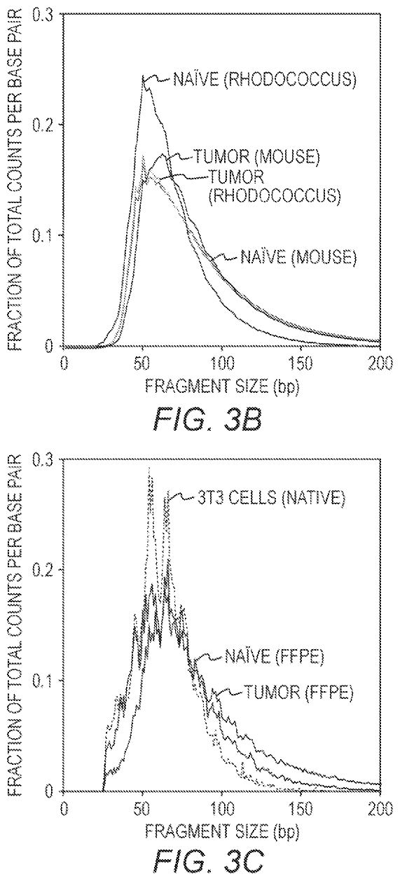

For fragments mapping to the mouse genome, length distributions showing 10-bp periodicities typical of CUT&Tag but peaking at ~60 bp (FIG. 3A) were observed, similar to what is seen for the subnucleosomal peaks observed in CUT&Tag experiments. Our FFPEs included brain tumors driven by RelA, Pdgfb or Yap1 transgenes or were naïve brain samples. When the fragment length distributions were plotted, high concordance between the different tumor samples were observed, but a marked difference between tumors and naïve brains, where the length distribution was shifted with more longer fragments in tumors relative to naïve brains (FIG. 3A). In contrast, the two overall length distributions of Rhodococcus DNA fragments from the same tumor and naïve samples almost perfectly superimposed. The fragment length distribution for tumor samples is similar to that of the non-chromatinized Rhodococcus genome for >100-bp fragments when the 10-bp periodicity that is characteristic of Tn5 tagmentation is smoothed (FIG. 3B). Thus, the genome in tumor cells appears to be more accessible than that in naïve cells. However, this shift to a longer fragment distribution for tumors is also seen for mitochondrial DNA from the same samples when compared to either naïve brain or CUT&Tag mitochondrial DNA profiles from native 3T3 fibroblasts (FIG. 3C). The longer fragment distribution for both nuclear and mitochondrial tumor samples relative to naïve samples were attributed to a tissue-wide change in DNA accessibility that is covalently fixed by formaldehyde cross-linking. Interestingly, both Rhodococcus and mouse mitochondrial fragments from FFPEs displayed much weaker 10-bp periodicity relative to mouse FFPE nuclear and unfixed mouse mitochondrial fragments, respectively, suggesting that the reduction in periodicity seen for DNA unimpeded by nucleosomes (bacterial and mitochondrial) is the result of DNA damage caused by fixation. The strong periodicity seen for mouse CUT&Tag profiles relative to non-chromatinized DNA of bacteria and mitochondria might reflect partial protection from unreversible formaldehyde fixation damage by RNAPII and other chromatin regulatory complexes characteristic of open chromatin (27).

### FFPE-CUTAC Produces High-Quality Maps of Active Chromatin

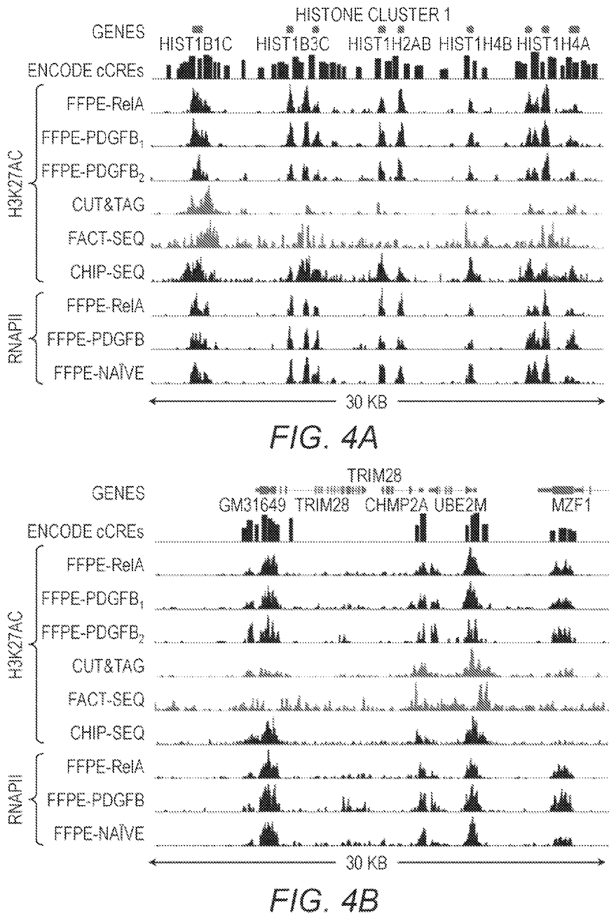

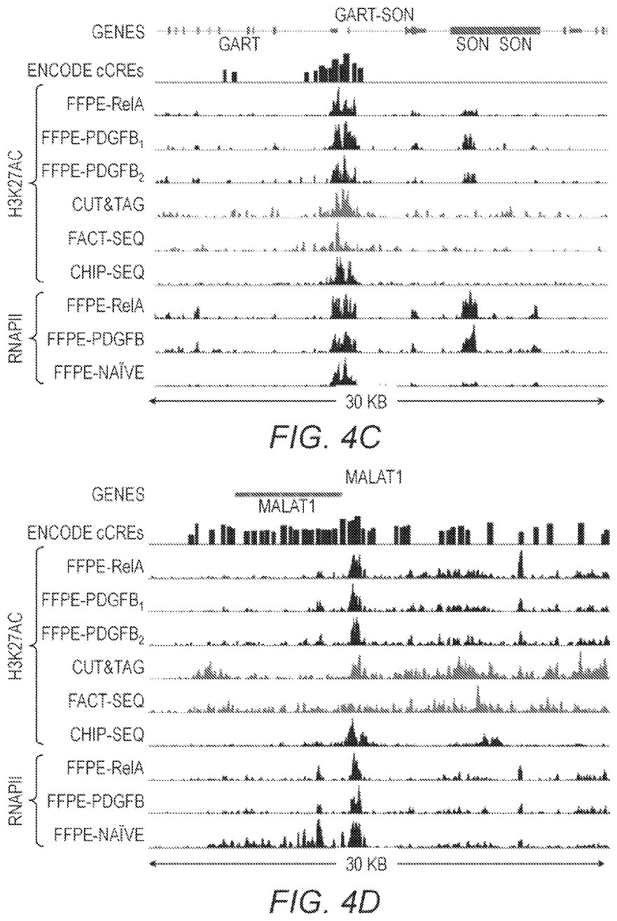

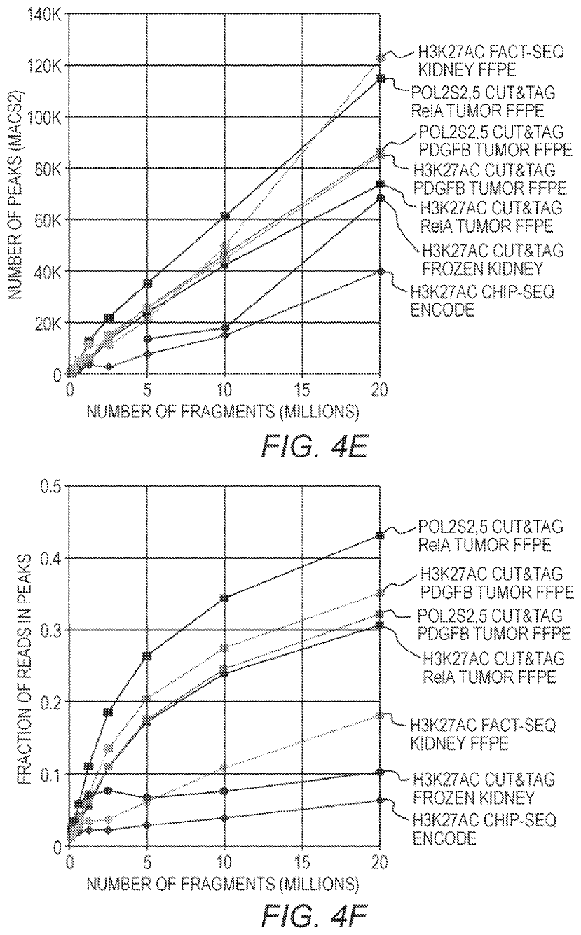

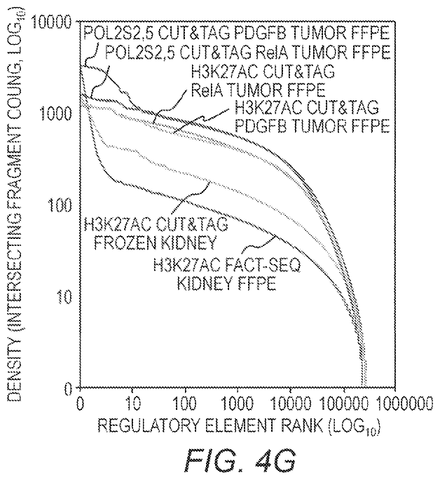

To evaluate the accuracy and data quality of FFPE-CUTAC derived from mouse brain tumors, tracks between FFPE-CUTAC and FACT-seq or standard CUT&Tag from the same study (16) using the same H3K27ac antibody (Abcam cat. no. 4729) were compared. Because of differences in cell types, brain tumors in this study and kidney or liver in the FACT-seq study, comparisons of tracks to housekeeping genes that are expected to be similarly expressed in all cell types were limited. Based on visual inspection of tracks from representative regions of the mouse genome, it is evident that H3K27ac CUTAC profiles show much cleaner profiles than those obtained using FACT-seq, with higher sensitivity than the data obtained for CUT&Tag controls of frozen mouse kidney (FIGS. 4A-4D). Likewise, clean profiles were also seen for RNAPII-Ser2,5p FFPE-CUTAC, where RNAPII-Ser2 phosphate marks elongating and RNAPII-Ser5 phosphate marks paused RNAPII.

For a systematic analysis of data quality, peaks were called using MACS2 and compared the number of peaks called and FRiP values. Both H3K27ac and RNAPII-Ser2,5p FFPE-CUTAC on RelA-and Pdgfb-driven brain tumors showed much better sensitivity based on number of peaks called and much higher FRiP values than either H3K27ac CUT&Tag on frozen kidney or FACT-seq on FFPEs (FIGS. 4E-4G).

To determine the degree to which FFPE-CUTAC profiles capture regulatory elements, we took advantage of the Candidate cis-Regulatory Elements (cCRE) database generated by the ENCODE project, which called putative regulatory elements from all tissue types profiled. The 343,731 elements in the cCRE mouse database used were based mostly on DNAseI-seq (also H3K4me3 and CTCF ChIP-seq), thus providing a comprehensive standard for FFPE-CUTAC performance, insofar as CUTAC profiles correspond closely to both ATAC-seq and DNAseI-seq profiles (17). For each dataset cCREs were rank-ordered based on normalized counts spanned by each element, which were plotted as a log-log cumulative curve, where a higher curve indicates better performance in distinguishing annotated sites from background. By this benchmark, both H3K27ac and RNAPII-Ser2,5p FFPE-CUTAC brain datasets outperformed both FACT-seq on FFPEs and CUT&Tag on unfixed frozen kidney (FIG. 4G). In conclusion, the FFPE-CUTAC protocol provides high quality data, even when compared to ordinary CUT&Tag.

### RNAPII FFPE-CUTAC Profiles Distinguish Brain Tumors

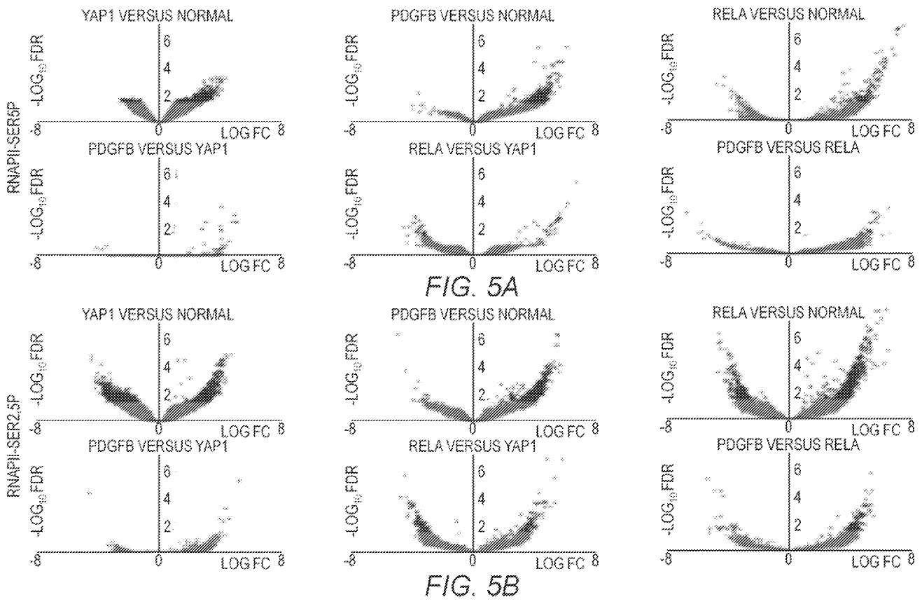

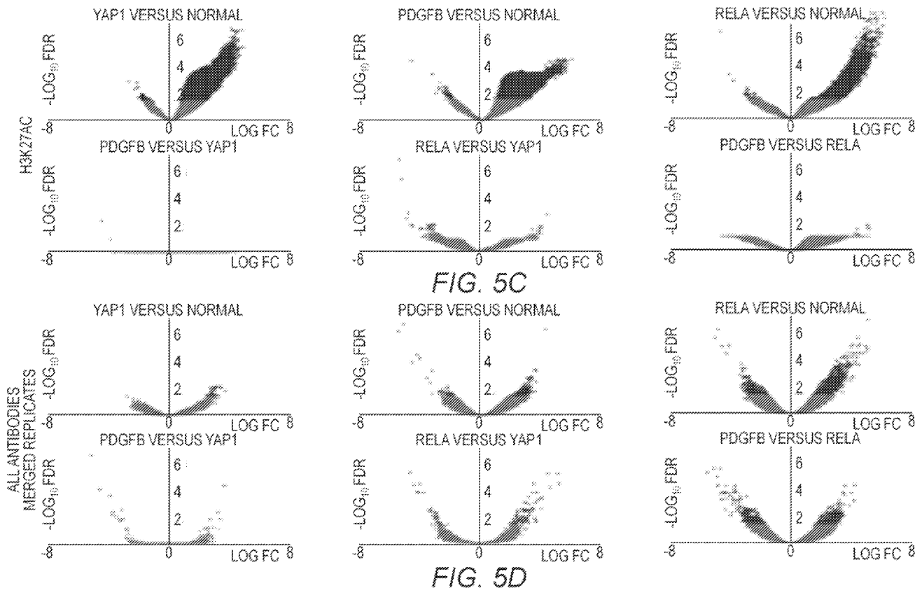

Nearly all strong peaks seen for H3K27ac and RNAPII-Ser2,5p FFPE-CUTAC corresponded to putative regulatory elements from the cCRE database, with concordance between FFPE-CUTAC, FACT-seq and ChIP-seq (FIGS. 4A-4D). To identify tumor-specific candidate regulatory elements pairwise comparisons were performed between three different mouse brain tumors (YAP1—, PDGFB- and RELA-driven tumors) and normal mouse brains. For each of the 343,731 cCREs the normalized counts spanned by the cCRE and performed pairwise comparisons over all cCREs were averaged with Voom/Limma42, an Empirical Bayes algorithm, which uses the other datasets as pseudo-replicates to increase statistical confidence. This approach was applied to datasets from multiple FFPE-CUTAC experiments using antibodies against RNAPII-Ser5p, RNAPII-Ser2,5p and H3K27ac. Far more significant differences were observed for comparisons between tumors and normal brains than between tumors, with more increases than decreases in tumors relative to normal brains (FIGS. 5A-5C and Table 1). For example, using RNAPII-Ser5p, there were 10,321 cCREs that differed between YAP1 and normal brain, 518 between PDGFB and normal brain, and 190 between RELA and normal brain at a False Discovery Rate (FDR)=0.05, but only 10-63 cCREs that differed in pairwise comparisons between the three tumors (FIG. 5A and Table 1, part a). Compared to normal brain, 92-99% of the differences were increases in the tumors. Approximately similar results were obtained using RNAPII-Ser5p (FIG. 5B and Table 1, part b). For H3K27ac, the number of cCREs that increased was more extreme, with nearly half of the 343,371 cCREs significantly increased at the FDR=0.05 level (FIG. 5C and Table 1, part d). These results demonstrate that FFPE-CUTAC using antibodies against RNAPII or H3K27 marks distinguishes between the tumors and the normal brain samples with nearly all significant differences representing increases for the three tumors over normal brain.

As FFPE-CUTAC data quality is very similar between RNAPII-Ser2,5p and H3K27ac (FIGS. 4A-4G), the conspicuous sensitivity differences in pairwise comparisons (FIGS. 5A-5C and Table 1) was attributed in part to the larger number of H3K27ac samples that Voom/Limma used for pseudo-replicates in calculating FDR. To balance the contribution of samples from each genotype, datasets were merged from multiple FFPE-CUTAC experiments for each antibody (RNAPII-Ser5p, RNAPII-Ser2,5p or H3K27ac) or antibody combination (RNAPII-Ser5p+RNAPII-Ser2,5p), then down-sampled to the same number of mapped fragments for each genotype. The three tumor and one normal genotype, each represented by four different antibodies or antibody combination, were compared pairwise with Voom/Limma. The most differences were observed between RELA and Normal (1,657) and between RELA and PDGFB (607) and the fewest differences between PDGFB and YAP1 (17) (FIG. 5D). In conclusion, FFPE-CUTAC can distinguish tumors from one another and from normal brains based on differences in cCRE occupancy of active RNAPII and H3K27ac marks.

### Increases in Paused RNAPII Pinpoint Regulatory Element Differences

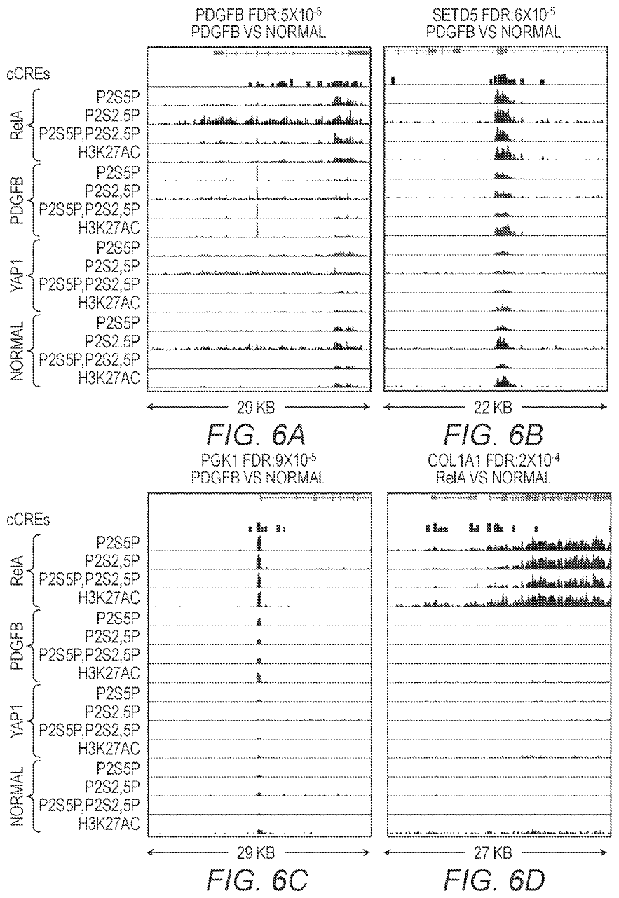

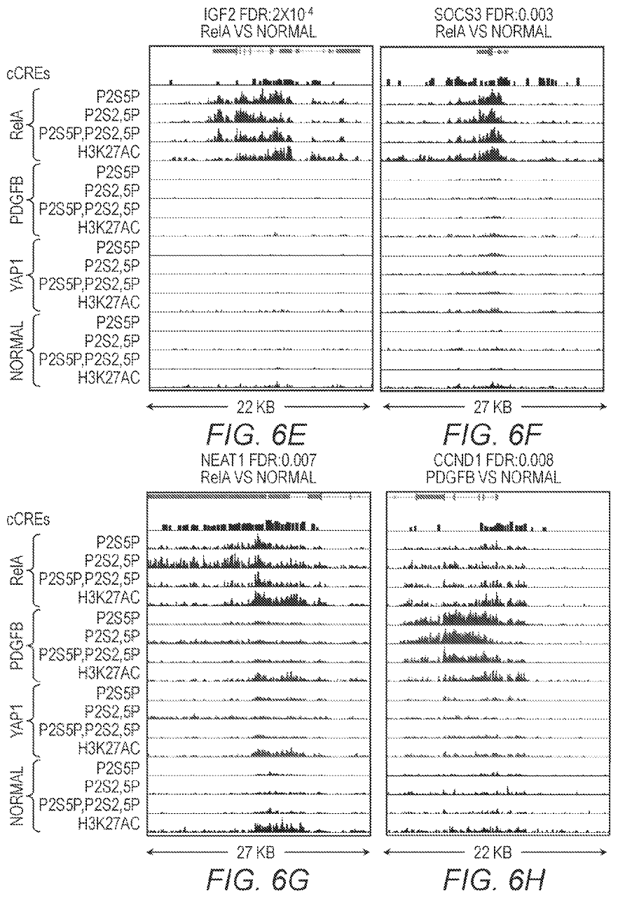

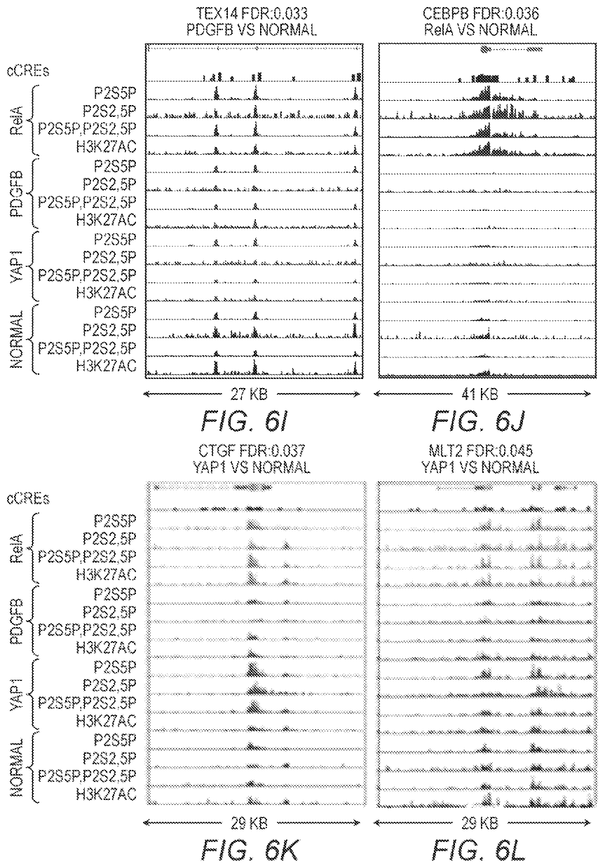

The most significant difference in all RNAPII-Ser5p comparisons is between the Pdgfb-driven tumor and naïve brain (FIG. 6A), seen as a sharp peak a coding exon of the Pdgfb gene (FDR=5×10⁻⁵). This example serves as an internal control, as it corresponds to the virally expressed PDGF-beta growth factor coding region that drives the tumor, even though this sample contained both normal brain and tumorous tissue. The next two most significant differences (FIGS. 6B-6C), which are also from the Pdgfb-driven tumor and naïve comparison, display clear differences between the tumors, with the RelA-driven tumor showing a high signal over the cCRE and the Yap1-driven tumor showing low signal. Even more striking differences between tumors are seen for the next two most significant differences (FIGS. 6D-6E), where the RelA-driven tumor shows a strong signal but there is no perceptible signal in the region for naïve, Pdgfb-driven and Yap1-driven samples. Conspicuous tumor-specific differences are also seen for four of the five cCREs with the highest signals with FDR <0.05 (FIGS. 6F-6J).

The most significant and highly expressed summit differences between tumors and naïve brain identify loci that have been reported as implicated in tumor progression. Among these are the SET domain-containing 5 (Setd5) promoter (FIG. 6B) (29), the phosphoglucokinase (Pgk1) promoter (FIG. 6C) (30), the collagen type 1 alpha 1 (collal) promoter (FIG. 6D) (31), the bidirectional promoter of the insulin growth factor 2 (Igf2) (FIG. 6E) (32), an intronic enhancer in the suppressor of cytosine signaling 3 (Socs3) gene (FIG. 6F) (33), the promoter of the nuclear paraspeckle assembly transcript 1 (Neat1) long non-coding RNA gene (FIG. 6G) (34), a proximal enhancer of the cyclin D1 (Ccndl) gene (FIG. 6H) (35), the C/EBPB promoter (FIG. 6J) (36), the connective tissue growth factor (Ctgf) promoter (FIG. 6K) (37) and an intronic enhancer of the metallothionien 2A (Mt2a) gene (FIG. 6L) (37). Whereas the Testis Expressed 14 (Tex14) gene has not been reported to be implicated in cancer, this is the only one of the top 12 genes in which the tumor/naïve differences were inconspicuous (FIG. 6I), consistent with the supposition that increases in paused RNAPII at enhancers or promoters of the other 11 genes are associated with tumor progression.

FFPE-CUTAC Distinguishes Tumor from Normal Tissue within the Same FFPE

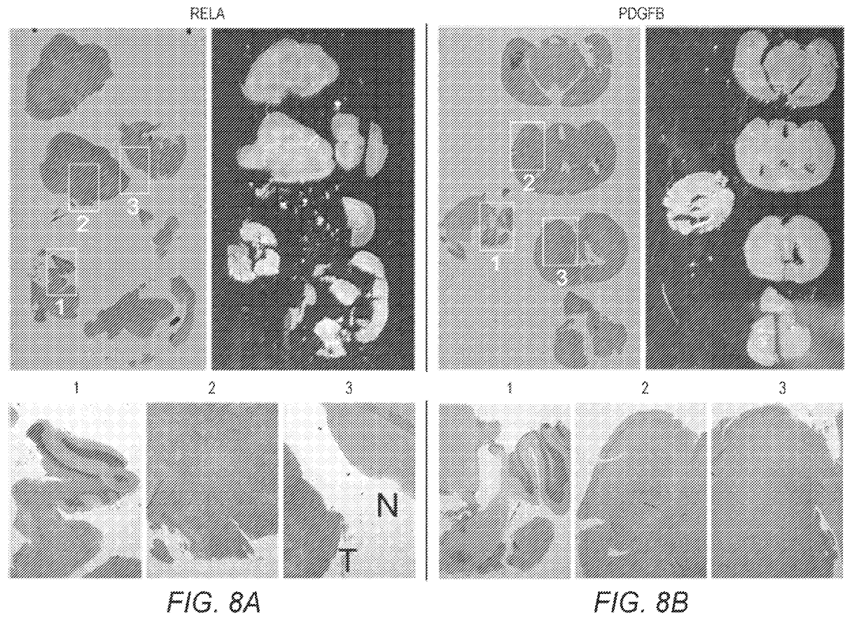

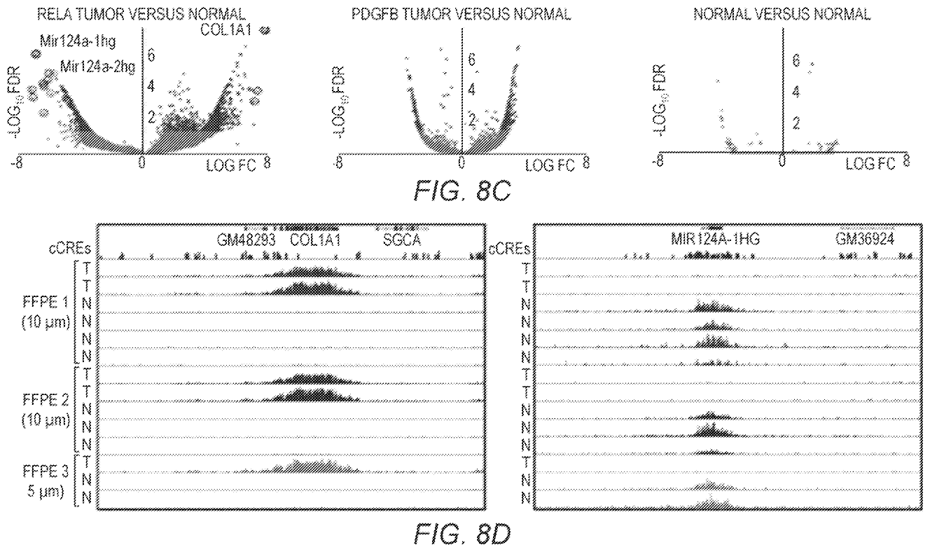

On-slide FFPE-CUTAC (FIG. 2A) provided us with the opportunity to compare tumor with normal tissue on the same slide. For this analysis ZFTA-RELA gene fusion-driven ependymomas (FIG. 8A) were used which are relatively large and cytologically distinct, whereas PDGFB-driven gliomas (FIG. 8B) are more diffuse. On-slide FFPE-CUTAC were performed through tagmentation and manually harvested 6 sections from a single RELA slide and 7 sections from a single PDGFB slide separately into PCR tubes. After sequencing, Voom/Limma analysis was performed comparing the sections identified cytologically as mostly tumor to sections identified as mostly normal. Results for RELA were very similar to those obtained comparing tumor to normal brains, whereas results for PDGFB showed fewer significant CCREs at FDR=0.05 (FIG. 8C). Similar results were obtained with twoother RELA slides, where the top upregulated cCRE was within the Colla gene (FIG. 8D), which was also the top RELA-versus-Normal hit in multiple-slide comparisons (FIG. 5D). Interestingly, the top down-regulated gene in both replicate slides, Mir124a-1 hg, is a microRNA methylation marker locus for Helicobacter pylori infection that correlates with gastric cancer driver gene methylation⁵³. The entire locus is embedded in a cluster of 27 cCREs, and all replicates show a broad RNAPII signal in normal tissue but not RELA-driven tumor encompassing the entire cluster (FIG. 8D). Indeed, the top 10 down-regulated cCREs are either Mir124a-hg1 or Mir124a-hg2 and these together with the next down-regulated cCRE, which is over the Mir670 microRNA locus, account for 15 of the top 25 down-regulated cCREs. In contrast, these genes are far down RNA-seq list ranked by false discovery rate, as Mir124a-1 hg ranks 9,913, Mir124a-2 hg ranks 6,045 and Mir670 ranks 21,262 of 23,551 annotated mouse genes.

FFPE-CUTAC Distinguishes Tumors from Normal Liver

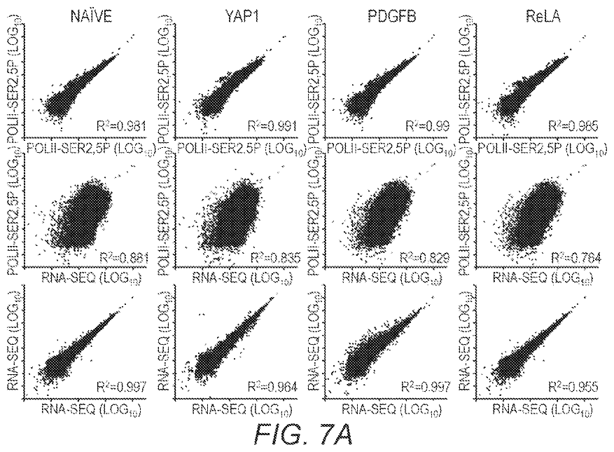

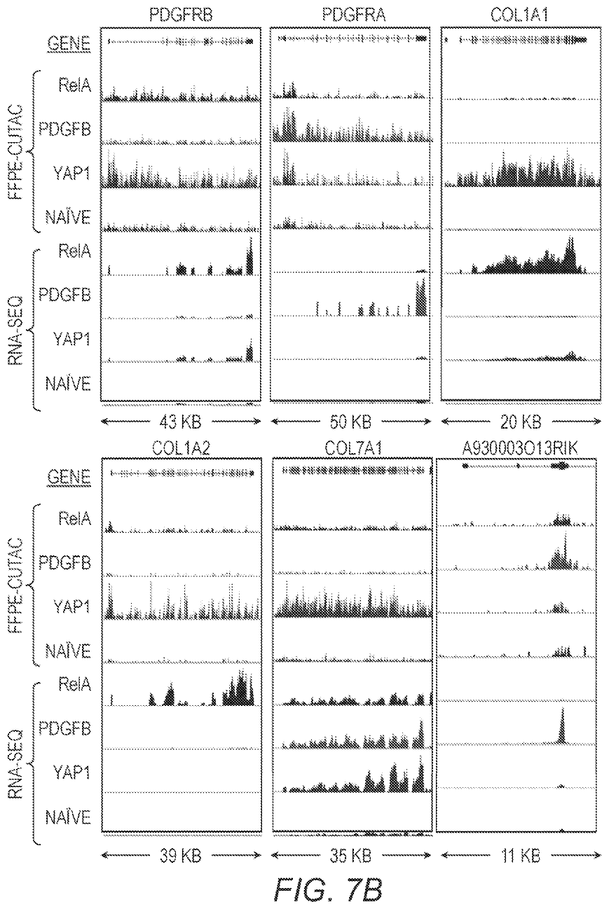

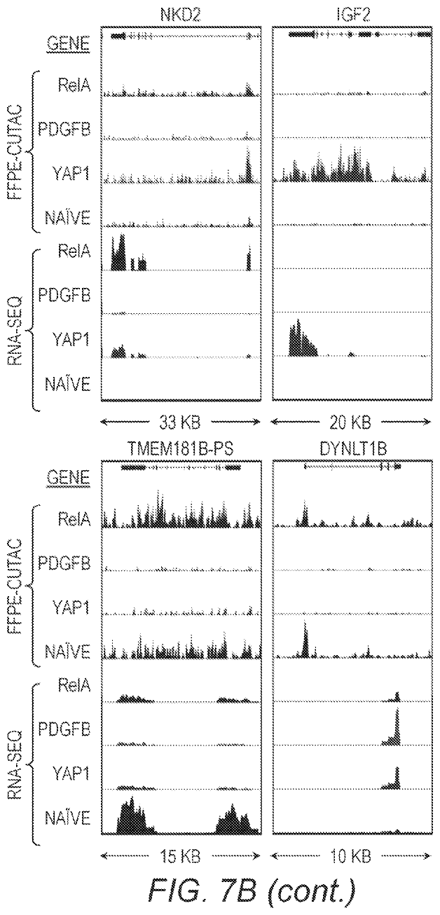

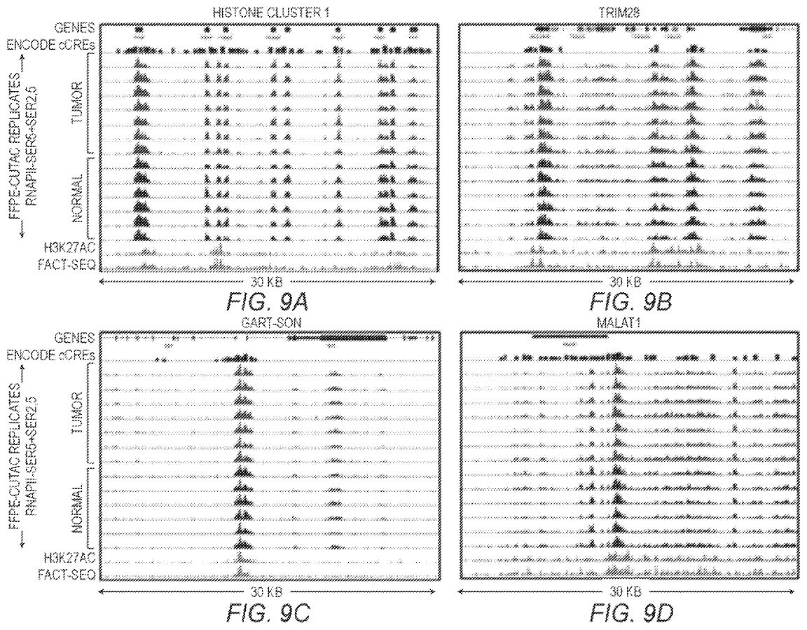

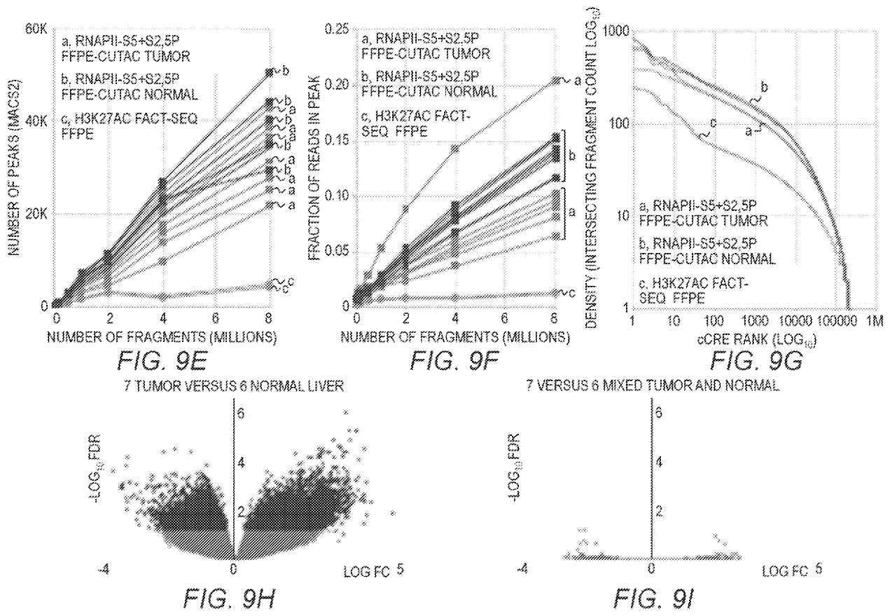

To test whether our results with mouse brain FFPEs generalize to a very different tissue type, FFPE-CUTAC was performed using FFPE sections prepared from intrahepatic cholangiocarcinoma tumors and normal liver. FFPE sections were used that had been fixed in formalin for 7 days and after deparaffinization were incubated at 90° C. in cross-link reversal buffer for 8 hours and incubated with a 50:50 mixture of RNAPII-Ser5p and RNAPII-Ser2,5p antibodies, each at 1:50 concentration. Highly consistent results were obtained for samples ranging from 10% to 50% of a section (~30,000-150,000 cells), with clean peaks over housekeeping genes for both liver tumor and normal liver (FIGS. 7A-7D). As was the case with brain tumor and normal tissues fixed in formalin for 2 days, the number of peaks and fraction of reads in peaks (FRiP) were much higher than those from FACT-seq FFPE livers (FIGS. 9E-9F) and overlap with cCREs was also much higher when down-sampled to the same number of fragments (FIG. 9G). Finally, volcano plots revealed net increases in cCRE RNAPII occupancy both in fold-change and FDR for liver tumors relative to normal livers, similar to what was observed in comparing brain tumors to normal brains (FIGS. 8H-81). In conclusion, FFPE-CUTAC provides high-quality for FFPEs from diverse tissue types.

### Comparison Between FFPE-CUTAC and Standard RNA-Seq on Transgene-Driven Brain Tumors

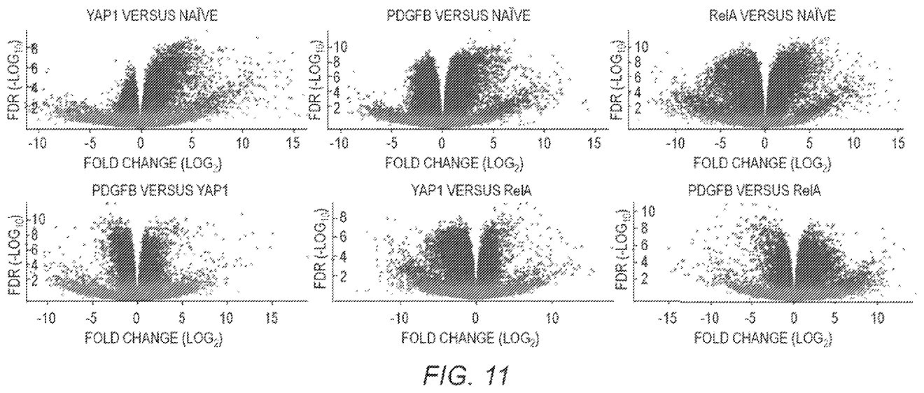

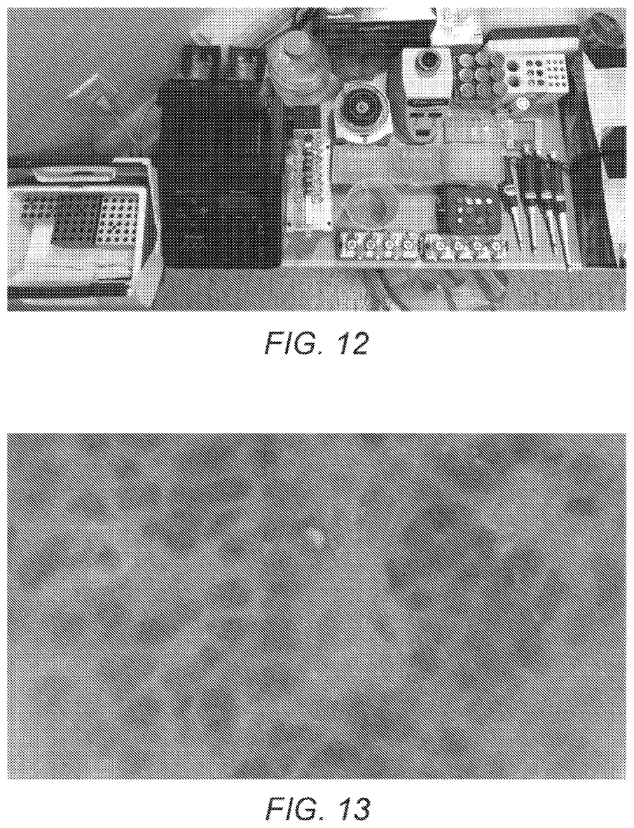

The murine brain tumor lines that were used in the study have served as models for the study of de novo ependymoma tumorigenesis (38-40), with high-quality RNA-seq data available. To do an unbiased comparison between FFPE-CUTAC regulatory elements and processed transcripts mapped by RNA-seq, it was first determined whether there is sufficient overlap between cCREs and annotated 5′-to-3′ genes to fairly compare these very different modalities. Specifically, the 343,731 cCREs average 272 bp in length, accounting for 3.4% of the Mm 10 build of the mouse genome, whereas the 23,551 genes in RefGene average 49,602 bp in length, with an overlap of 54,062,401 bp or 2.0% of Mm10. In other words, the 5′-to-3′ span of mouse genes on the RefGene list should capture all of the RNA-seq true positives and almost 60% (2.0/3.4×100%) of the cCREs. With most cCREs overlapping annotated mouse genes, one can directly compare FFPE-CUTAC fragment counts to RNA-seq fragment counts by asking how well they correlate with one another over genes. Whereas FFPE-CUTAC replicates and RNA-seq replicates are very strongly correlated to a similar extent, with “arrowhead” scatterplots (R2=0.955-0.997), similar scatterplot comparisons between FFPE-CUTAC and RNA-seq samples are “fuzzy” but nevertheless show strong correlations (R2=0.764-0.881) (FIG. 9A). The extent to which the same genes differ significantly between tumor normal in the two datasets was also determined. Using an FDR=0.05 cut-off for both FFPE-CUTAC and RNA-seq, it was found that 80-82% of genes were found in both lists: 52 of 63 for Yap1-driven tumors versus naïve brains, 268 of 336 for Pdgfb-driven versus naïve and 1519 of 1896 for RelA-driven versus naïve (Table 1). However, there is a striking difference in the specificity with which these genes are identified as illustrated by comparison of volcano plot displays: FFPE-CUTAC provides high specificity, where significant differences between cCREs are found for up to only ~0.5% of the >343,731 cCREs, almost exclusively at the upregulated corner of the volcano plots (high positive logz fold-change, high log 10 FDR) (FIGS. 5A-5D). In contrast, ~⅓ to ½ of 23,551 genes show significant differences between these tumorous and naïve brains using RNA-seq with massive, mostly symmetrical “volcanic eruptions” (FIG. 11).

Among the ten genes with the largest and most significant differences based on both FFPE-CUTAC and RNA-seq, most are concordant between FFPE-CUTAC and RNA-seq (Pdgfrb, Pdgfra, Col7a, A93000301Rik, Nkd2, Igf2, Tmem 181b-ps) (FIG. 7B). As expected, the FFPE-CUTAC profiles are enriched primarily at 5′ ends and RNA-seq at 3′ ends. However, three genes (Colla1, Colla2 and Dynlt1b) show striking discrepancies. For Dynlt1b, FFPE-CUTAC shows high promoter peaks for RelA-driven tumors and naïve brain not seen in Pdgfb- and Yap1-driven tumors, whereas RNA-seq shows nearly the opposite, which might be an example of regulatory elements becoming accessible because of repressor binding. Whatever the basis for such gene-specific discrepancies between the two profiling modalities, the fact that the concordant and discrepant genes showed up at or near the top of both gene lists, strongly suggests their relevance to the cancer phenotype. In conclusion there is overall excellent agreement between our FFPE-CUTAC data and previously published high-quality RNA-seq datasets. The very high specificity of FFPE-CUTAC data, together with its simple implementation and potential for automation, make it a unique and potentially useful modality for research and clinical applications.

### DISCUSSION

Fixation-related DNA and chromatin damage has thus far impeded the practical application of chromatin profiling to FFPEs (4). Here we have shown that improvements to the single-tube CUT&Tag-direct protocol to make it suitable for whole cells, together with heat-treatment of deparaffinized needle-extracted 10-micron FFPE sections, provides high-quality CUTAC data. By using an RNAPII-Ser5p antibody for paused RNAPII, our FFPE-CUTAC data provides a ground-truth interpretation of accessibility, applicable to both promoters and enhancers (28). While RNA-seq has been the go-to method for profiling the transcriptome, it only captures processed transcripts and as a result, routinely reports on a few thousands of abundant transcripts from a tissue. In contrast, the >300,000 genomic sites annotated as candidate cis-regulatory elements in the mouse genome can potentially provide direct information on transcriptional regulatory networks. Indeed, it was found that FFPE-CUTAC using an RNAPII-Ser5p identified >3000 candidate cis-regulatory elements at an FDR=0.05 that differ between tumorous and normal brains, and >1000 that differ between two different tumors. Therefore, it is anticipated that the improved CUT&Tag protocol will be widely adopted for FFPEs both using single-tube format and full automation for diagnosis, biomarker discovery and retrospective studies (20).

Cross-links and adducts resulting from the long incubations in formaldehyde necessary for long-term preservation cause DNA breaks and lesions that are serious impediments for most genomic methods applied to FFPEs. Indeed, standard CUT&Tag failed for the group that developed FACT-seq (16), and usable profiles for repressive H3K27me3 and H3K9me3 and gene-body H3K36me3 histone epitopes were not obtained. This may be attributed to the tight wrapping of DNA to lysine-rich histones, which are the most susceptible to cross-linking and formation of DNA adducts that result in DNA breaks during high-temperature cross-linking reversal (19). In contrast, nucleosome-depleted regions (NDRs) that are mapped using accessibility methods such as ATAC-seq and CUTAC are much better suited for FFPEs, as the protein machineries that occupy these sites are not especially lysine-rich. In particular, the YSPTSPS heptamer present in 52 tandem copies on the C-terminal domain of the largest subunit of RNAPII presents abundant lysine-free epitopes for CUT&Tag, and the use of low-salt tagmentation after stringent washes allows for tight binding of the Tn5 transposome within the confines of the NDR. It was previously shown that low-salt tagmentation allows for epitopes flanking promoters and enhancers, such as H3K4 methylations (17) and RNAPII epitopes (41), release subnucleosomal fragments preferentially, where tagmentation occurs within gaps in the chromatin landscape where these epitopes are located. FACT-seq improves yield with in vitro transcription from a T7 promoter inserted at single sites, however this strategy foregoes the advantage of the small size of NDRs at promoters and enhancers where nevertheless two Tn5s can fit with enough DNA in between to sequence. We might attribute the much better data quality that was obtained using CUTAC relative to FACT-seq to the very low probability of Tn5s inserting close to one another by random chance. Curiously, H3K27ac FFPE-CUTAC detected cCREs even more sensitively than standard H3K27ac CUT&RUN on frozen tissue, which might indicate that better reversal of cross-links at NDRs than at nucleosomes improves tagmentation within NDRs while nucleosomes remain relatively intractable. Indeed, by avoiding the use of degradative enzymes and using only heat to expose epitopes in a suitable buffer, it was found that bead-bound tissue shards from needle-extracted FFPEs are much easier to handle without damage than cells or nuclei, where lysis and sticking is a constant concern.

It was also discovered that DNA from Rhodococcus erythropolis, a species of bacteria that can live on paraffin wax as its only carbon source, is abundant, together with other bacteria, in the FFPE samples that were processed. As a result, lowering the amount of tissue in a paraffin slice results in a proportional increase in bacterial contamination. While this means that our protocol requires higher cell numbers than were required for FACT-seq, which began with enzymatic nuclei isolation (16), the bacterial contamination provides a convenient calibration standard in lieu of a spike-in. Paraffin-resident DNA has the unique advantage over spike-in strategies of being present in the sample before it is processed and as a result near-perfect anti-correlations are seen with cellular DNA as they compete with one another during PCR. In the present case, resident Rhodococcus DNA was utilized as a size standard, allowing the conclusion that the larger size distribution of tumor relative to naïve fragments has a biological basis, as the size differential was seen for both mouse nuclear and mitochondrial DNA but not for Rhodococcus DNA from the same samples.

In conclusion, paused chromatin profiling was shown to be conveniently and inexpensively performed on FFPEs in single PCR tubes. Only heat in a suitable buffer was utilized to reverse the cross-links while making the tissue sufficiently permeable, followed by needle extraction and a modified version of the CUT&Tag-direct protocol, which is routinely performed in many laboratories (18, 42). Data quality using low-salt tagmentation for antibody-tethered paused RNAPII chromatin accessibility mapping was found sufficient to distinguish cancer from normal tissues and resolve closely similar brain tumors. Using elevated levels of paused RNAPII as a discriminator, our study identified many known cancer-associated genes to be upregulated in tumors when compared to naïve brain, validating our approach.

### Methods

### Cell Lines

### FFPEs

Mice were euthanized and their brains removed and fixed at least 48 hours in neutral buffered formalin. Brains were sliced into five pieces and processed overnight in a tissue processor, mounted in a paraffin block and 10 micron sections were placed on slides. Slides were stored for varying times between 1 month to ~2 years before being deparaffinized and processed for FFPE-CUTAC. Deparaffinization was performed in Coplin jars using 2-3 changes of histology grade xylene over a 20 minute period, followed by 3-5 minute rinses in a 50:50 mixture of xylene: 100% ethanol, 100% ethanol (twice), 95% ethanol, 70% ethanol and 50% ethanol, then rinsed in deionized water. Slides were stored in distilled deionized water containing 0.02% sodium azide for up to 2 weeks before use.

### CUT&Tag-Direct for Whole Cells

Concanavalin A (ConA) coated magnetic beads (Bangs Laboratories, ca. no. BP531) were activated just before use with Ca⁺⁺ and Mn⁺⁺ as described (18). Frozen whole-cell aliquots were thawed at room temperature, split into PCR tubes and 5 μL ConA beads were added with gentle vortexing. All subsequent steps through to library preparation and purification followed the standard CUT&Tag-direct protocol (18), except that 1) all buffers from antibody incubation through tagmentation included 0.05% Triton®-X100; 2) the fragment release step was performed in 5 μl 1% SDS supplemented with 1:10 thermolabile proteinase K (New England Biolabs cat. no. P8111S) at 37° C. 1 hr followed by 58° C. 1 hr; 3) SDS was quenched by addition of 15 μl 6% Triton®-X100. A detailed step-by-step protocol is described in the example.

### FFPE-CUTAC

Tissue sections on deparaffinized slides were diced using a razor and scraped into a 1.7 mL low-bind tube containing 400 μl 800 mM Tris-HCl pH8.0, 0.05% Triton®-X100. Incubations were performed at 80-90° C. for 8-16 hours or as otherwise indicated either in a heating block or divided into 0.5 mL PCR tubes after needle extraction. Needle extraction was performed either before or after Concanavalin A-bead addition using a 1 ml syringe fitted with a 1″ 20 gauge needle with 20 up-and-down cycles, and in some cases was followed by 10 cycles with a ⅜″ 26 gauge needle. Other steps through to library preparation and purification followed the standard CUT&Tag-direct protocol (18) with the following exceptions: 1) all buffers from antibody incubation through tagmentation included 0.05% Triton®-X100; 2) the fragment release step was performed in 5 μl 1% SDS supplemented with 1:10 thermolabile proteinase K (New England Biolabs cat. no. P8111S) at 37° C. 1 hr followed by 58° C. 1 hr; 3) SDS was quenched by addition of 15 μl 6% Triton®-X100; 4) PCR was performed with an extension step (10 sec 98° C. denaturation, 30 sec 63° C. annealing and 1 min 72° C. extension for 12 cycles). A detailed step-by-step protocol is described in the example.

### DNA Sequencing and Data Processing

Libraries were sequenced on an Illumina HiSeq instrument with paired end 50×50 reads. Adapters were clipped by cutadapt (dx.doi.org/10.14806/ej.17.1.200) version 2.9 with parameters:

-j 8 -- nextseq-trim 20 -m 20 -a
(SEQ ID NO: 1)
AGATCGGAAGAGCACACGTCTGAACTCCAGTCA-A
(SEQ ID NO: 2)
AGATCGGAAGAGCGTCGTGTAGGGAAAGAGTGT-Z.

Clipped reads were aligned by Bowtie2 (43) to the UCSC Drosophila melanogaster Dm6 reference sequence (44) with parameters:

--very-sensitive-local--soft-clipped-unmapped-tlen--dovetail--no-mixed--no-discordant-q--phred33-1 10-X 1000.

Clipped reads were also aligned by Bowtie2 (43) to the UCSC Homo sapiens HG19 reference sequence (44) with parameters:--end-to-end--very-sensitive--no-overlap--no-dovetail--no-mixed--no-discordant-q--phred33-1 10-X 1000. Properly paired reads were extracted from the alignments by samtools (Version 1.9) (45). Normalized count tracks in bigwig format were made by bedtools (46) 2.30.0 genomecov which are the fraction of counts at each base pair scaled by the size of the reference sequence so that if the scaled counts were uniformly distributed there would be 1 at each position.

### Data Analysis

Differential analyses of FFPE-CUTAC and RNA-seq data were performed using the Voom/Limma option (47) on the Degust server (degust.erc.monash.edu/).

TABLE 1
Significant differences between FFPE-CUTAC datasets.

| | a) RNAPII-S5p counts¹ | | | | |
|---|---|---|---|---|---|
| Yap1 vs. Naïve | 808 | 5,788 | 5,784 | 4 | 99.9 |
| Pdgfb vs. Naïve | 1,576 | 19,172 | 19,163 | 9 | 99.9 |
| RelA vs. Naïve | 175 | 831 | 804 | 27 | 97 |
| Pdgfb vs. Yap1 | 11 | 27 | 27 | 0 | 100 |
| Yap1 vs. RelA | 15 | 102 | 90 | 12 | 88 |
| RelA vs. Pdgfb | 4 | 5,994 | 5,917 | 77 | 99 |
| Naïve vs. Naïve | 2 | 42 | 35 | 7 | 83 |
| Yap1 vs. Yap1 | 2 | 10 | 9 | 1 | 90 |
| | b) RNAPII-S5p summits² | | | | |
| Yap1 vs. Naïve | 12 | 293 | 293 | 0 | 100 |
| Pdgfb vs. Naïve | 21 | 345 | 345 | 0 | 100 |
| RelA vs. Naïve | 34 | 76 | 74 | 2 | 97 |
| RelA vs. Yap1 | 0 | 11 | 9 | 2 | — |
| Pdgfb vs. Yap1 | 1 | 4 | 4 | 0 | — |
| RelA vs. Pdgfb | 0 | 0 | 0 | 0 | — |
| | c) RNAPII-S2,5p summits² | | | | |
| Yap1 vs. Naïve | 213 | 1,369 | 1,152 | 217 | 84 |
| Pdgfb vs. Naïve | 91 | 1,497 | 1,488 | 9 | 99 |
| RelA vs. Naïve | 2926 | 7,263 | 6,694 | 569 | 92 |
| RelA vs. Yap1 | 135 | 540 | 427 | 113 | 79 |
| Pdgfb vs. Yap1 | 0 | 0 | 0 | 0 | — |
| RelA vs. Pdgfb | 6 | 10 | 6 | 4 | — |
| | d) RNAPII-S5p + S2,5p summits² | | | | |
| Yap1 vs. Naïve | 22 | 10,702 | 10,702 | 0 | 100 |
| Pdgfb vs. Naïve | 3,722 | 25,727 | 25,710 | 17 | >99.9 |
| RelA vs. Naïve | 4,709 | 23,084 | 23,005 | 79 | 99.7 |
| RelA vs. Yap1 | 0 | 0 | 0 | 0 | — |
| Pdgfb vs. Yap1 | 0 | 0 | 0 | 0 | — |
| RelA vs. Pdgfb | 0 | 0 | 0 | 0 | — |
| | H3K27ac summits² | | | | |
| Yap1vs. Naïve | 107,914 | 191,590 | 191,576 | 14 | >99.9 |
| Pdgfb vs. Naïve | 24,021 | 149,252 | 149,246 | 6 | >99.9 |
| RelA vs. Naïve | 7,219 | 47,598 | 47,573 | 25 | 99.9 |
| RelA vs. Yap1 | 1 | 3 | 0 | 3 | — |
| Pdgfb vs. Yap1 | 0 | 0 | 0 | 0 | — |
| RelA vs. Pdgfb | 1 | 1 | 0 | 1 | — |

¹FDR based on sum of normalized counts in each cCRE.
²FDR based on sum of normalized counts in summit within each cCRE as defined by the area under the highest value within a contiguous run of basepairs.

### CUTACs-FFPE v1

### Materials

Chilling device (e.g., metal heat blocks on ice or cold packs in an ice cooler) Pipettors (e.g., Rainin Classic Pipette 1 mL, 200 μL, 20 μL, and 10 μL) Disposable tips (e.g., Rainin 1 mL, 200 μL, 20 μL).
Disposable centrifuge tubes for reagents (15 mL or 50 mL) Standard 1.5 mL and 2 mL microfuge tubes
0.5 ml maximum recovery PCR tubes (e.g., Fisher cat. no. 14-222-294).
10 micron section from a formaldehyde-fixed paraffin-embedded tissue block affixed to a glass slide.
Concanavalin A (ConA)-coated magnetic beads (Bangs Laboratories, ca. no. BP531) Strong magnet stand (e.g., Miltenyi Macsimag separator, cat. no. 130-092-168) Vortex mixer (e.g., VWR Vortex Genie)
Mini-centrifuge (e.g., VWR Model V) Tube Rotator or Nutator
PCR thermocycler (e.g., BioRad/MJ PTC-200)
1 ml syringe+1″ 22 gauge and 3/16″ 26 gauge needles Xylenes (Histology grade)
Mineral Oil (Sigma cat. no 330779) Ethanol (Decon Labs, cat. no. 2716)
Distilled, deionized or RNAse-free H2O (dH₂O e.g., Promega, cat. no. P1197).
1 M Hydroxyethyl piperazineethanesulfonic acid pH 7.9 (HEPES (K+); Sigma-Aldrich, cat. no. H3375)
1 M Manganese Chloride (MnCl2; Sigma-Aldrich, cat. no. 203734) 1 M Calcium Chloride (CaCl₂); Fisher, cat. no. BP510)
1 M Potassium Chloride (KCl; Sigma-Aldrich, cat. no. P3911)
Roche Complete Protease Inhibitor EDTA-Free tablets (Sigma-Aldrich, cat. no. 5056489001).
1 M Hydroxyethyl piperazineethanesulfonic acid pH 7.5 (HEPES (Na+); Sigma-Aldrich, cat. no. H3375)
5 M Sodium chloride (NaCl; Sigma-Aldrich, cat. no. S5150-1L) 2 M Spermidine (Sigma-Aldrich, cat. no. S0266)
10% Triton® X-100 (Sigma-Aldrich, cat. no. X100).
0.5 M Ethylenediaminetetraacetic acid (EDTA; Research Organics, cat. no. 3002E) 200X Bovine Serum Albumen (BSA, NEB, cat no. B9001S).
Antibody to an epitope of interest. Because in situ binding conditions are more like those for immunofluorescence (IF) than those for ChIP, it is suggested to choose IF-tested antibodies if CUT&RUN/Tag-tested antibodies are not available.
CUTAC control antibody to RNA Polymerase II Phospho-Rpb1 CTD Serine-5 phosphate (PolIIS5P, CST #13523 (D9N5I)).
Secondary antibody, e.g., guinea pig a-rabbit antibody (Antibodies online cat. no. ABIN101961) or rabbit a-mouse antibody (Abcam cat. no. ab46540).
Protein A/G-Tn5 (pAG-Tn5) fusion protein loaded with double-stranded adapters with 19mer Tn5 mosaic ends Epicypher cat. no. 15-1117
Thermolabile Proteinase K (NEB P8111S)
1 M Magnesium Chloride (MgCl2; Sigma-Aldrich, cat. no. M8266-100G)
1 M [tris(hydroxymethyl)methylamino] propanesulfonic acid (TAPS) pH 8.5 (with NaOH) 1,6-hexanediol (Sigma-Aldrich cat. no. 240117-50G).
N,N-dimethylformamide (Sigma-Aldrich cat. no. D-8654-250 mL) NEBNext 2X PCR Master mix (ME541L).
PCR primers: 10 μM stock solutions of i5 and i7 primers with unique barcodes [Buenrostro, J. D. et al. Nature 523:486 (2015)] in 10 mM Tris pH 8. Standard salt-free primers may be used. Nextera or NEBNext primers are not recommended.
10% Sodium dodecyl sulfate (SDS; Sigma-Aldrich, cat. no. L4509).
SPRI paramagnetic beads (e.g., HighPrep PCR Cleanup Magbio Genomics cat. no. AC-60500).
10 mM Tris-HCl pH 8.0

Note: Deparaffinization uses xylene, a toxic aromatic compound, and should be performed in a fume hood. There are no hazardous materials or dangerous equipment used in other steps of this protocol, however appropriate lab safety training is recommended. Reagent Setup for up to 16 samples:

1. Cross-link reversal buffer-Mix 800 μL Tris-HCl pH8.0, 195 μL dH2O and 5 μL Triton®-X100

Binding buffer-Mix 200 μl 1M HEPES-KOH PH 7.9*, 100 μl 1M KCl, 10 μl 1M CaCl₂ and 10 μl 1M MnCl₂, and bring the final volume to 10 mL with dH₂O. Store the buffer at 4° C. for up to several months. * HEPES-NaOH pH 7.5 is OK.

Triton®-Wash buffer Mix 1 mL 1 M HEPES pH 7.5, 1.5 mL 5 M NaCl, 250 μl Triton®-X100 and 12.5 μl 2 M spermidine, bring the final volume to 50 mL with dH2O, and add 1 Roche Complete Protease Inhibitor EDTA-Free tablet. Store the buffer at 4° C. for up to 2 days.

Antibody buffer Mix 5 μl 200X BSA with 1 ml Triton®-Wash buffer and chill on ice. CUTAC-DMF Tagmentation buffer Mix 780 μl dH20, 200 μl N,N-dimethylformamide, 10 μl 1 M TAPS pH 8.5, 5 μl Triton®-X100 and 5 μl 1 M MgCl2 (10 mM TAPS, 5 mM MgCl2, 20% DMF, 0.05% Triton®-X100). Store the buffer at 4° C. for up to 1 week.

TAPS wash buffer Mix 1 mL dH20, 10 μl 1 M TAPS pH 8.5, 0.4 μl 0.5 M EDTA (10 mM TAPS, 0.2 mM EDTA). Store at room temperature.

1% SDS/ProtK Release solution (For 32 samples) Mix 20 μl 10% SDS and 2 μl 1 M TAPS pH 8.5 in 158 μl dH2O. Just before use add 20 μL Thermolabile Proteinase K (NEB cat. no. P8111S).

6% Triton® Mix 600 μl 10% Triton®-X100+400 μl dH2O. Store at room temperature.

Option 1:1. Deparaffinize FFPE section affixed to slide using xylene (1 hr).

2. In a fume hood, immerse slide(s) in xylene for 10 min, then transfer to fresh xylene for 5 min.

Note: For a non-toxic deparaffinization/processing protocol, skip to Option 2: Deparaffinize FFPE section affixed to microscope slide with mineral oil.

3. Transfer slide(s) to a 50:50 mixture of xylene and 100% ethanol for 3 min. This can be reused or discarded in toxic waste container.

4. Transfer slide(s) to 100% ethanol for 3 min. Repeat once.

5. Immerse slide(s) 95% ethanol for 3 min.

6. Immerse slide(s) in 70% ethanol for 3 min.

7. Immerse slide(s) in 50% ethanol for 3 min.

8. Rinse slide(s) with tap water or tap-distilled water with change(s).

Process deparaffinized FFPE sample for CUT&Tag (1.5 hr).

9. Dice with a razor blade, scrape and pick up the 10 μm section and deposit it into a 1.5 mL tube containing 400 μL Cross-link reversal buffer (800 mM Tris-HCl pH8.0, 0.05% Triton®-X100).

Note: Deparaffinized slides or scrapes have been stored in 0.005% sodium azide+100 μg/ml ampicillin for up to a few weeks.

10. Incubate 8-16 hours at 85° C. in a heating block.

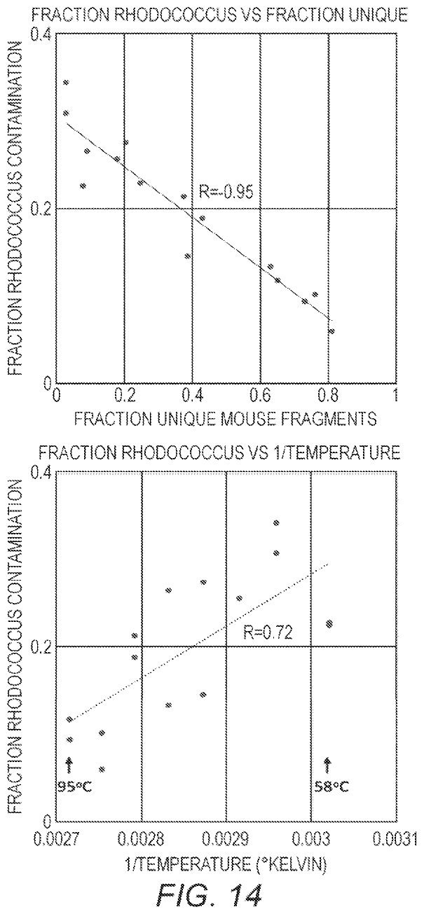

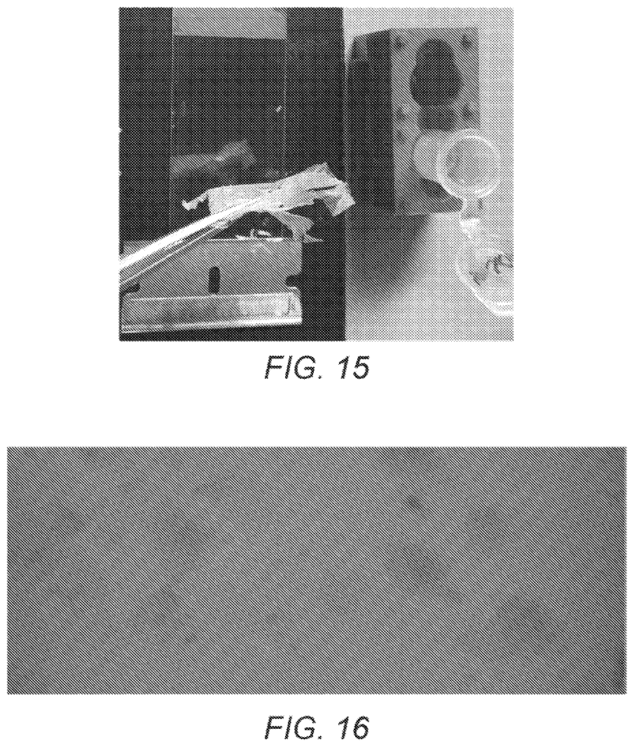

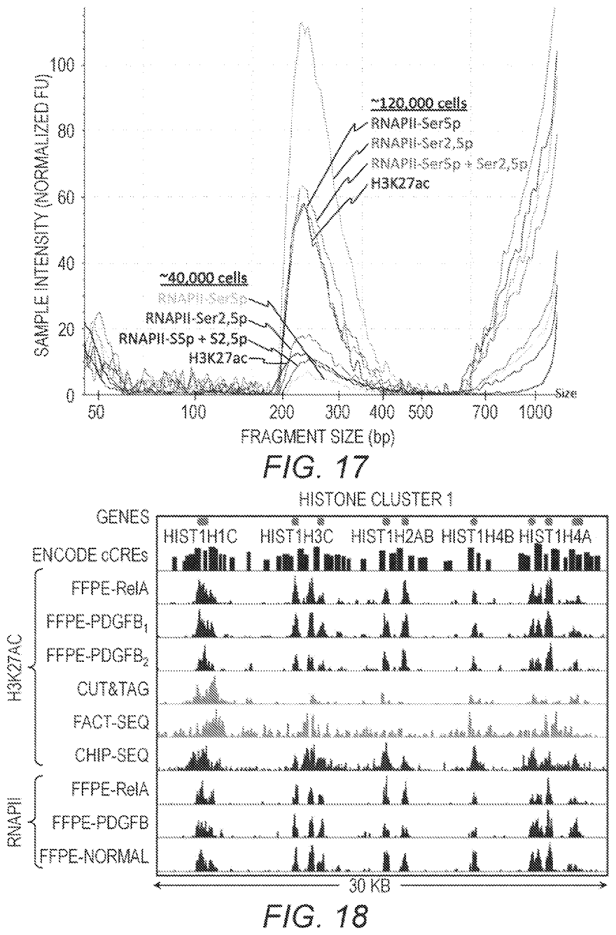

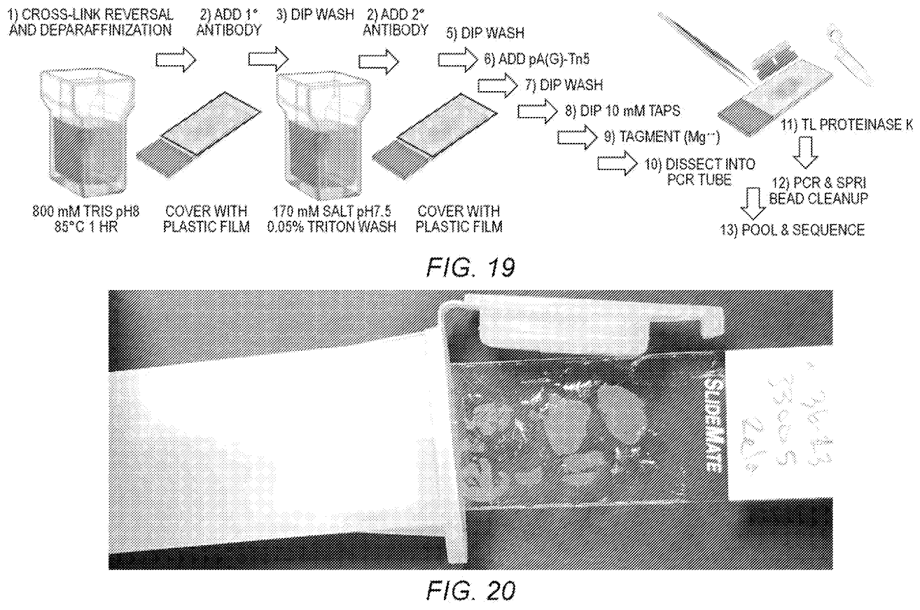

Note: The rate of formaldehyde cross-link reversal increases with temperature (Henikoff S, Henikoff J G, Kaya-Okur H S, Ahmad K. Efficient chromatin accessibility mapping in situ by nucleosome-tethered tagmentation. Elife. 2020 Nov. 16;9: e63274.), where 1 hr at 65° C. is calculated to be sufficient for near-complete reversal. For FFPEs, higher temperatures up to 90-95° C. were used, which denatures contaminating DNA (FIG. 14) so it is no longer a substrate for Tn5 but spares chromatin in situ. It was found that mappability improves with incubations at 80-90° C. overnight (8-16 hr).

11. Resuspend and withdraw enough of the ConA bead slurry, ensuring that there will be ~5 μl for each final sample. For example, 160 μl ConA bead slurry was added to 1.5 mL of Binding buffer for 32 samples.

Note: Prepare beads shortly before use. A 7.5 μL bead slurry per sample has been used with excellent results.

12. Mix by pipetting. Place the tube on a magnet stand to clear (~1 min).

13. Withdraw the supernatant completely, and remove the tube from the magnet stand. Add 2 mL Binding buffer (for 32 samples) and mix by vortexing.

14. Add 240 μl to each tube while vortexing, where one section will be used for 4 replicate samples (60 μL per sample). Place on rotator 10-20 min.

Note: For 10 micron sections, consistent results have been obtained with ¼th of a slide per sample. Excellent results have also been obtained with ⅛th of a 10 micron section. This protocol has also been applied to 5 micron sections.

15. Pass through a 22 gauge 1″ needle using a Luer-lock glass syringe 20 times to break up tissue.

Note: Use firm plunges but not so hard as to cause overflowing. This procedure may result in foaming. To clear the foam, spin 3000×g for 1 minute, then vortexing will disperse the small shards of 10 μm thick tissue.

Option 2: Deparaffinize FFPE section affixed to slide with mineral oil.

16. Scrape off excess paraffin and continuously scrape all of part of the paraffin-embedded 10-μm section. Using tweezers (e.g., #3 watchmaker's forceps) carefully lift off the curls and plunge into mineral oil in a 2 ml tube using 100 μl per final sample.

17. Heat 10 min 80° C. to dissolve the curls. Add 1 volume Cross-link reversal buffer (800 mM Tris-HCl pH8.0, 0.05% Triton®-X100. Vortex 10 sec, centrifuge briefly, and pass 20 times through a 22 gauge needle on a 1 ml Luer Lock syringe. Centrifuge at 3000×g 1 min. The paraffin will partially solidify in the upper layer while the tissue partitions to the lower (aqueous) layer.

18. Heat briefly to melt the upper layer and remove without disturbing the lower layer using a wide-bore or cut-off 200 μl low-bind pipette tip. Add 1 volume mineral oil, vortex, centrifuge, heat and decant the upper layer.

19. Repeat Step 18 until the interface is clear or nearly so. Using a wide-bore 200 μl pipette tip transfer 100 μl to PCR tubes.

Note: It is not necessary to remove the remaining thin layer of mineral oil, which could result in loss of tissue adhering to the meniscus. This small amount of oil is eventually lost during bead washes.

20. Place tubes in a thermocycler and incubate at 85° C. for at least 2 hours. It was found that mappability improves with incubations at 80-90° C. overnight (8-16 hr).

21. Resuspend and withdraw enough of the ConA bead slurry, ensuring that there will be ~5 μl for each final sample. For example, 160 μl ConA bead slurry were added to 1.5 mL of Binding buffer for 32 samples. Place the pipette tip below the meniscus to avoid coating the beads with oil and discharge the beads while mixing by pipetting.

22. Mix by pipetting. Place the tube on a magnet stand to clear (~1 min).

23. Withdraw the supernatant completely, and remove the tube from the magnet stand. Add 2 mL Binding buffer (for 32 samples) and mix by vortexing.

24. Resuspend in 160 μL Binding buffer. Add 5 μl to each sample while vortexing. Place on Rotator 10-20 min.

### Bind Primary Antibody (2 hr)

25. After a quick spin, place the tubes on the magnet stand to clear and withdraw the liquid.

Note: The protocol for FFPEs is similar to CUT&Tag-direct Version 3 and can be performed in parallel with native or lightly cross-linked nuclei or whole cells. Although whole cells are not appropriate with that version, including 0.05% Triton®-X100 from antibody binding to tagmentation stabilizes the bead pellet and permeabilizes cells such that by the time of tagmentation the remaining cellular material is no longer inhibitory for PCR. Now 0.05% Triton®-X100 is added by default for all CUT&Tag and CUTAC protocols, including for single cells. It was found that best results are obtained adding 1:10 thermolabile proteinase K to the fragment-release solution and incubating as in this protocol pre-PCR.

26. For each CUT&Tag and CUTAC sample, mix the primary antibody 1:25 with Antibody buffer. Resuspend beads in 25 μl per sample followed by vortexing.

Note: For FFPEs 1:25 antibody dilutions were used and incubated 1-2 hr to overnight at room temperature to maximize antibody penetration. For long RT incubations sodium azide was added to a final concentration of 0.005% as a precaution to prevent microbial growth. We have used Pol2Ser5 (CST (D9N51) mAb #13523), Pol2Ser2,5 (CST (D1G3K) mAb #13546), Pol2Ser5+Ser2,5 (mixed) and H3K27ac (Abcam ab4729) with success. Pol2Ser5 results in the sharpest peaks but typically with reduced yield relative to the other single antibodies.

27. Place on a rotator at room temperature and incubate at least 1 hr on rotator at room temperature.

Note: It was found that 1 hr incubations at room temperature suffice for primary and secondary antibodies and pAG-Tn5. For overnight RT incubations, add 0.005% sodium azide as a precaution to inhibit bacterial growth.

Bind secondary antibody

28. After a quick spin, place the tubes on the magnet stand to clear and withdraw the liquid.

29. Mix the secondary antibody 1:100 in Wash buffer and squirt in 25 μl per sample followed by vortexing.

Note: The secondary antibody step is required for CUT&Tag to increase the number of protein A binding sites for each bound antibody. It was found that without the secondary antibody, the efficiency is very low.

30. Place the tubes on a rotator or nutator and rotate or nutate at room temperature for 1 hr.

31. After a quick spin (<500×g or just enough to remove the liquid from the sides of the tube), place the tubes on the magnet stand to clear and remove and discard the supernatant with two successive draws, using a 20 μl tip with the pipettor set for maximum volume.

32. With the tubes still on the magnet stand, carefully add 500 μl of Wash buffer. The surface tension will cause the beads to slide up along the side of the tube closest to the magnet.

33. Slowly withdraw 460 μl of supernatant with a 1 mL pipette tip without disturbing the beads.

Note: To remove the supernatant, set the pipettor to 460 μl, and keep the plunger depressed while lowering the tip to the bottom. The liquid level will rise to near the top completing the wash. Then ease off on the plunger until the liquid is withdrawn and remove the pipettor. During liquid removal, the surface tension will drag the beads down the tube. A small drop of liquid that is left behind will be removed in the next step.

Note: Bead-bound shards from FFPEs stick to the sides of low-bind PCR tubes, which is especially conspicuous after Wash buffer removal and vortexing is not sufficient to wet them. Therefore, tubes should be mixed by inversion after vortexing.

34. After a quick spin (<500×g or just enough to remove the liquid from the sides of the tube), place the tubes back into the magnet stand and remove the remaining supernatant with a 20 μl pipettor multiple times if necessary, to remove the entire supernatant without disturbing the beads. Proceed immediately to the next step.

Bind pA-Tn5 Adapter Complex (1.5 hr)

35. Mix pAG-Tn5 pre-loaded adapter complex in Triton®-Wash buffer following the manufacturer's instructions (e.g., 1:20 for EpiCypher pAG-Tn5).

Note: This protocol is not recommended for “homemade” pA-Tn5 following our purification protocol, because the contaminating E. coli DNA will be preferentially tagmented relative to the less accessible FFPE DNA under the stringent 55° C. conditions used here. If homemade pA-Tn5 is used, it is important to minimize the amount added (≤1:200).

36. Pipette in 25 μl per sample of the pA-Tn5 mix followed by vortexing.

37. After a quick spin, place the tubes on a rotator at room temperature for 1 hr or 4° C. overnight.

38. After incubating in the rotator, perform a quick spin and place the tubes in the magnet stand.

39. Carefully remove the supernatant using a 20 μl pipettor as in Step 31.

40. With the tubes still on the magnet stand, add 500 μl of the Triton®-Wash buffer.

41. Slowly withdraw 460 μl with a 1 ml pipette tip without disturbing the beads as in Step 33.

42. After a quick spin, place the tubes back on the magnet stand and remove and discard the supernatant with a 20 μL pipettor using multiple draws. Proceed immediately to Step 43.

Tagment (1.5 hr, Performed in Parallel with Standard CUT&Tag and CUTAC

43. Resuspend the bead/FFPE pellet in 50 μl CUTAC-DMF tagmentation solution (5 mM MgCl₂, 10 mM TAPS, 20% DMF, 0.05% Triton®-X) while vortexing. Incubate 1 hr 55° C. in thermocycler.

Note: N,N-dimethylformamide is a dehydrating compound resulting in improved tethered Tn5 accessibility and library yield. Conditions used for FFPEs are the most stringent tested in Henikoff S, Henikoff J G, Kaya-Okur H S, Ahmad K. Efficient chromatin accessibility mapping in situ by nucleosome-tethered tagmentation. Elife. 2020 Nov. 16;9: e63274. doi: 10.7554/eLife.63274-FIG. 3-figure supplement 2.

44. Place tubes on a magnet stand and remove and discard the supernatant with a 20 μL pipettor using multiple draws then resuspend the beads in 50 μL TAPS wash and mix by vortexing.

### Fragment Release (2.5 hr)

45. After a quick spin, place tubes on the magnet stand, and withdraw the liquid with a 20 μL pipettor using multiple draws.

46. Resuspend the beads by squirting in 5 μL 1% SDS/ProtK Release solution followed by vortexing.

47. After a quick spin, incubate at 37° C. for 1 hr and 58° C. for 1 hr (programmed in succession in a PCR cycler with a heated lid) to release pA-Tn5 from the tagmented DNA.

PCR (1 hr)

48. To the PCR tube containing the bead slurry add 15 μl of Triton® neutralization solution+2 μl of 10 μM Universal or barcoded i5 primer+2 μl of 10 μM uniquely barcoded i7 primers, using a different barcode for each sample. Vortex on full speed and place tubes in the metal tube holder on ice.

Note: Indexed primers are described by Buenrostro, J. D. et al. Single-cell chromatin accessibility reveals principles of regulatory variation. Nature 523:486 (2015). Nextera or NEB primers are not generally recommended, which might not anneal efficiently using this PCR protocol.

49. Add 25 μl NEBnext (non-hot-start), vortex to mix, and perform a quick spin. Place the tubes immediately in the thermocycler and proceed immediately with the PCR.

50. Begin the cycling program with a heated lid on the thermocycler:
Cycle 1:58° C. for 5 min (gap filling)
Cycle 2:72° C. for 5 min (gap filling)
Cycle 3:98° C. for 5 min
Cycle 4:98° C. for 10 sec
Cycle 5:63° C. for 30 sec
Cycle 6:72° C. for 1 min
Repeat Cycles 4-6 11 times
Hold at 8° C.

Note: CUT&Tag uses short 2-step 10 sec cycles to favor amplification of nucleosomal and smaller fragments. However, after cross-link reversal, DNA in FFPEs are small and PCR amplicon sizes <120 bp are recommended (Do and Dobrovic, Clin. Chem. 61 (1): 64-71 (2015)), which obviates the need to minimize the contribution of large DNA fragments. Insertion of a 1 min 72° C. extension and lengthening of the 63° C. annealing time from 10 sec to 30 sec results in better read-through of damaged DNA by Taq polymerase, resulting in a higher fraction of mappable reads than using the 2-step cycle favored for CUT&Tag and CUTAC.

Note: No more than 12 cycles are recommended. Do not add extra PCR cycles to see a signal by capillary gel electrophoresis (e.g., Tapestation). Extra PCR cycles reduce the complexity of the library and may favor contaminating bacterial DNA from the paraffin (FIG. 14).

Post-PCR Clean-up (30 min)
51. After the PCR program ends, remove tubes from the thermocycler and add 65 μL of SPRI beads (ratio of 1.3 μL of SPRI beads to 1 μL of PCR product). Mix by pipetting up and down.
52. Let sit at room temperature 5-10 min.
53. Place on the magnet stand for a few minutes to allow the solution to clear.
54. Remove and discard the supernatant.
55. Keeping the tubes in the magnet stand, add 200 μL of 80% ethanol.
56. Completely remove and discard the supernatant.
57. Repeat Steps 55 and 56.
58. Perform a quick spin and remove the remaining supernatant with a 20 μl pipette, avoiding air drying the beads by proceeding immediately to the next step.
59. Remove from the magnet stand, add 22 μl 10 mM Tris-HCl pH 8 and vortex at full speed. Let sit for 5 min to 1 hr.
60. Place on the magnet stand and allow to clear.
61. Remove the liquid to a fresh 1.5 mL tube with a pipette, avoiding transfer of beads.

### Tapestation Analysis and DNA Sequencing

62. Determine the size distribution and concentration of libraries by capillary electrophoresis using an Agilent 4200 TapeStation with D1000 reagents or equivalent.

Note: Quantification by Tapestation was used to estimate library concentration and dilute each library to 2 nM before pooling based on fragment molarity in the 175-500 bp range. The concentration 2 nM has been determined empirically as the optimal library concentration used in the HiSeq by the Fred Hutch Genomics Shared Resource.

63. Mix barcoded libraries to achieve equal representation as desired aiming for a final concentration as recommended by the manufacturer. After mixing, perform an SPRI bead cleanup if needed to remove any residual PCR primers.

64. Perform paired-end Illumina sequencing on the barcoded libraries following the manufacturer's instructions.

Note: We currently use paired-end 50×50 sequencing on an Illumina Next-Seq, obtaining ~400 million total mapped reads, or ~4 million per sample when there are 96 samples mixed to obtain approximately equal molarity.

### Data Processing and Analysis

65. Align paired-end reads to hg19 using Bowtie2 version 2.3.4.3 with options:--end-to-end--very-sensitive--no-unal--no-mixed--no-discordant--phred33-I 10-X 700. For mapping E. coli carry-over fragments, we also use the—no-overlap—no-dovetail options to avoid possible cross-mapping of the experimental genome to that of the carry-over E. coli DNA that is used for calibration. Tracks are made as bedgraph files of normalized counts, which are the fraction of total counts at each basepair scaled by the size of the hg19 genome.

Note: To calibrate samples in a series for samples done in parallel using the same antibody counts of E. coli fragments carried over with the pA-Tn5 were used for an ordinary spike-in. Our sample script in Github can be used to calibrate based on either a spike-in or E. coli carry-over DNA.

66. The CUT&Tag Data Processing and Analysis Tutorial on Protocols.io, available at doi: 10.17504/protocols.io.bjk2kkye, provides step-by-step guidance for mapping and analysis of CUT&Tag sequencing data. Most data analysis tools used for ChIP-seq data, such as bedtools, Picard and deepTools, can be used on CUT&Tag data. Analysis tools designed specifically for CUT&RUN/Tag data include the SEACR peak caller also available as a public web server at fredhutch.org and CUT&RUNTools, available at Zhu, Q., Liu, N., Orkin, S. H. et al. CUT&RUNTools: a flexible pipeline for CUT&RUN processing and footprint analysis. Genome Biol 20, 192 (2019); doi: 10.1186/s13059-019-1802-4.

### REFERENCES

1. Saqi A. The State of Cell Blocks and Ancillary Testing: Past, Present, and Future. Arch Pathol Lab Med. 2016; 140 (12): 1318-22.
2. Blow N. Tissue preparation: Tissue issues. Nature. 2007; 448 (7156): 959-63.
3. Armstrong SA, Henikoff S, Vakoc CR. Chromatin Deregulation in Cancer. Cold Spring Harbor, New York: Cold Spring Harbor Press; 2017.
4. Amatori S, Fanelli M. The Current State of Chromatin Immunoprecipitation (ChIP) from FFPE Tissues. International journal of molecular sciences. 2022; 23 (3).
5. Kaneko S, Mitsuyama T, Shiraishi K, Ikawa N, Shozu K, Dozen A, et al. Genome-Wide Chromatin Analysis of FFPE Tissues Using a Dual-Arm Robot with Clinical Potential. Cancers. 2021; 13 (9).
6 Font-Tello A, Kesten N, Xie Y, Taing L, Vareslija D, Young L S, et al. FiTAc-seq: fixed-tissue ChIP-seq for H3K27ac profiling and super-enhancer analysis of FFPE tissues. Nat Protoc. 2020; 15 (8): 2503-18.
7 Zhong J, Ye Z, Clark CR, Lenz SW, Nguyen JH, Yan H, et al. Enhanced and controlled chromatin extraction from FFPE tissues and the application to ChIP-seq. BMC Genomics. 2019; 20 (1): 249.
8. Amatori S, Persico G, Paolicelli C, Hillje R, Sahnane N, Corini F, et al. Epigenomic profiling of archived FFPE tissues by enhanced PAT-ChIP (EPAT-ChIP) technology. Clinical epigenetics. 2018; 10 (1): 143.
9. Fanelli M, Amatori S, Barozzi I, Soncini M, Dal Zuffo R, Bucci G, et al. Pathology tissue-chromatin immunoprecipitation, coupled with high-throughput sequencing, allows the epigenetic profiling of patient samples. Proc Natl Acad Sci USA. 2010; 107 (50): 21535-40.
10. Cejas P, Li L, O'Neill NK, Duarte M, Rao P, Bowden M, et al. Chromatin immunoprecipitation from fixed clinical tissues reveals tumor-specific enhancer profiles. Nat Med. 2016; 22 (6): 685-91.
11. Buenrostro JD, Giresi PG, Zaba LC, Chang HY, Greenleaf WJ. Transposition of native chromatin for fast and sensitive epigenomic profiling of open chromatin, DNA-binding proteins and nucleosome position. Nat Methods. 2013; 10 (12): 1213-8.
12. Skene PJ, Henikoff S. An efficient targeted nuclease strategy for high-resolution mapping of DNA binding sites. eLife. 2017; 6: e21856.
13. Kaya-Okur H S, Wu SJ, Codomo CA, Pledger ES, Bryson TD, Henikoff J G, et al. CUT&Tag for efficient epigenomic profiling of small samples and single cells. Nat Commun. 2019; 10:1930.
14. Yadav RP, Polavarapu VK, Xing P, Chen X. FFPE-ATAC: A Highly Sensitive Method for Profiling Chromatin Accessibility in Formalin-Fixed Paraffin-Embedded Samples. Current protocols. 2022; 2 (8): e535.
15. Zhang H, Polavarapu VK, Xing P, Zhao M, Mathot L, Zhao L, et al. Profiling chromatin accessibility in formalin-fixed paraffin-embedded samples. Genome Res. 2022; 32 (1): 150-61.
16. Zhao L, Xing P, Polavarapu VK, Zhao M, Valero-Martinez B, Dang Y, et al. FACT-seq: profiling histone modifications in formalin-fixed paraffin-embedded samples with low cell numbers. Nucleic Acids Res. 2021; 49 (21): e125.
17. Henikoff S, Henikoff J G, Kaya-Okur H S, Ahmad K. Efficient chromatin accessibility mapping in situ by nucleosome-tethered tagmentation. eLife. 2020; 9: e63274.
18. Henikoff S, Henikoff J G, Ahmad K. Simplified Epigenome Profiling Using Antibody-tethered Tagmentation. bio-protocol. 2021; 11 (11): e4043.
19. Oba U, Kohashi K, Sangatsuda Y, Oda Y, Sonoda KH, Ohga S, et al. An efficient procedure for the recovery of DNA from formalin-fixed paraffin-embedded tissue sections. Biology methods & protocols. 2022; 7 (1): bpac014.
20. Janssens D H, Meers MP, Wu SJ, Babaeva E, Meshinchi S, Sarthy μF, et al. Automated CUT&Tag profiling of chromatin heterogeneity in mixed-lineage leukemia. Nat Genet. 2021; 53 (11): 1586-96.
21. Kennedy-Darling J, Smith LM. Measuring the formaldehyde Protein-DNA cross-link reversal rate. Anal Chem. 2014; 86 (12): 5678-81.
22. Robbe P, Popitsch N, Knight SJL, Antoniou P, Becq J, He M, et al. Clinical whole-genome sequencing from routine formalin-fixed, paraffin-embedded specimens: pilot study for the 100,000 Genomes Project. Genetics in medicine: official journal of the American College of Medical Genetics. 2018; 20 (10): 1196-205.
23. Leers MP, Schutte B, Theunissen PH, Ramaekers FC, Nap M. Heat pretreatment increases resolution in DNA flow cytometry of paraffin-embedded tumor tissue. Cytometry. 1999;35 (3): 260-6.
24. Rodig SJ. Preparing Paraffin Tissue Sections for Staining. Cold Spring Harbor protocols. 2021; 2021 (3).
25. Ivshina IB, Krivoruchko AV, Kuyukina MS, Peshkur TA, Cunningham CJ. Adhesion of Rhodococcus bacteria to solid hydrocarbons and enhanced biodegradation of these compounds. Sci Rep. 2022; 12 (1): 21559.
26. Rodrigues CJC, de Carvalho C. Phenotypic Adaptations Help Rhodococcus erythropolis Cells during the Degradation of Paraffin Wax. Biotechnology journal. 2019; 14 (8): e1800598.
27. Brahma S, Henikoff S. RNA Polymerase II, the BAF remodeler and transcription factors synergize to evict nucleosomes. biorxiv. 2023; doi: 10.1101/2023.01.22.525083.
28. Andersson R, Sandelin A, Danko CG. A unified architecture of transcriptional regulatory elements. Trends Genet. 2015; 31 (8): 426-33.
29. Li M, Hou Y, Zhang Z, Zhang B, Huang T, Sun A, et al. Structure, activity and function of the lysine methyltransferase SETD5. Frontiers in endocrinology. 2023; 14:1089527.
30. Chen Y, Cen L, Guo R, Huang S, Chen D. Roles and mechanisms of phosphoglycerate kinase 1 in cancer. Bull Cancer. 2022; 109 (12): 1298-307.
31. Liu S, Chen L, Zeng J, Chen Y. A prognostic model based on the COL1A1-network in gastric cancer. American journal of translational research. 2023; 15 (3): 1640-53.
32. Belfiore A, Rapicavoli RV, Le Moli R, Lappano R, Morrione A, De Francesco E M, et al. IGF2: A Role in Metastasis and Tumor Evasion from Immune Surveillance? Biomedicines. 2023; 11 (1).
33. Masuzaki R, Kanda T, Sasaki R, Matsumoto N, Nirei K, Ogawa M, et al. Suppressors of Cytokine Signaling and Hepatocellular Carcinoma. Cancers. 2022; 14 (10).
34 Farzaneh M, Masoodi T, Ghaedrahmati F, Radoszkiewicz K, Anbiyaiee A, Sheykhi-Sabzehpoush M, et al. An updated review of contribution of long noncoding RNA-NEAT1 to the progression of human cancers. Pathol Res Pract. 2023; 245:154380.
35. Yan H, Jiang F, Yang J. Association of beta-Catenin, APC, SMAD3/4, Tp53, and Cyclin D1 Genes in Colorectal Cancer: A Systematic Review and Meta-Analysis. Genetics research. 2022; 2022:5338956.
36. Renfro Z, White BE, Stephens KE. CCAAT enhancer binding protein gamma (C/EBP-gamma): An understudied transcription factor. Advances in biological regulation. 2022; 84:100861.
37. Zhao F, Li C, Wu Y, Xia J, Zeng M, Li T, et al. Connective Tissue Growth Factor in Digestive System Cancers: A Review and Meta-Analysis. BioMed research international. 2020; 2020:8489093.
38. Ozawa T, Arora S, Szulzewsky F, Juric-Sekhar G, Miyajima Y, Bolouri H, et al. A De Novo Mouse Model of C11orf95-RELA Fusion-Driven Ependymoma Identifies Driver Functions in Addition to NF-kappaB. Cell Rep. 2018; 23 (13): 3787-97.
39. Szulzewsky F, Arora S, Arakaki AKS, Sievers P, Almiron Bonnin DA, Paddison P J, et al. Both YAP1-MAML2 and constitutively active YAPI drive the formation of tumors that resemble NF2 mutant meningiomas in mice. Genes Dev. 2022; 36 (13-14): 857-70.
40. Szulzewsky F, Arora S, Hoellerbauer P, King C, Nathan E, Chan M, et al. Comparison of tumor-associated YAP1 fusions identifies a recurrent set of functions critical for oncogenesis. Genes Dev. 2020; 34 (15-16): 1051-64.
41. Janssens D H, Otto DJ, Meers MP, Setty M, Ahmad K, Henikoff S. CUT&Tag2 for1: a modified method for simultaneous profiling of the accessible and silenced regulome in single cells. Genome Biol. 2022; 23 (1): 81.
42. Henikoff S, Ahmad K. In situ tools for chromatin structural epigenomics. Protein Sci. 2022; 31 (11): e4458.
43. Langmead B, Salzberg SL. Fast gapped-read alignment with Bowtie 2. Nat Methods. 2012; 9 (4): 357-9.
44. Nassar LR, Barber GP, Benet-Pages A, Casper J, Clawson H, Diekhans M, et al. The UCSC Genome Browser database: 2023 update. Nucleic Acids Res. 2022.
45. Danecek P, Bonfield JK, Liddle J, Marshall J, Ohan V, Pollard M O, et al. Twelve years of SAMtools and BCFtools. GigaScience. 2021; 10 (2).
46. Quinlan AR. BEDTools: The Swiss-Army Tool for Genome Feature Analysis. Current protocols in bioinformatics. 2014; 47:11 2 1-34.
47. Law CW, Chen Y, Shi W, Smyth GK. voom: Precision weights unlock linear model analysis tools for RNA-seq read counts. Genome Biol. 2014; 15 (2): R29.
48. Kaya-Okur H S, Janssens D H, Henikoff J G, Ahmad K, Henikoff S. Efficient low-cost chromatin profiling with CUT&Tag. Nat Protoc. 2020; 15 (10): 3264-83.

### Example 2. CUTAC for FFPEs V.2

### Materials:

Chilling device (e.g., metal heat blocks on ice or cold packs in an ice cooler)
Pipettors (e.g., Rainin Classic Pipette 1 mL, 200 μL, 20 μL, and 10 μL)
Disposable tips (e.g., Rainin 1 mL, 200 μL, 20 μL)
Disposable centrifuge tubes for reagents (15 mL or 50 mL)
Standard 1.5 mL and 2 mL microfuge tubes
0.5 ml maximum recovery PCR tubes (e.g., Fisher cat. no. 14-222-294)
10 micron section from a formaldehyde-fixed paraffin-embedded tissue block affixed to a charged glass slide
Strong magnet stand (e.g., Miltenyi Macsimag separator, cat. no. 130-092-168)
Vortex mixer (e.g., VWR Vortex Genie)
Mini-centrifuge (e.g., VWR Model V).
PCR thermocycler (e.g., BioRad/MJ PTC-200)
Ethanol (Decon Labs, cat. no. 2716)
Distilled, deionized or RNAse-free H₂O (dH₂O e.g., Promega, cat. no. P1197)
Roche Complete Protease Inhibitor EDTA-Free tablets (Sigma-Aldrich, cat. no. 5056489001)
1 M Tris-HCl pH 8.0
1 M Hydroxyethyl piperazineethanesulfonic acid pH 7.5 (HEPES (Na+); Sigma-Aldrich, cat. no. H3375)
5 M Sodium chloride (NaCl; Sigma-Aldrich, cat. no. S5150-1L)
2 M Spermidine (Sigma-Aldrich, cat. no. S0266)
10% Triton® X-100 (Sigma-Aldrich, cat. no. X100)
Antibody to an epitope of interest. Because in situ binding conditions are more like those for immunofluorescence (IF) than those for ChIP, we suggest choosing IF-tested antibodies if CUT&RUN/Tag-tested antibodies are not available.
CUTAC control antibody to RNA Polymerase II Phospho-Rpb1 CTD Serine-5 phosphate (PolIIS5P, CST #13523 (D9N5I))
Secondary antibody, e.g., guinea pig a-rabbit antibody (Antibodies online cat. no. ABIN101961) or rabbit a-mouse antibody (Abcam cat. no. ab46540).
Protein A/G-Tn5 (pAG-Tn5) fusion protein loaded with double-stranded adapters with 19mer Tn5 mosaic ends (Epicypher cat. no. 15-1117)
Thermolabile Proteinase K (NEB P8111S)
1 M Magnesium Chloride (MgCl2; Sigma-Aldrich, cat. no. M8266-100G)
1 M [tris(hydroxymethyl)methylamino] propanesulfonic acid (TAPS) pH 8.5 (with NaOH)
N,N-dimethylformamide (Sigma-Aldrich cat. no. D-8654-250 mL)
Bio-mag Plus amine magnetic beads (Polysciences cat. no. 86001-10
NEBNext 2X PCR Master mix (ME541L)
PCR primers: 10 μM stock solutions of i5 and i7 primers with unique barcodes [Buenrostro, J. D. et al. Nature 523:486 (2015)] in 10 mM Tris pH 8. Standard salt-free primers may be used. We do not recommend Nextera or NEBNext primers.
10% Sodium dodecyl sulfate (SDS; Sigma-Aldrich, cat. no. L4509)
SPRI paramagnetic beads (e.g., HighPrep PCR Cleanup Magbio Genomics cat. no. AC-60500)

### Reagent Setup for Up to 16 Samples

1. Cross-link reversal buffer: Mix 800 μL 1 M Tris-HCl pH8.0, 200 L dH2O.

Triton®-Wash buffer: Mix 1 mL 1 M HEPES pH 7.5, 1.5 mL 5 M NaCl, 250 μl Triton®-X100 and 12.5 μl 2 M spermidine, bring the final volume to 50 mL with dH2O, and add 1 Roche Complete Protease Inhibitor EDTA-Free tablet. Store the buffer at 4° C. for up to 2 days.

Primary Antibody solution: Mix 17 μl RNA Polymerase II-Ser5p: (Cell Signaling Technologies (D9N5I) mAb #13523)+423 μl Triton-Wash buffer (1:25).

Secondary Antibody solution: Mix 17 μl guinea pig anti-rabbit (Antibodies Online) with 423 μL Triton-Wash buffer (1:25).

Protein A (G)-Tn5 solution: Mix 21 μl Protein A (G)-Tn5 (Epicypher cat. no. 15-1117) with 419 μL Triton®-Wash buffer (1:20).

CUTAC-DMF Tagmentation buffer: Mix 17.7 mL dH20, 4 mL N,N-dimethylformamide, 220 μl 1 M TAPS pH 8.5, and 110 μl 1 M MgCl2 (10 mM TAPS, 5 mM MgCl2, 20% DMF). Store the buffer at 4° C. for up to 1 week.

TAPS wash buffer: Mix 1 mL dH20, 10 μl 1 M TAPS pH 8.5, 0.4 μl 0.5 M EDTA (10 mM TAPS, 0.2 mM EDTA). Store at room temperature.

1% SDS/ProtK Release solution: (For 16 samples) Mix 10 μl 10% SDS and 1 μl 1 M TAPS pH 8.5 in 79 μl dH2O. Just before use add 10 μL Thermolabile Proteinase K (NEB cat. no. P8111S).

6% Triton® Mix 600 μl 10% Triton®-X100+400 μl dH2O. Store at room temperature.

### Deparaffinization in Hot Cross-Link Reversal Buffer (1.5 hr)

2. Place slides in cross-link reversal buffer in a slide holder that is filled to completely cover the slides. Place the holder in a water bath at 85 C and incubate for at least an hour. The paraffin will melt and float to the top. Remove slide holder to an ice-cold water bath to chill. Adding more solution to overfill will drain off any solid paraffin.

Note: Overnight 85 C incubations give similar results to 1 hr incubations. Be sure that the FFPE sections are affixed to a charged glass slide to avoid tissue loss during incubation.

Note: Overnight 85 C incubations give similar results to 1 hr incubations. Be sure that the FFPE sections are affixed to a charged glass slide to avoid tissue loss during incubation.

This protocol is for 16 samples but can be scaled up or down as needed. The example experiment shown in FIGS. 20-22 beginning with dry FFPE slides through sequencing-ready purified DNA libraries was accomplished in 1 day (~11 hours), but all of the steps can be lengthened with proper sealing to minimize evaporation. Overnight stopping points can be during any of the room temperature incubations by placing the plastic film-wrapped slides into a moist chamber and holding at 4° C.

3. Remove slides to Rinse Buffer in a slide holder.

4. For Option 1 (on-slide), continue with Step 5. For Option 2 (Magnetic Beads), skip to Step 22.

Option 1: On-slide FFPE-CUTAC incubation with primary antibody (1.5 hr)

5. For each slide, remove from slide holder, wick off excess liquid with a Kimwipe and place tissue-side up on a dark surface. Carefully pipette ~100 μl primary antibody solution over the tissue.

6. Cover the clear portion of the slide with a rectangle of plastic film using surface tension to spread the liquid, while excluding large bubbles and wrinkles. Place wrapped slides separated in a dry slide holder (FIG. 20).

Note: Any plastic wrap will seal adequately, but food service film on a heavy 2000 foot roll (e.g., Reynolds 912) is recommended for ease of pulling out wrap with both hands. Some kitchen wraps (Saran and Glad) are not as smooth and will be more difficult to work with. Before removing slides from the Rinse Buffer, use a razor to cut plastic film rectangles slightly wider and longer than the clear portion of the slide.

Note: Optionally, for incubating multiple slides with the same antibody or pA (G)-Tn5 solution, place the slides abutted against one another on the surface of a plastic box (FIG. 21). After adding the solution to the slide surfaces, lower stretched-out plastic film so that it makes contact with the liquid, then continue lowering so the meniscus moves over all of the samples.

Note: Any bubbles over tissue can be pushed to a section of tissue-free glass.

Note: Other antibodies that work with this protocol are H3K27ac (Abcam #4729) and RNA Polymerase II Serine-2,5p (Cell Signaling Technologies CST (D1G3K) mAb #13546.

7. Incubate horizontally for at least 1 hr.

8. Remove plastic wrap and gently rinse slide with 1 mL Triton®-Wash buffer. Option 1: Incubation with secondary antibody (1.5 hr)

9. Wick off excess liquid with a Kimwipe and place tissue-side up on a dark surface. Carefully pipette ~100 μl primary antibody solution over the tissue.

10. Cover the clear portion of the slide with a rectangle of plastic film using surface tension to spread the liquid, while omitting bubbles and folds. Place wrapped slides separated in a dry slide holder.

11. Incubate horizontally for at least 1 hr.

12. Remove plastic wrap and gently rinse slide with 1 mL Triton®-Wash buffer. Drain on paper towel or Kimwipe and place in a slide holder filled with Triton®-Wash buffer for 10 min.

Option 1: Binding Protein A (G)-Tn5 adapter complex (1.5 hr)

13. Remove from slide holder and wick off excess liquid with a Kimwipe. Place tissue-side up on a dark surface. Carefully pipette ~100 μl pA (G)-Tn5 solution over the tissue.

14. Cover the clear portion of the slide with a rectangle of plastic film using surface tension to spread the liquid, while omitting bubbles and folds. Place wrapped slides separated in a dry slide holder.

Note: When using other commercial sources of Protein A-Tn5 or Protein AG-Tn5 use the concentration recommended by the manufacturer for CUT&Tag. If using homemade fusion protein use the concentration recommended in the protocol for CUT&Tag, where the stock concentration may be higher (e.g., www.protocols.io/view/3xflag-patn5-protein-purification-and-meds-loading-j8nlke4e515r/v1).

15. Incubate horizontally for at least 1 hr.

16. Remove plastic wrap and gently rinse slide with 1 mL Triton®-Wash buffer. Drain on paper towel or Kimwipe and place in a slide holder filled with Triton®-Wash buffer for 10 min. Drain and place in a slide holder with Triton®-Wash buffer for 10 min.

17. Drain on paper towel or Kimwipe and place in a slide holder filled with 10 mM TAPS pH 8.5 for 10 min.

Option 1: Tagmentation and dissection (1.5 hr)

18. Remove slides and drain on paper towel or Kimwipe and place in a slide holder containing cold Tagmentation buffer.

19. Incubate 1 hr in a water bath at 55° C.

20. Remove each slide to a slide holder containing 10 mM TAPS pH 8.5 to hold.

21. Remove slide from slide holder, drain and use a Kimwipe to remove excess liquid from the top surface. Dissect or scrape using a total of no more than 5 μL 1% SDS/Thermolabile Proteinase K solution per PCR tube. For recovering all tissue from the slide dice and scrape with a safety razor blade. Proceed to Fragment release (Step 46). Option 2: FFPE-CUTAC using Biomag-amine beads

22. Remove slide from slide holder, drain and use a Kimwipe to remove excess liquid from the top surface. For recovering all tissue from the slide use a safety razor blade, first dicing the tissue, then scraping into a 2 ml tube containing 1 ml Triton®-wash buffer.

23. Add 1 μl Bio-mag Plus amine beads (48 mg/ml) per 8 final PCRs.

Note: Note the ~10-fold higher concentration of Bio-mag amine than ConA magnetic beads. Do not use ConA beads as they will bind bacteria contaminating FFPEs. Unlike ConA beads, Bio-Mag Plus amine beads are not activated and do not bind deparaffinized FFPE tissue shards as well. Amine beads require up-and-down full speed spins on a touch centrifuge (~3000xg) before placing on a magnet and decanting to avoid losses.

24. Pass through a 20-22 gauge 1″ needle using a Luer-lock glass syringe ~20 times to break up tissue. Divide and transfer into PCR tubes.

Note: Use firm plunges but not so hard as to cause overflowing. This procedure may result in foaming. To clear the foam, spin 3000xg for 1 minute, then vortexing will disperse the small shards of 10 μm thick tissue.

Option 2: Incubation with primary antibody (1.5 hr)

25. After a quick full spin, place the tubes on the magnet stand to clear and withdraw the liquid.

Note: The protocol for FFPEs is similar to CUT&Tag-direct Version 4, available at dx.doi.org/10.17504/protocols.io.x54v9mkmzg3e/v4, and can be performed in parallel with native or lightly cross-linked nuclei or whole cells.

26. Resuspend beads in 25 μl primary antibody solution followed by vortexing.

27. Incubate at least 1 hr on rotator or nutator at room temperature.

Option 2: Incubation with secondary antibody (1.5 hr)

28. After a quick full spin, place the tubes on the magnet stand to clear and withdraw the liquid.

29. Resuspend beads in 25 μl secondary antibody solution followed by vortexing.

30. Incubate at least 1 hr on rotator or nutator at room temperature.

31. After a quick full spin, place the tubes on the magnet stand to clear and remove and discard the supernatant with two successive draws, using a 20 μl tip with the pipettor set for maximum volume.

32. With the tubes still on the magnet stand, carefully add 500 μl of Wash buffer. The surface tension will cause the beads to slide up along the side of the tube closest to the magnet.

33. Slowly withdraw 460 μl of supernatant with a 1 mL pipette tip without disturbing the beads.

Note: To remove the supernatant, set the pipettor to 460 μl, and keep the plunger depressed while lowering the tip to the bottom. The liquid level will rise to near the top completing the wash. Then ease off on the plunger until the liquid is withdrawn and remove the pipettor. During liquid removal, the surface tension will drag the beads down the tube. A small drop of liquid that is left behind will be removed in the next step.

Note: Bead-bound shards from FFPEs stick to the sides of low-bind PCR tubes, which is especially conspicuous after Wash buffer removal and vortexing is not sufficient to wet them. Therefore, tubes should be mixed by inversion after vortexing.

34. After a quick full spin, place the tubes back into the magnet stand and remove the remaining supernatant with a 20 μl pipettor multiple times if necessary, to remove the entire supernatant without disturbing the beads. Proceed immediately to the next step.

Option 2: Binding Protein A (G)-Tn5 adapter complex

35. Mix pAG-Tn5 pre-loaded adapter complex in Triton®-Wash buffer following the manufacturer's instructions (e.g., 1:20 for EpiCypher pAG-Tn5).

Note: This protocol is not recommended for “homemade” pA-Tn5 following our purification protocol, because the contaminating E. coli DNA will be preferentially tagmented relative to the less accessible FFPE DNA under the stringent 55° C. conditions used here. If homemade pA-Tn5 is used, it is important to minimize the amount added (≤1:200).

36. Pipette in 25 μl per sample of the pA (G)-Tn5 mix followed by vortexing.

37. After a quick spin, place the tubes on a Rotator or Nutator at room temperature for 1 hr or 4° C. overnight.

38. After incubating in the rotator, perform a quick full spin and place the tubes in the magnet stand.

39. Carefully remove the supernatant using a 20 μl pipettor as in Step 31.

40. With the tubes still on the magnet stand, add 500 μl of the Triton®-Wash buffer.

41. Slowly withdraw 460 μl with a 1 ml pipette tip without disturbing the beads as in Step 33.

42. After a quick full spin, place the tubes back on the magnet stand and remove and discard the supernatant with a 20 μL pipettor using multiple draws.

### Option 2: Tagmentation (1.5 hr)

43. Resuspend the bead/FFPE pellet in 50 μl CUTAC-DMF tagmentation solution (5 mM MgCl₂, 10 mM TAPS, 20% DMF, 0.05% Triton®-X) while vortexing. Incubate 1 hr 55° C. in thermocycler.

Note: N,N-dimethylformamide is a dehydrating compound resulting in improved tethered Tn5 accessibility and library yield. Conditions used for FFPEs are the most stringent tested in Henikoff S, Henikoff J G, Kaya-Okur H S, Ahmad K. Efficient chromatin accessibility mapping in situ by nucleosome-tethered tagmentation. Elife. 2020 Nov. 16;9: e63274. doi: 10.7554/eLife.63274-FIG. 3-figure supplement 2.

44. Place tubes on a magnet stand and remove and discard the supernatant with a 20 μL pipettor using multiple draws then resuspend the beads in 50 μL TAPS wash and mix by vortexing.

45. Add 5 μL SDS/Proteinase K, vortex, spin, revortex and spin. Proceed to Fragment release (Step 46).

### Fragment Release (1.5 hr)

46. After a full speed spin, incubate at 37° C. for 30 min and 58° C. for 30 min (programmed in succession in a PCR cycler with a heated lid) to release pA-Tn5 from the tagmented DNA.

### PCR (1 hr)

47. To the PCR tube containing the bead slurry add 15 μl of Triton® neutralization solution+2 μl of 10 μM Universal or barcoded i5 primer+2 μl of 10 μM uniquely barcoded i7 primers, using a different barcode for each sample. Vortex on full speed and place tubes in the metal tube holder on ice.

Note: Indexed primers are described by Buenrostro, J. D. et al. Single-cell chromatin accessibility reveals principles of regulatory variation. Nature 523:486 (2015). Nextera or NEB primers are not recommended, which might not anneal efficiently using this PCR protocol.

48. Add 25 μl NEBnext (non-hot-start), vortex to mix, and perform a quick spin. Place the tubes immediately in the thermocycler and proceed immediately with the PCR.

49. Begin the cycling program with a heated lid on the thermocycler: Cycle 1:58° C. for 5 min (gap filling) Cycle 2:72° C. for 5 min (gap filling) Cycle 3:98° C. for 5 min Cycle 4:98° C. for 10 sec Cycle 5:63° C. for 30 sec Cycle 6:72° C. for 1 min Repeat Cycles 4-6 11 times Hold at 8° C.

Note: CUT&Tag uses short 2-step 10 sec cycles to favor amplification of nucleosomal and smaller fragments. However, after cross-link reversal, DNA in FFPEs are small and PCR amplicon sizes <120 bp are recommended (Do and Dobrovic, Clin. Chem. 61 (1): 64-71 (2015)), which obviates the need to minimize the contribution of large DNA fragments. Insertion of a 1 min 72° C. extension and lengthening of the 63° C. annealing time from 10 sec to 30 sec results in better read-through of damaged DNA by Taq polymerase, resulting in a higher fraction of mappable reads than using the 2-step cycle favored for CUT&Tag and CUTAC.

Note: No more than 12 cycles are recommended. Do not add extra PCR cycles to see a signal by capillary gel electrophoresis (e.g., Tapestation). Extra PCR cycles reduce the complexity of the library and may favor contaminating bacterial DNA from the paraffin (FIG. 20).

Post-PCR Clean-up (30 min)

50. After the PCR program ends, remove tubes from the thermocycler and add 65 μL of SPRI beads (ratio of 1.3 μL of SPRI beads to 1 μL of PCR product). Mix by pipetting up and down.

51. Let sit at room temperature 5-10 min.

52. Place on the magnet stand for a few minutes to allow the solution to clear.

53. Remove and discard the supernatant.

54. Keeping the tubes in the magnet stand, add 200 μL of 80% ethanol.

55. Completely remove and discard the supernatant.

56. Repeat Steps 54 and 55.

57. Perform a quick spin and remove the remaining supernatant with a 20 μl pipette, avoiding air drying the beads by proceeding immediately to the next step.

58. Remove from the magnet stand, add 22 μl 10 mM Tris-HCl pH 8 and vortex at full speed. Let sit for 5 min to 1 hr.

59. Place on the magnet stand and allow to clear.

60. Remove the liquid to a fresh 1.5 mL tube with a pipette, avoiding transfer of beads.

### Tapestation Analysis and DNA Sequencing

61. Determine the size distribution and concentration of libraries by capillary electrophoresis using an Agilent 4200 TapeStation with D1000 reagents or equivalent.

Note: Quantification by Tapestation was used to estimate library concentration and dilute each library to 2 nM (or the concentration specified for Illumina library submission at the sequencing core that will process your sample) before pooling based on fragment molarity in the 175-500 bp range.

Note: Library samples from a single slide should be pooled using equal volumes to simplify comparisons between them. For direct comparisons between multiple slides processed in parallel using the same antibody, use equal volumes for all samples derived from them.

62. Mix barcoded libraries to achieve equal representation as desired aiming for a final concentration as recommended by the manufacturer. After mixing, perform an SPRI bead cleanup if needed to remove any residual PCR primers.

63. Perform paired-end Illumina sequencing on the barcoded libraries following the manufacturer's instructions.

Note: Currently, paired-end 50×50 sequencing on an Illumina Next-Seq was used, obtaining ~400 million total mapped reads, or ~4 million per sample when there are 96 samples mixed to obtain approximately equal molarity.

### Data Processing and Analysis

64. Align paired-end reads to hg19 using Bowtie2 version 2.3.4.3 with options:--end-to-end--very-sensitive--no-unal--no-mixed--no-discordant--phred33-I 10-X 700. For mapping E. coli carry-over fragments, we also use the—-no-overlap--no-dovetail options to avoid possible cross-mapping of the experimental genome to that of the carry-over E. coli DNA that is used for calibration. Tracks are made as bedgraph files of normalized counts, which are the fraction of total counts at each basepair scaled by the size of the hg19 genome.

65. The CUT&Tag Data Processing and Analysis Tutorial on Protocols.io available at protocols.io/view/cut-amp-tag-data-processing-and-analysis-tutorial-e6nvw93x7gmk/v1 provides step-by-step guidance for mapping and analysis of CUT&Tag sequencing data.

Most data analysis tools used for ChIP-seq data, such as bedtools, Picard and deepTools, can be used on CUT&Tag data. Analysis tools designed specifically for CUT&RUN/Tag data include the SEACR peak caller also available as a public web server and CUT&RUNTools.

### Example 3. CUTAC for FFPEs V.3

### Materials

Chilling device (e.g., metal heat blocks on ice or cold packs in an ice cooler)
Pipettors (e.g., Rainin Classic Pipette 1 mL, 200 μL, 20 μL, and 10 μL)
Kimble Kontes Pellet Pestle Motor (DWK Life Sciences cat no. 749540-0000)
Disposable pestles (Fisher cat. on. 12-141-364)
Disposable tips (e.g., Rainin 1 mL, 200 μL, 20 μL)
Disposable centrifuge tubes for reagents (15 mL or 50 mL)
Standard 1.5 mL and 2 mL microfuge tubes
0.5 ml maximum recovery PCR tubes (e.g., Fisher cat. no. 14-222-294)
10 micron section from a formaldehyde-fixed paraffin-embedded tissue block affixed to a charged glass slide
Strong magnet stand (e.g., Miltenyi Macsimag separator, cat. no. 130-092-168)
Vortex mixer (e.g., VWR Vortex Genie)
Mini-centrifuge (e.g., VWR Model V).
PCR thermocycler (e.g., BioRad/MJ PTC-200)
Bio-Mag Plus amine magnetic beads (48 mg/ml, Polysciences cat. no. 86001-10).
Dilute 1:10 with 10 mM Tris pH8/1 mM EDTA for use.
Pierce glutathione magnetic beads (Fisher cat. no. 88822)
Ethanol (Decon Labs, cat. no. 2716)
Distilled, deionized or RNAse-free H2O (dH2O e.g., Promega, cat. no. P1197)
Roche Complete Protease Inhibitor EDTA-Free tablets (Sigma-Aldrich, cat. no. 5056489001)
1 M Tris-HCl pH 8.0
1 M Hydroxyethyl piperazineethanesulfonic acid pH 7.5 (HEPES (Na+); Sigma-Aldrich, cat. no. H3375)
5 M Sodium chloride (NaCl; Sigma-Aldrich, cat. no. S5150-1L)
2 M Spermidine (Sigma-Aldrich, cat. no. S0266)
10% Triton® X-100 (Sigma-Aldrich, cat. no. X100)
0.5 M EDTA pH 8
10% Sodium azide (caution: toxic)
Antibody to an epitope of interest. Because in situ binding conditions are more like those for immunofluorescence (IF) than those for ChIP, we suggest choosing IF-tested antibodies if CUT&RUN/Tag-tested antibodies are not available.
CUTAC control antibody to RNA Polymerase II Phospho-Rpb1 CTD Serine-5 phosphate (PolIIS5P, CST #13523 (D9N5I))
Secondary antibody, e.g., guinea pig a-rabbit antibody (Antibodies online cat. no. ABIN101961) or rabbit a-mouse antibody (Abcam cat. no. ab46540)
Protein A/G-Tn5 (pAG-Tn5) fusion protein loaded with double-stranded adapters with 19mer Tn5 mosaic ends (Epicypher cat. no. 15-1117)
Thermolabile Proteinase K (NEB P8111S)
1 M Magnesium Chloride (MgCl2; Sigma-Aldrich, cat. no. M8266-100G)
1 M [tris(hydroxymethyl)methylamino] propanesulfonic acid (TAPS) pH 8.5 (with NaOH)
N,N-dimethylformamide (Sigma-Aldrich cat. no. D-8654-250 mL)
NEBNext 2X PCR Master mix (ME541L)
PCR primers: 10 μM stock solutions of i5 and i7 primers with unique barcodes [Buenrostro, J. D. et al. Nature 523:486 (2015)] in 10 mM Tris pH 8. Standard salt-free primers may be used. We do not recommend Nextera or NEBNext primers.
10% Sodium dodecyl sulfate (SDS; Sigma-Aldrich, cat. no. L4509)
SPRI paramagnetic beads (e.g. HighPrep PCR Cleanup Magbio Genomics cat. no. AC-60500)

1. Cross-link reversal buffer Mix 8 ml 1 M Tris-HCl pH8.0, 2 ml dH2O and 4 μl 0.5 mM EDTA.

Rinse buffer (Option 1) Mix 1 mL 1 M HEPES pH 7.5 and 1.5 mL 5 M NaCl, and bring the final volume to 50 mL with dH2O.

Triton®-Wash buffer Mix 1 mL 1 M HEPES pH 7.5, 1.5 mL 5 M NaCl, 250 μl 10% Triton®-X100, 12.5 μl 2 M spermidine and 20 μl 0.5 M EDTA, bring the final volume to 50 mL with dH2O, and add 1 Roche Complete Protease Inhibitor EDTA-Free tablet. Store the buffer at 4° C. for up to 2 days.

Note To completely prevent bacterial contamination during long incubations and storage of Triton®-wash buffer, add Sodium azide to 0.02% (100 μl 10%->50 mL), but handle this toxic chemical carefully, and wear a mask when weighing it out.

Primary antibody solution Mix 17 μl RNA Polymerase II-Ser5p: (Cell Signaling Technologies (D9N5I) mAb #13523)+423 μl Triton®-Wash buffer (1:25).

Secondary antibody solution Mix 17 μl guinea pig anti-rabbit (Antibodies Online) with 423 μL Triton®-Wash buffer (1:25).

Protein A (G)-Tn5 solution Mix 21 μl Protein A (G)-Tn5 (Epicypher cat. no. 15-1117) with 419 μL Triton®-Wash buffer (1:20).

CUTAC-DMF Tagmentation buffer Mix 17.7 mL dH20, 4 mL N,N-dimethylformamide, 220 μl 1 M TAPS pH 8.5, and 110 μl 1 M MgCl2 (10 mM TAPS, 5 mM MgCl2, 20% DMF). Store the buffer at 4° C. for up to 1 week.

TAPS wash buffer Mix 1 mL dH20, 10 μl 1 M TAPS pH 8.5, 0.4 μl 0.5 M EDTA (10 mM TAPS, 0.2 mM EDTA). Store at room temperature.

1% SDS/ProtK Release solution (For 16 samples) Mix 10 μl 10% SDS and 1 μl 1 M TAPS pH 8.5 in 79 μl dH2O. Just before use add 10 μL Thermolabile Proteinase K (NEB cat. no. P8111S).

6% Triton® Mix 600 μl 10% Triton®-X100+400 μl dH2O. Store at room temperature.

Option 1: On-slide FFPE-CUTAC deparaffinization in hot cross-link reversal buffer.

2. Place slides in cross-link reversal buffer in a slide holder that is filled to completely cover the slides. Place the holder in a water bath at 85-90 C and incubate for at least an hour. The paraffin will melt and float to the top. Remove slide holder to an ice-cold water bath to chill. Adding more solution to overfill will drain off any solid paraffin.

3. Remove slides to Rinse Buffer in a slide holder.

4. For Option 1 (on-slide), continue with Step 5. For Option 2 (Magnetic Beads), skip to Step 22.

Option 1 (continued): On-slide FFPE-CUTAC Incubation with primary antibody

5. For each slide, remove from slide holder, wick off excess liquid from the glass surface with a Kimwipe (without touching the tissue) and place tissue-side up on a dark surface for visibility. Carefully pipette ~50 μl primary antibody solution over the tissue.

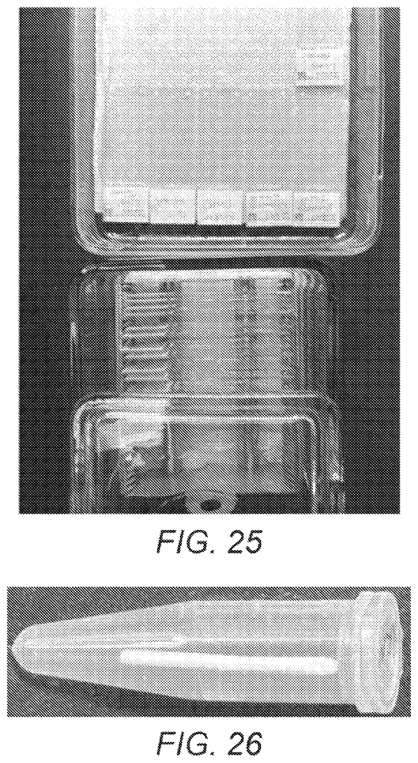

6. Cover the clear portion of the slide with a rectangle of plastic film (or a square for small tissue sections) using surface tension to spread the liquid, while excluding large bubbles and wrinkles. Place wrapped slides separated in a dry slide holder (FIG. 20) or in the rack of a staining dish, which can be used as a “moist chamber” (FIG. 25).

Note: Any bubbles over tissue can be pushed to a section of tissue-free glass.

Note: Other antibodies that work with this protocol are H3K27ac (Abcam #4729) and RNA Polymerase II Serine-2,5p (Cell Signaling Technologies CST (D1G3K) mAb #13546. Antibodies to histone methylations have failed, and unsatisfactory results have been obtained using an antibody to CTCF.

Note: Any plastic wrap will seal adequately, but food service film on a heavy 2000 foot roll (e.g., Reynolds 912) is recommended for ease of pulling out wrap with both hands. Some kitchen wraps (Saran and Glad) are not as smooth and will be more difficult to work with. Before removing slides from the Rinse Buffer, use a razor to cut plastic film rectangles slightly wider and longer than the clear portion of the slide.

7. Incubate at room temperature for at least 1 hr.

8. Remove plastic wrap and gently rinse slide by pipetting 1 mL Triton®-Wash buffer dropwise over the top of the slide.

Option 1 (continued): Incubation with secondary antibody (1.5 hr)

9. Wick off excess liquid with a Kimwipe and place tissue-side up on a dark surface. Carefully pipette ~50 μl secondary antibody solution over the tissue.

10. Cover the clear portion of the slide with a rectangle of plastic film using surface tension to spread the liquid, while omitting bubbles and folds. Place wrapped slides separated in a dry slide holder.

11. Incubate at room temperature for at least 1 hr.

12. Remove plastic wrap and gently rinse slide 1-2 times with 1 mL Triton®-Wash buffer.

Option 1 (continued): Binding Protein A (G)-Tn5 adapter complex (1.5 hr)

13. Remove from slide holder and wick off excess liquid with a Kimwipe. Place tissue-side up on a dark surface. Carefully pipette ~50 μl pA (G)-Tn5 solution over the tissue.

14. Cover the clear portion of the slide with a rectangle of plastic film using surface tension to spread the liquid, while omitting bubbles and folds.

15. Incubate at room temperature for at least 1 hr.

16. Remove plastic wrap and gently rinse slide 1-2 times with 1 mL Triton®-Wash buffer. Drain on paper towel or Kimwipe and place in a slide holder filled with Triton®-Wash buffer for 10 min. Drain and place in a slide holder with Triton®-Wash buffer for 10 min.

17. Drain on paper towel and wick off excess liquid with a Kimwipe and place in a slide holder filled with 10 mM TAPS pH 8.5 for 10 min.

Option 1 (continued): Tagmentation and dissection (1.5 hr)

18. Remove slides and drain on paper towel or Kimwipe and place in a slide holder containing cold Tagmentation buffer.

19. Incubate 1 hr in a water bath at 55° C.

20. Remove each slide to a slide holder containing 10 mM TAPS pH 8.5 to hold.

21. Remove slide from slide holder, drain and use a Kimwipe to remove excess liquid from the top surface. Dissect or scrape using a total of no more than 5 μL 1%

SDS/Thermolabile Proteinase K solution per PCR tube. For larger tissue amounts, use more SDS/TLProtK solution and divide up into PCR tubes such that no more than 5 μL is deposited into each tube. To recover all tissue from the slide dice and scrape with a safety razor blade. Vortex and centrifuge to compact beads in the bottom of the tube and proceed to Fragment Release (Step 48).

Note: For dissection into a PCR tube, first add 2 μl to the tube, then 2 μl to the desired section of tissue using the pipette tip to spread the solution and loosen the tissue from the slide. Use a #3-5 jeweler's forceps and a scalpel or razor blade to scrape each section into a pile and deposit it into the PCR tube. A 1 μl aliquot of the solution can be used to remove the remaining tissue from the slide into the tube.

Note: Working quickly reduces the chance that tissue will dry out during dissection. However, no loss of data quality was noticed when tissue dries before being wetted with SDS/Proteinase K solution.

### Option 2: FFPE-CUTAC Using Beads: Deparaffinization in Mineral Oil and Cross-Link Reversal Buffer

22. FFPE slide or curl: Scrape all or part of a 10 μm FFPE slide (FIGS. 20, 22 and 25) or a “curl” (FIG. 26) into a 1.7 ml tube (e.g., MCT-175-C), add 200 μL mineral oil. Vortex, spin, and place in a 90° C. water bath for 5 min. While still warm vortex to fully suspend the paraffin and spin on full.

Note: The Option 2 protocol is for 16 samples but can be scaled up or down as needed. Sequencing-ready purified DNA libraries can be obtained in one long day (~10 hours), but any of the 1 hr antibody or pAG-Tn5 incubations can be extended to a few hours at room temperature or at 4-8° C. overnight.

Note: Vortex hard to mix, but in some steps a “quick vortex” is used. With a touch mini-centrifuge, “spin on full” is just up to full speed then down, whereas “quick spin” is only to remove liquid from the cap and down from the sides.

Note: Curls are thin sections that are released from the microtome without being affixed to slides and curl up to form a tight rod.

Using a blue pestle attached to a pestle motor place the pestle into the bottom of the tube, start the motor and homogenize with short up-and-down motions for ~20 sec.

24. Add 200 μl warm Cross-link reversal buffer, then 6 μl 1:10-diluted Biomag Plus amine beads into the bottom (aqueous) layer, vortex and homogenize ~20 sec with the motorized pestle.

25. Add 800 μl warm Cross-link reversal buffer and vortex to mix, spin on full and replace in the 90° C. water bath. Incubate ≥1 hr.

26. Remove from water bath, mix by hard vortexing and spin on full. Very carefully remove the top (oil) layer without disturbing the interface, where there will be trapped tissue and beads, leaving behind a thin oil layer above the meniscus.

27. Add 500 μl mineral oil, mix by inversion (do not vortex), spin on full and carefully remove the mineral oil layer leaving behind a thin oil layer. Repeat with a second 500 μl mineral oil wash. Respin to clear tube sides and pipet off excess oil, leaving behind a thin oil layer above the meniscus.

28. Add 2.4 μL (undiluted) Pierce glutathione beads to bottom of tube avoiding the mineral oil on the surface. Mix by inversion.

29. Do a quick spin and place on the magnet stand. When clear carefully remove the supernatant using a 200 μL low-bind pipette tip.

30. Add 1 ml Triton®-Wash buffer and vortex followed by a quick spin, and divide into two or more PCR tubes. The following assumes two PCR tubes per scrape or curl, one for RNAPII-Ser5p and one for H3K27ac, but for smaller aliquots volumes should be adjusted to maintain the concentration of reagents.

31. Place tubes on magnet stand and carefully remove supernatant using a low-bind 200 μL pipette tip.

Option 2 (continued): Incubation with primary antibody

32. Resuspend beads in 100 μl primary antibody solution followed by vortexing.

33. Incubate at least 1 hr on rotator or nutator at room temperature.

Option 2 (continued): Incubation with secondary antibody

34. After a quick spin, place the tubes on the magnet stand to clear and withdraw and discard the antibody supernatant using a 200 μL low-bind pipette tip.

35. Resuspend beads in 100 μl secondary antibody solution followed by vortexing.

36. Incubate at least 1 hr on rotator or nutator at room temperature.

37. After a quick spin, place the tubes on the magnet stand and withdraw and discard the antibody supernatant using a 200 μL low-bind pipette tip.

38. While on the magnet stand, slowly drip in 500 μl of Triton®-Wash buffer. Carefully withdraw and discard the wash supernatant using a 200 μL low-bind pipette tip. Proceed immediately to the next step.

Option 2 (continued): Binding Protein A (G)-Tn5 adapter complex

39. Mix pAG-Tn5 pre-loaded adapter complex in Triton®-Wash buffer following the manufacturer's instructions (e.g., 1:20 for EpiCypher pAG-Tn5).

40. Add 100 μl pA (G)-Tn5 mix followed by vortexing. Place the tubes on a rotator or nutator at room temperature for ≥1 hr.

41. After a quick spin, place the tubes on the magnet stand and withdraw and discard the pA (G)-Tn5 supernatant using a 200 μl low-bind pipette tip.

42. While on the magnet stand, slowly drip in 500 μl of Triton®-Wash buffer. Carefully withdraw and discard the wash supernatant using a 200 μl low-bind pipette tip.

43. While on the magnet stand, add 200 μl TAPS wash. Withdraw and discard the TAPS wash supernatant using a 200 μL low-bind pipette tip. Proceed immediately to the next step.

Option 2 (continued): Tagmentation

44. Resuspend the bead/FFPE pellet in 100 μl CUTAC-DMF tagmentation solution (5 mM MgCl₂, 10 mM TAPS, 20% DMF, 0.05% Triton®-X100) while vortexing. Incubate at 55° C. for 1 hr in a thermocycler.

45. After a quick full centrifugation, place the tubes on a magnet stand and withdraw and discard the Tagmentation buffer supernatant using a 200 μl low-bind pipette tip.

46. While on the magnet stand, add 100 μl TAPS wash. Withdraw and discard the TAPS wash supernatant using a 200 μl low-bind pipette tip.

47. Add 10 μl 1% SDS/Thermolabile Proteinase K solution per PCR tube. Vortex, quick spin and proceed to Fragment Release (Step 48).

### Fragment Release and PCR

48. Incubate at 37° C. for 30 min and 58° C. for 30 min (programmed in succession in a PCR cycler with a heated lid) to release pA-Tn5 from the tagmented DNA. Open the tubes and add 15 μL 6% Triton®-X100, close and incubate at 37° C. for 30 min on the cycler.

49. Add 2 μl of 10 μM Universal or barcoded i5 primer+2 μl of 10 μM barcoded i7 primers, using a different barcode pair for each sample. Vortex on full and place tubes in the metal tube holder on ice.

50. Add 25 μl NEBnext (non-hot-start), vortex to mix, and perform a quick spin. Place the tubes in the thermocycler and proceed immediately with the PCR.

51. Begin the cycling program with a heated lid on the thermocycler: Cycle 1:58° C. for 5 min (gap filling) Cycle 2:72° C. for 5 min (gap filling) Cycle 3:98° C. for 5 min Cycle 4:98° C. for 10 sec Cycle 5:63° C. for 30 sec Cycle 6:72° C. for 1 min Repeat Cycles 4-6 11 times Hold at 8° C.

Post-PCR Clean-up (30 min)

52. After the PCR program ends, remove tubes from the thermocycler, vortex to resuspend, and add 130 μL of SPRI beads (ratio of 1.3 μL of SPRI beads to 1 μL of PCR product). Mix by pipetting up and down.

53. Let sit at room temperature 5-10 min.

54. Place on the magnet stand for a few minutes to allow the solution to clear.

55. Remove and discard the supernatant.

56. Keeping the tubes in the magnet stand, add 400 μL of 80% ethanol.

57. Completely remove and discard the supernatant.

58. Repeat Steps 56 and 57.

59. Perform a quick spin and remove the remaining supernatant, avoiding air drying the beads by proceeding immediately to the next step.

60. Remove from the magnet stand, add 22 μl 10 mM Tris-HCl pH 8, vortex and quick spin. Let sit for at least 5 min to elute the DNA.

61. Place on the magnet stand and allow to clear.

62. Remove the liquid to a fresh 1.5 mL tube with a pipette, avoiding transfer of beads.

### Tapestation Analysis and DNA Sequencing

63. Determine the size distribution and concentration of libraries by capillary electrophoresis using an Agilent 4200 TapeStation with D1000 reagents or equivalent.

64. Mix barcoded libraries to achieve equal representation as desired aiming for a final concentration as recommended by the manufacturer. After mixing, perform an SPRI bead cleanup if needed to remove any residual PCR primers.

65. Perform paired-end Illumina sequencing on the barcoded libraries following the manufacturer's instructions.

### Data Processing and Analysis

66. Align paired-end reads to hg19 using Bowtie2 version 2.3.4.3 with options:--end-to-end--very-sensitive--no-unal--no-mixed--no-discordant--phred33-I 10-X 700. For mapping E. coli carry-over fragments, the—-no-overlap--no-dovetail options were also used to avoid possible cross-mapping of the experimental genome to that of the carry-over E. coli DNA that is used for calibration. Tracks are made as bedgraph files of normalized counts, which are the fraction of total counts at each basepair scaled by the size of the hg19 genome.

67. The CUT&Tag Data Processing and Analysis Tutorial on Protocols.io available at protocols.io/view/cut-amp-tag-data-processing-and-analysis-tutorial-e6nvw93x7gmk/v1 provides step-by-step guidance for mapping and analysis of CUT&Tag sequencing data. Most data analysis tools used for ChIP-seq data, such as bedtools, Picard and deepTools, can be used on CUT&Tag data. Analysis tools designed specifically for CUT&RUN/Tag data include the SEACR peak caller also available as a public web server and CUT&RUNTools.

### Example 4. RNA Polymerase II Hypertranscription in Cancer FFPE Samples

Hypertranscription is global upregulation of transcription, which is common in rapidly proliferating cells. In cancer, hypertranscription confers a worse prognosis, independent of somatic mutation burden, tumor ploidy, tumor stage, patient gender, age, or tumor subtype (Zatzman et al. Sci Adv 8 (47): eabn0238 (2022). Hypertranscription has thus far been assayed indirectly using RNA-seq data calibrated in variety of ways, but none have been suitable for clinical application.

This example shows FFPE-CUTAC can be used to directly map hypertranscription at regulatory elements throughout the mouse genome, revealing that the degree of hypertranscription varies between genetically identical tumors and for some is not observed at all. When applied to tumor and adjacent normal 5 micron ~1 cm² human FFPE sections from seven anonymous individual human tumors, FFPE-CUTAC analyzed for hypertranscription identified dozens of strongly hypertranscribed loci in common among the tumors. Strikingly, in two of the seven individual tumors broad increases of RNAPII within chromosome 17q12-21, which includes the ERBB2 (HER2) locus, were observed. These evident HER2 amplifications were punctuated with broad hypertranscribed regions, suggestive of linkage disequilibrium during tumor evolution (17, 18). Our data suggest that selective sweeps of direct regulators of RNAPII, including the CDK12 kinase, contribute to the poor prognosis associated with hypertranscription. The ability of FFPE-CUTAC to categorize tumors with sparse material, precisely localize patterns of regulatory element hypertranscription, and map megabase-sized regions of amplification interspersed with smaller regions of likely clonal selection, makes it an attractive platform for general personalized medicine applications.

### Results

### Hypertranscription in Mouse Brain Tumors

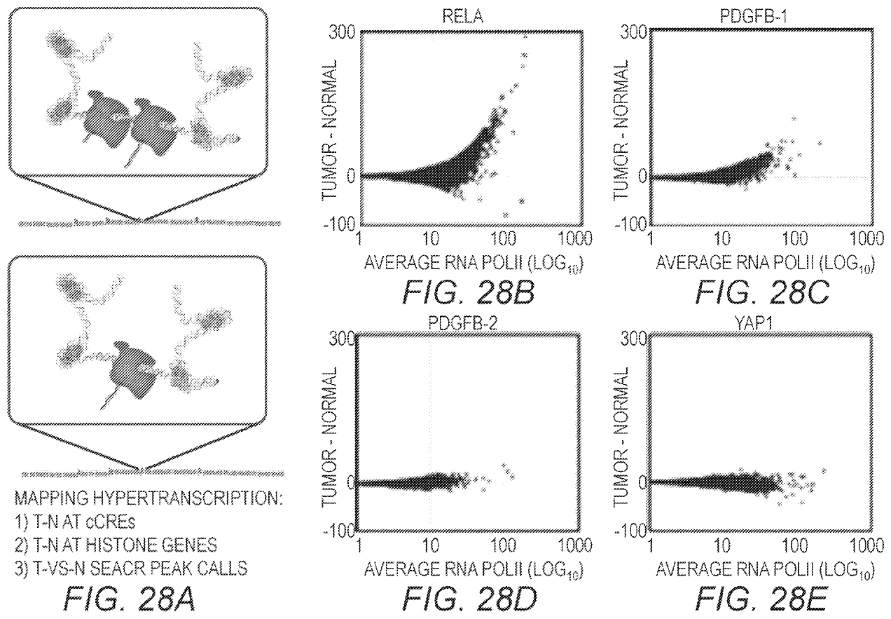

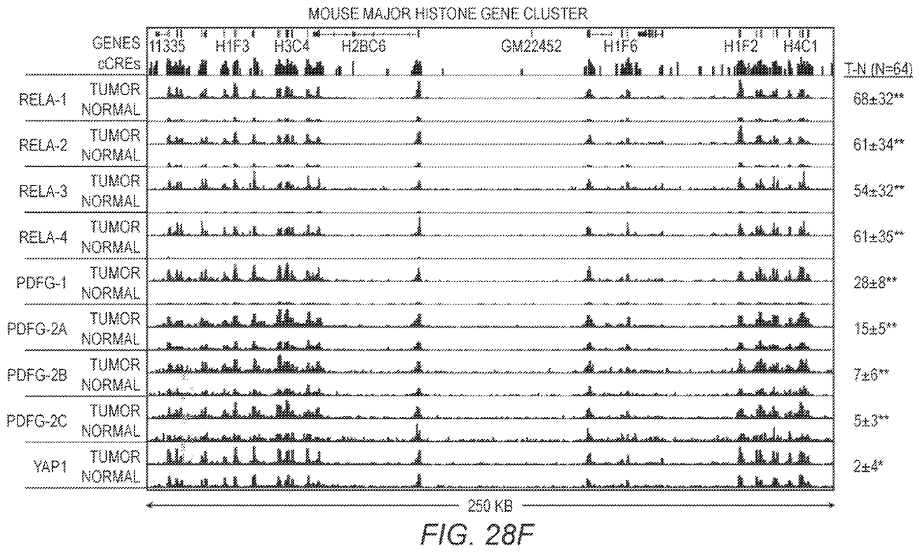

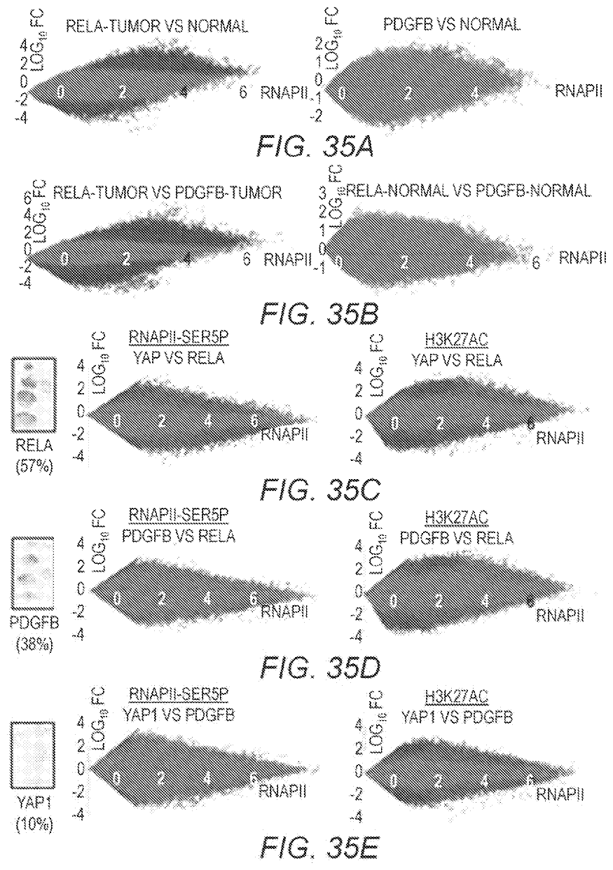

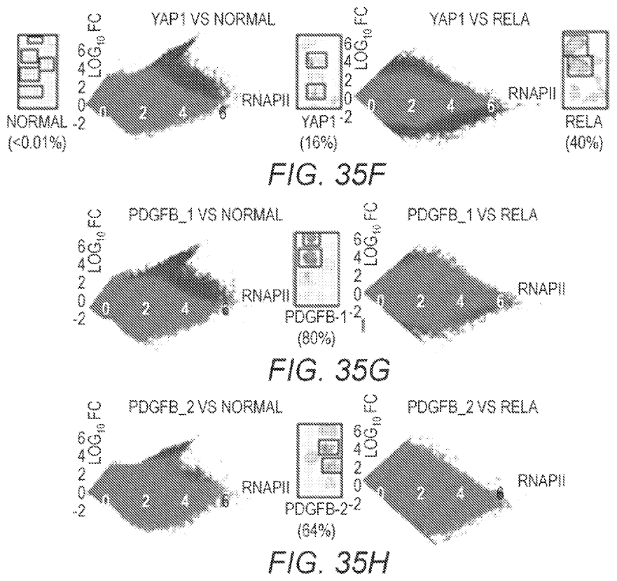

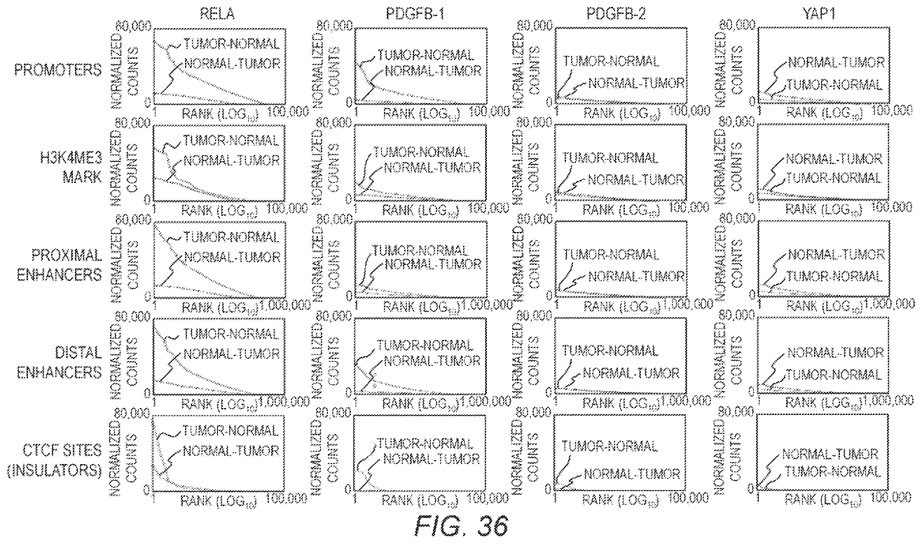

In the earlier FFPE-CUTAC study (13), described in Example 1, it was observed that significantly upregulated cCREs were more frequent than downregulated cCREs in mouse brain tumors with different transgene drivers: a ZFTA-RELA (RELA) transcription factor gene fusion driving an ependymoma (19), a YAP1-FAM118b (YAP1) transcriptional co-activator gene fusion driving an ependymoma (20), and overexpression of the tyrosine-kinase active PDGFB ligand driving a glioma (21). Upregulation bias based on RNAPII log 2 (fold-change) plotted on the y-axis as a function of log 10 (average signal) on the x-axis (MA plot) is observed in pooled data from several experiments in which tumor-rich sections were separated from normal sections (FIGS. 35A-35E). The upregulation bias is much greater for RELA than for PDGFB, and to further understand these differences and to eliminate sample-to-sample variability, on-slide dissection data from single FFPE slides representing normal mouse brain, RELA and YAPI tumors and PDGFB tumors from two genetically identical mice were examined. Unexpectedly, upregulation based on foldchange showed little relationship to the percentage of tumor in the sample as determined by counting cells stained for tumor transgene expression. For example, YAPI tumor sections averaged 16% tumor cells and showed similar upregulation bias to the PDGFB-1 sections with 80% tumor cells and stronger upregulation bias than the PDGFB-2 sections with 64% tumor cells (FIGS. 35F-35H, right panels) and all three showed weaker upregulation versus the RELA tumor sections with 40% tumor cells (FIGS. 35F-35H, left panels). The fold-change ratio of tumor: normal does not distinguish between a weak signal increasing to moderate strength and a moderate signal increasing to high strength. However, significant upregulation differences increased in log 10 (RNAPII) signal with reduced fold-change (red dots approaching the x-axis left to right) were noted, which suggests that the major differences in RNAPII upregulation in tumors result from significant increases in already high transcription levels, i.e., hypertranscription. The RNAPII FFPE-CUTAC assay is well-suited to detect minor absolute differences in regulatory element RNAPII occupancy (FIG. 28A), unlike RNA readouts that require calibration to the DNA template. For each tumor and normal sample, the number of mapped fragments spanning each base-pair in a cCRE scaled to the mouse genome were counted and the number of counts over that cCRE averaged. To sensitively detect hypertranscription, we plotted Tumor minus Normal counts on the y-axis versus average RNAPII signal (Bland-Altman plot (22)) plotted on a log 10 scale on the x-axis for clarity. This revealed clear hypertranscription for RELA (FIG. 28B). Interestingly, PDGFB tumors differed in hypertranscription, strongly in PDGFB-1 (FIG. 28C) and very weakly in PDGFB-2 (FIG. 28D), whereas YAP1 showed weak hypotranscription. To determine whether hypertranscription assayed by RNAPII abundance over cCREs is specific to any particular class of regulatory element(s), the data presented in FIGS. 28B-28E was divided into the five ENCODE-annotated categories: Promoters (24,114), H3K4me3-marked cCREs (10,538), Proximal Enhancers (108,474), Distal Enhancers (211,185) and CTCF cCREs (24,072). It was observed that the five RNAPII hypertranscription profiles are highly consistent with one another (FIG. 364), which suggests that RNAPII abundance differences between tumors and normal brain affect all regulatory element classes. To verify that these differences in global upregulation of cCREs are related to tumor growth, the profiles of the replication-coupled histone genes were examined, which provides an independent measure of cell proliferation. In total, these small single-exon genes produce RNAPII-dependent U7-processed single-exon mRNAs during S-phase to encode for the histones that package the entire genome in nucleosomes, and so the abundance of RNAPII at these histone gene loci provides a proxy for steady-state DNA synthesis genome-wide. Of the 64 mouse replication-coupled genes, 54 are within the major histone gene cluster on Chromosome 13, and when Tumor and Normal dissection data from multiple experiments are displayed, differences are seen between tumor samples consistent with the observation of RNAPII hypertranscription differing between samples (FIG. 28F). For quantitative validation, the excess of normalized counts for each experiment were calculated, with strongly significant increases for the four RELA and three PDGFB-2 biological replicates and for PDGFB-1, with a small weakly significant increase for YAP1. The consistency between the measurements of hypertranscription over gene regulatory elements and S-phase-dependent hypertranscription over histone loci confirm that hypertranscription is a real, but variable tumor specific property of transgene-driven mouse tumors. As these exceptionally S-phase dependent histone loci are expressed in proportion to the amount of replicated DNA (23), we suggest that cancer cells increase engaged RNAPII at these loci to load up on histones at S phase for increased cell proliferation.

### Hypertranscription Varies Between Human Tumors

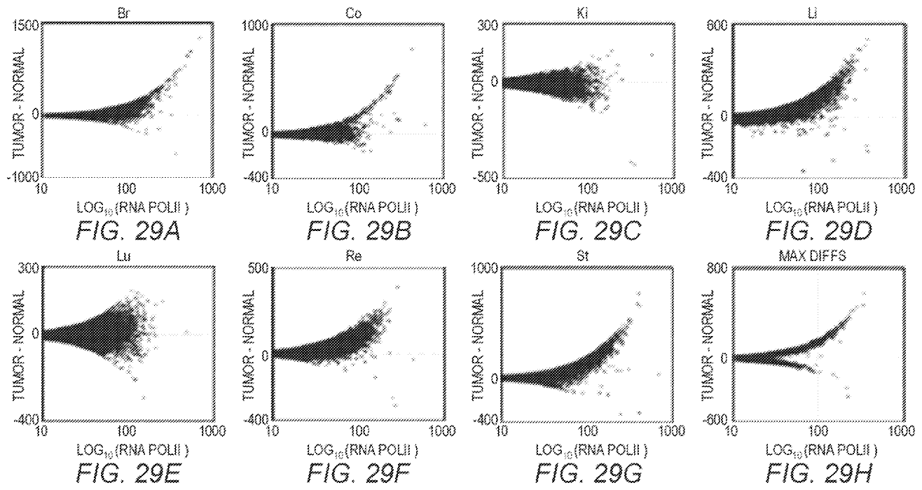

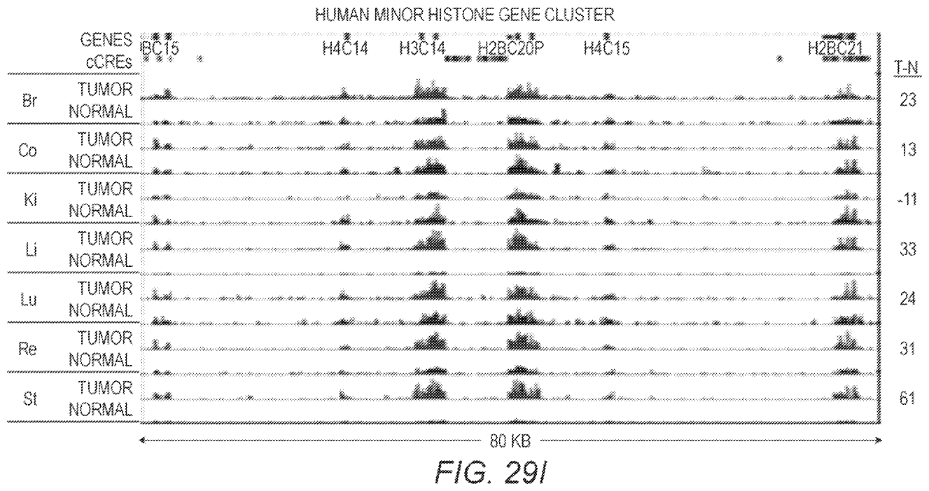

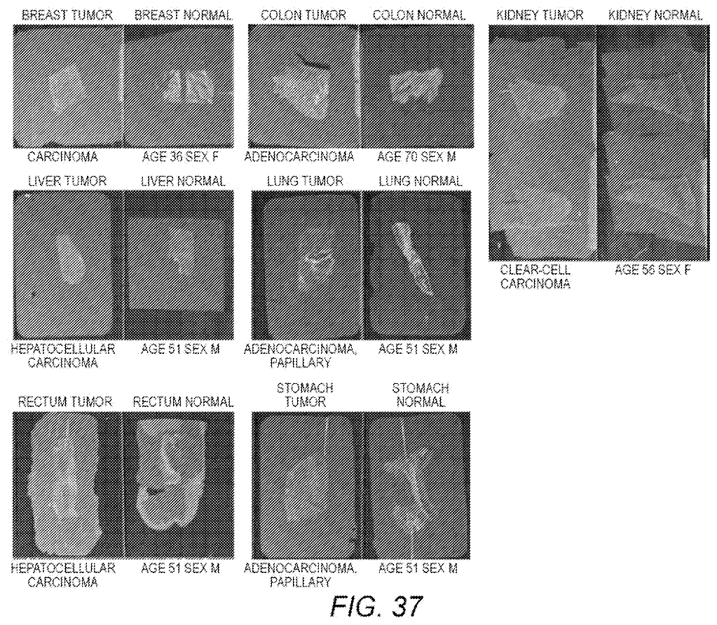

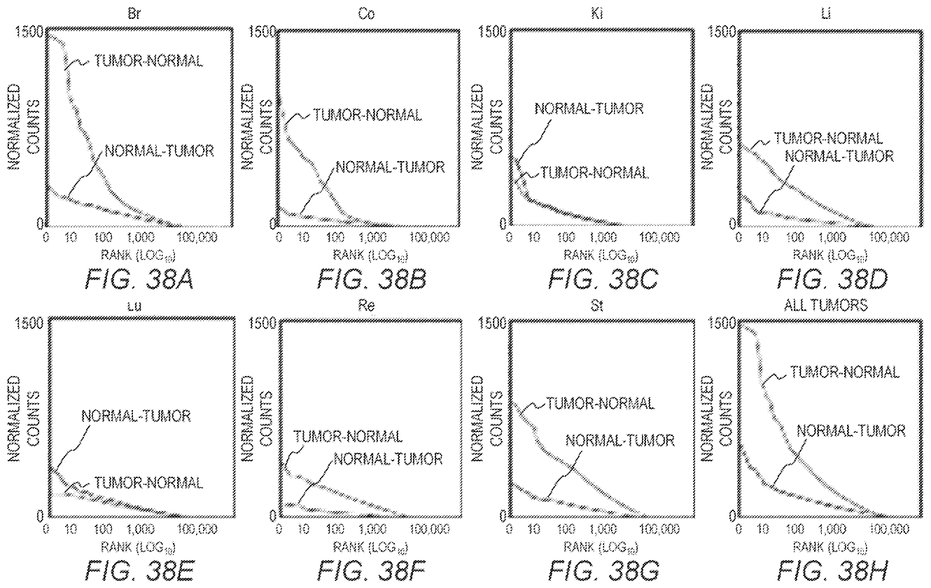

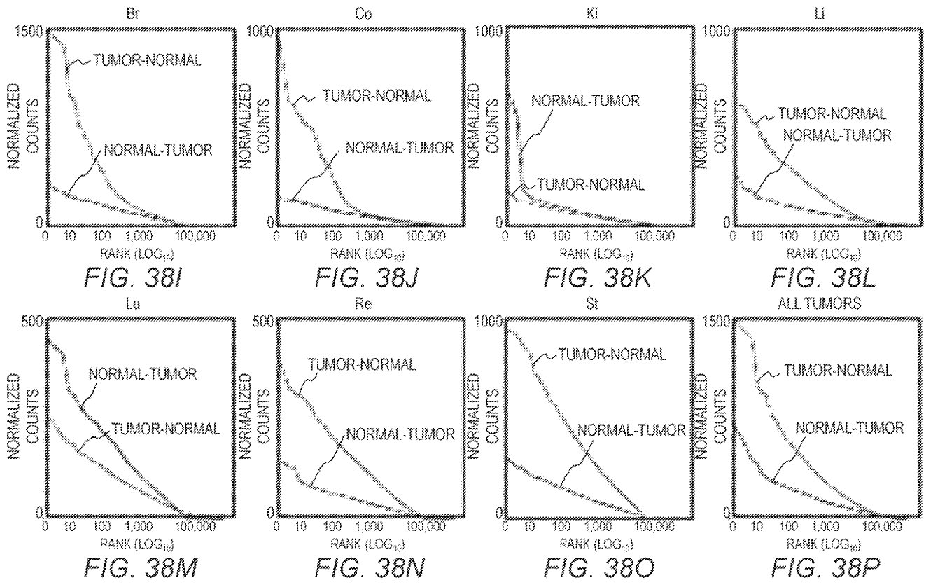

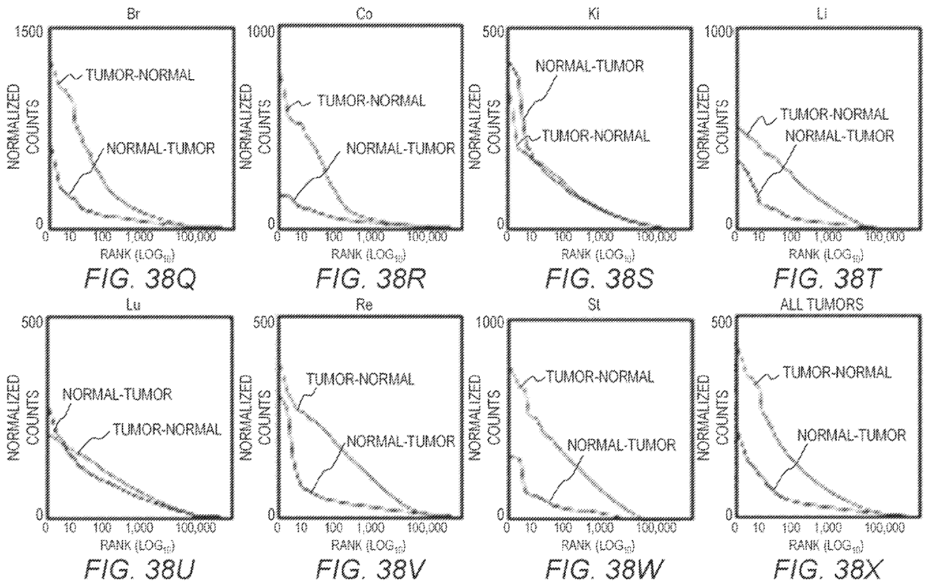

To expand on our findings of hypertranscription based on transgene-driven mouse brain tumors to a diverse sample of naturally occurring cancers, 5 μm FFPE sections on slides prepared from paraffin blocks of anonymous human tumor and adjacent normal pairs were obtained (FIG. 37). RNAPII-Ser5p FFPE-CUTAC was performed, and each pair rank-ordered by Tumor minus Normal differences to test for RNAPII hypertranscription based on the 984,834 ENCODE-annotated human cCREs. To avoid possible imbalances in the comparisons between tumor and normal pairs, cCREs in repeat-masked regions of the hg19 build were removed, the data pooled from all four independent experiments and equalized the number of fragments between tumor and normal samples. Clear hypertranscription in five of the seven samples (Br, Co, Li, Re and St) and for the composite of all samples were observed (FIGS. 29A-29H). In contrast, the Ki and Lu tumors showed essentially no hypertranscription. To evaluate the robustness of these hypertranscription results, hypertranscription for the data from a single slide for each specimen were plotted, and despite sparse data owing to the ~1 cm² size of the 5 μm sections very similar results were observed (FIGS. 38A-38P). Very similar results were also obtained when duplicates were removed and the number of fragments for each tumor-normal pair equalized (FIGS. 38Q-38X).

As was the case for the mouse tumors, hypertranscription could be observed by examining the human replication-coupled histone loci. Although the data were relatively sparse, the Br, Li, Re and St cancer samples showed prominent hypertranscription over the ~80-kb region spanning the human minor histone cluster whereas the Ki sample showed hypotranscription and the Lu sample showed little if any difference in RNAPII abundance (FIG. 291). Unexpectedly, our Co cancer sample showed no detectable difference between tumor and normal at the histone loci, despite the strong hypertranscription over cCREs. Together, the results suggest that FFPE-CUTAC can sensitively measure hypertranscription in small sections of the type that are routinely used by pathologists for cytological staining and analysis.

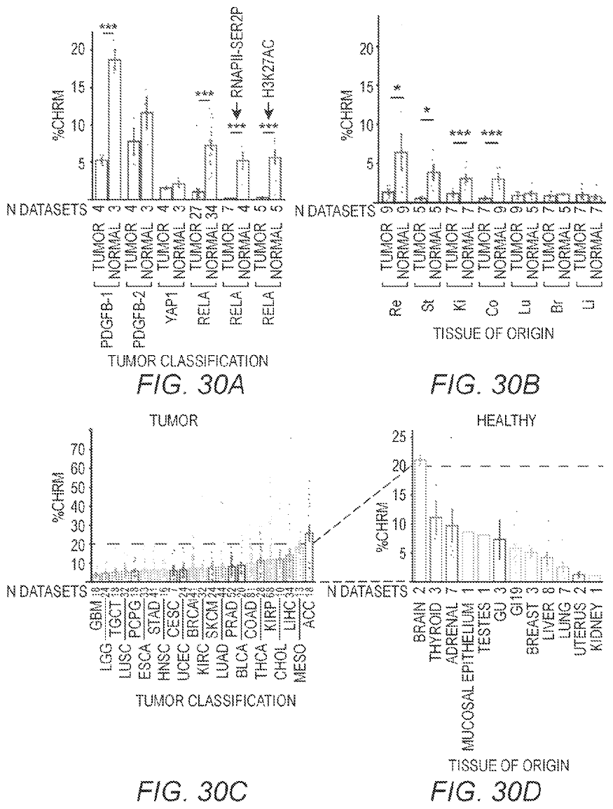

FFPE-CUTAC and other tagmentation methods non-specifically recover a small fraction of mitochondrial DNA (mtDNA, Chromosome M) due to the enhanced accessibility of nucleosome-free mtDNA. However, RNAPII-Ser5p FFPE-CUTAC detected a much lower level of mtDNA in most tumor samples than in their matched normal samples for both mouse and human (FIGS. 30A-30B), suggesting that these tumors contain fewer mitochondrial genomes. To test this interpretation, publicly available ATAC-seq data from both the TCGA and ENCODE projects were mined. In the case of TCGA tumor data, the percentage of mtDNA ranges from ~4% for glioblastoma, a brain cancer, ~25% for adrenal carcinoma, whereas for ENCODE data, which are from healthy individuals, percentages range from ~1% for kidney to ~21% for brain (FIGS. 30C-30D). This 6-fold higher level of mitochondrial ATAC-seq signal in normal brain in the ENCODE data over that of glioblastoma in the TCGA data is consistent with decreased mitochondrial DNA abundance in most human and mouse tumors in the FFPE-CUTAC data. These reductions in mtDNA by both CUTAC and ATAC-seq are consistent with reductions in mtDNA reported based on whole-genome sequencing (24), suggestive of relaxed selection for maintenance of mtDNA in cancer.

### Most Hypertranscribed Regulatory Elements are Shared Between Diverse Human Cancers

It was wondered whether the observations of hypertranscription in cancer based on annotated cCREs and histone genes could be generalized using an approach that does not depend on annotations of any kind. Previously, our lab introduced SEACR (Sparse Enrichment Analysis for CUT&RUN), which was designed for application to low read-count data (25). SEACR optionally uses a background control dataset, typically for a non-specific IgG antibody. To customize SEACR for hypertranscription in cancer, the background control was replaced with the normal sample in each pair, merged fragment data, duplicates removed and read numbers equalized for the seven human Tumor/Normal pairs. SEACR reported a median of 4483 peaks, and when Tumor and Normal were exchanged, a median of only 15 peaks was reported, which suggests that hypertranscription is more common than hypotranscription. Therefore, SEACR Tumor/Normal peaks can be used as an unbiased method for discovering the most hypertranscribed loci in the human cancer samples.

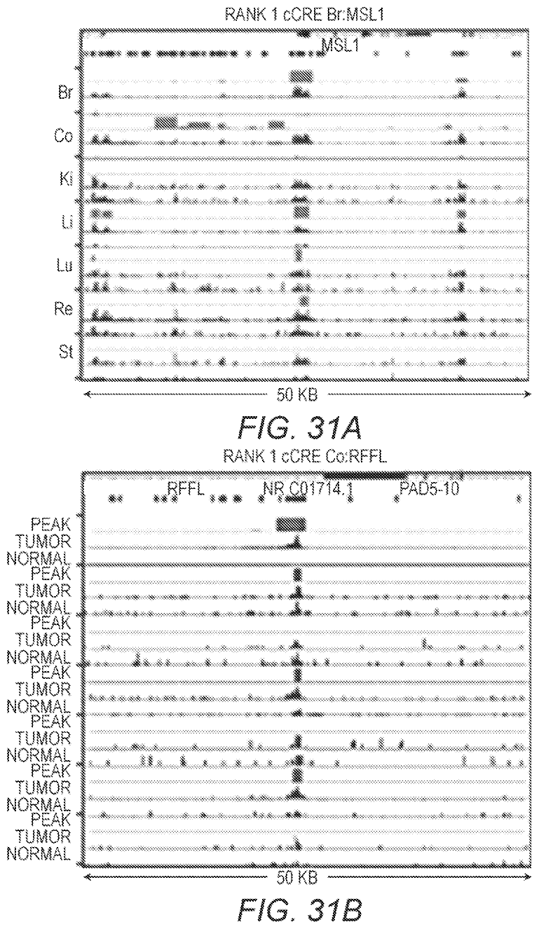

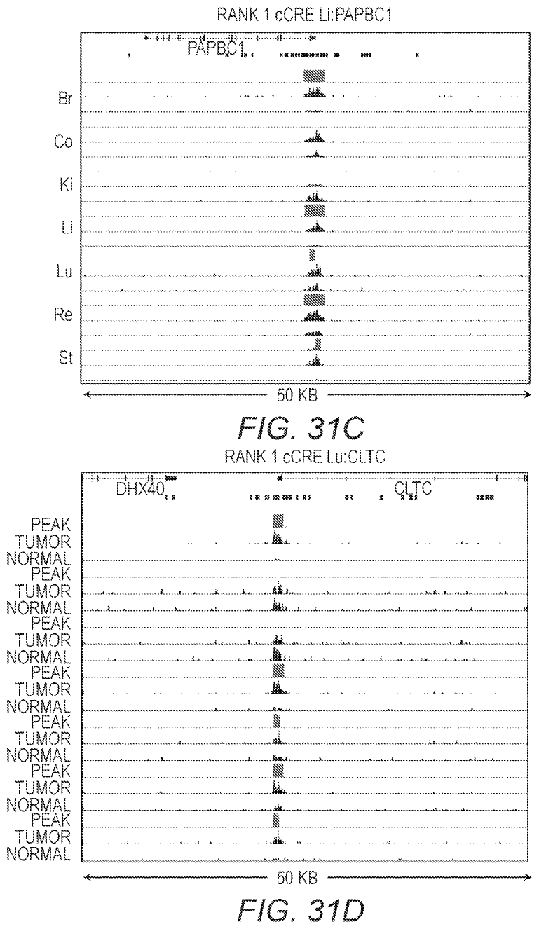

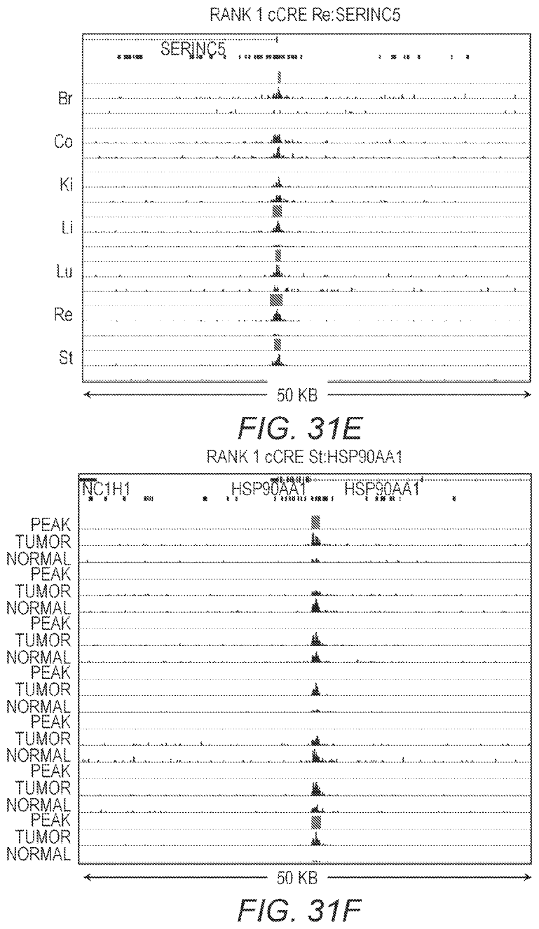

It was first asked whether SEACR Tumor/Normal peak calls corresponded to the 100 top-ranked cCREs in the overall list representing all seven tumors. Remarkably, all 100 cCREs at least partially overlapped one or more SEACR Tumor/Normal peak call, and in addition, the large majority of the 100 top-ranked cCREs intersected with overlapping SEACR peak calls from multiple Tumor/Normal pairs (Table 2). Each of the #1-ranked cCREs in the Br, Co, Li, Lu and Re tumor samples respectively intersected MSL1, RFFL, PABPC1, CLTC and SERINC5 genes and also overlapped SEACR peak calls in 4-5 of the 7 tumors (FIGS. 31A-31E). Additionally, the #1-ranked cCRE in the St sample intersected an intergenic enhancer in the HSP90AAl gene and overlapped SEACR peak calls in both Br and St (FIG. 31F). On average the same cCRE overlapped SEACR/Normal peak calls in 3.7 of the 7 tumors (Table 2). No SEACR peaks were observed for the kidney sample, as expected given the lack of detectable cCRE or histone locus hypertranscription. In conclusion, the large majority of strongly RNAPII-hypertranscribed regulatory elements are hypertranscribed in multiple human cancers.

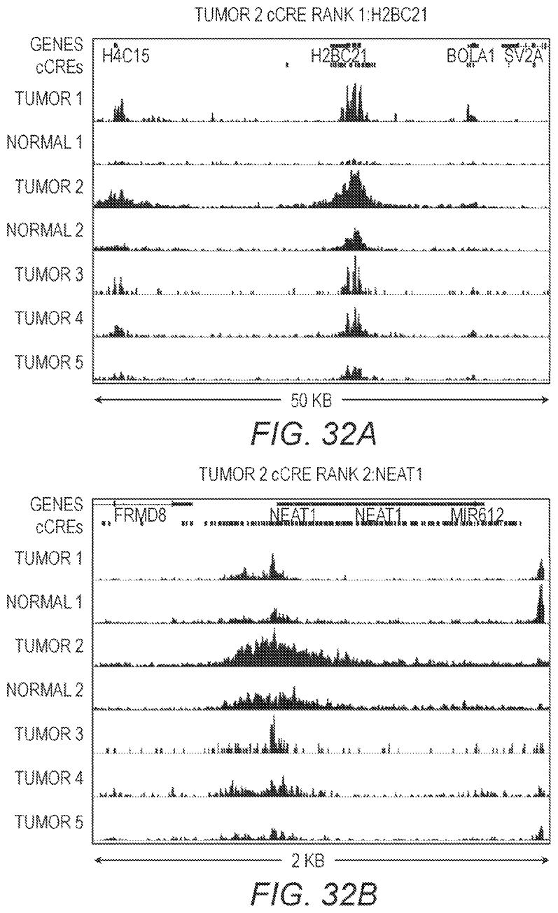

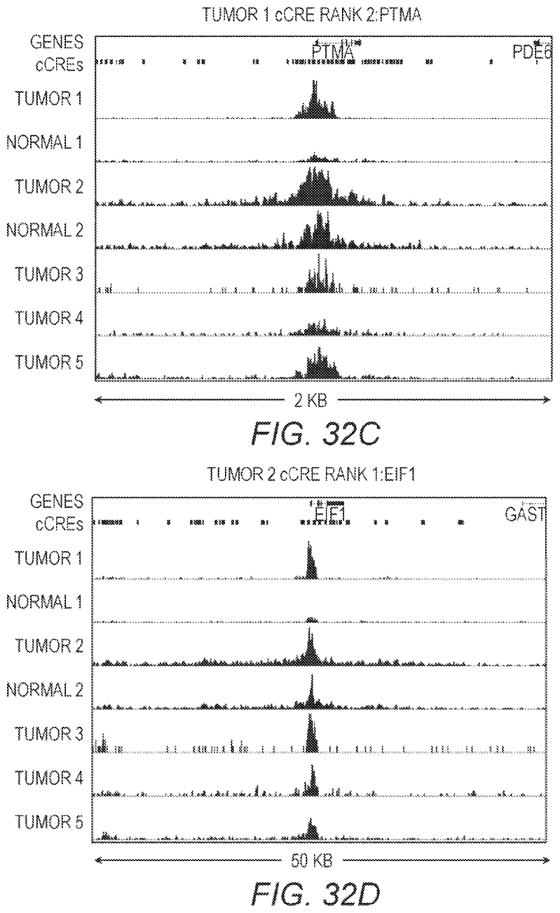

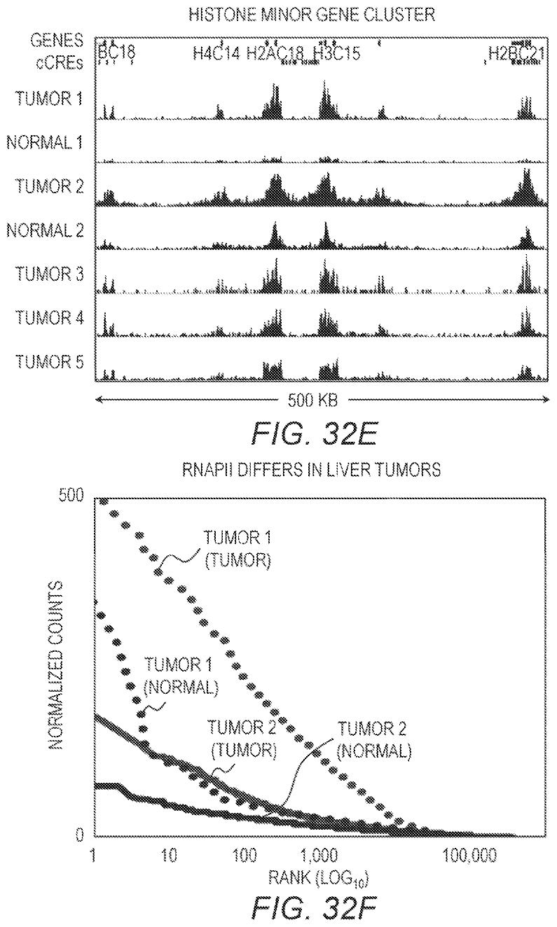

To test whether cell-type differences in hypertranscription can account for variability between human tumors in our sample, 10 μm FFPE tissue sections were obtained from a matched liver tumor/normal pair and three additional liver tumors, all from different patients. RNAPII-Ser5p FFPE-CUTAC revealed that the hypertranscription differences between liver tumors from unrelated individuals conspicuously differed. For example, all four cCREs that ranked #1 and #2 in either liver tumor showed strong hypertranscription in the first liver tumor but only weak hypertranscription in the second (FIGS. 32A-32D), and similar results were observed for the replication-coupled histone genes (FIG. 32E). Hypertranscription for the top-ranked >10,000 cCREs was observed for both liver tumor samples, again much stronger for the first tumor than for the second (FIG. 32F).

### Sparse FFPE-CUTAC Data Resolves Tumor Diversity

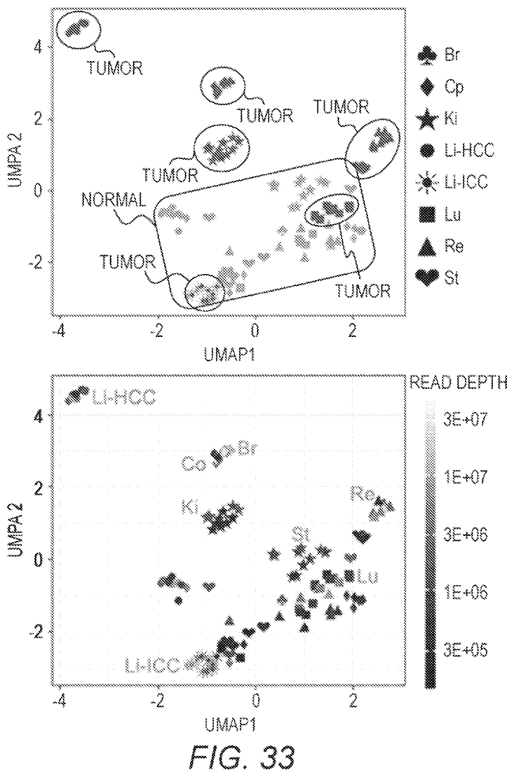

The identification of so many of the top hypertranscribed loci shared by multiple individual tumors raised the question of how effectively FFPE-CUTAC can resolve differences between cancers. Accordingly, a cCRE-based UMAP including all 114 individual human samples with >100,000 mapped fragments (median 925,820) was constructed. Whereas normal samples produced loose clusters with some mixing, tumor samples formed tight homogeneous clusters separated by cell type (FIG. 33A). This implies that paused RNAPII at regulatory elements is more discriminating between tumors than between the tissues that the tumors emerge from. Relatively few samples and shallow sequencing depths were needed for tight clustering; for example, the stomach tumor cluster comprised four samples from four different experiments with a median of ~470,000 mapped fragments (FIG. 33B). Interestingly, the Co and Br tumor samples clustered very close to one another, suggesting that this pair of individual tumors share oncogenic loci to a much greater extent than would be expected for such different tissue types.

HER2 Amplifications with Linkage Disequilibrium in Human Tumors

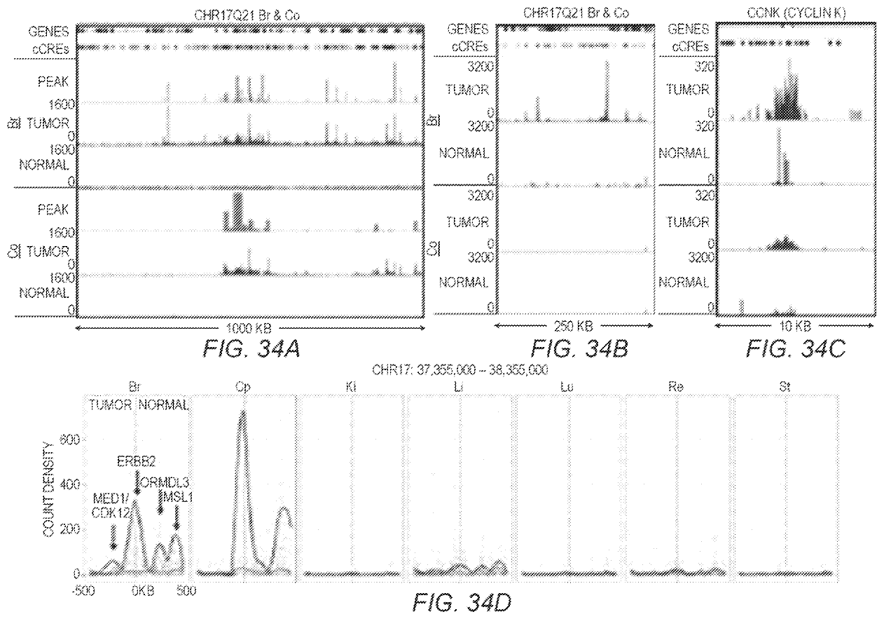

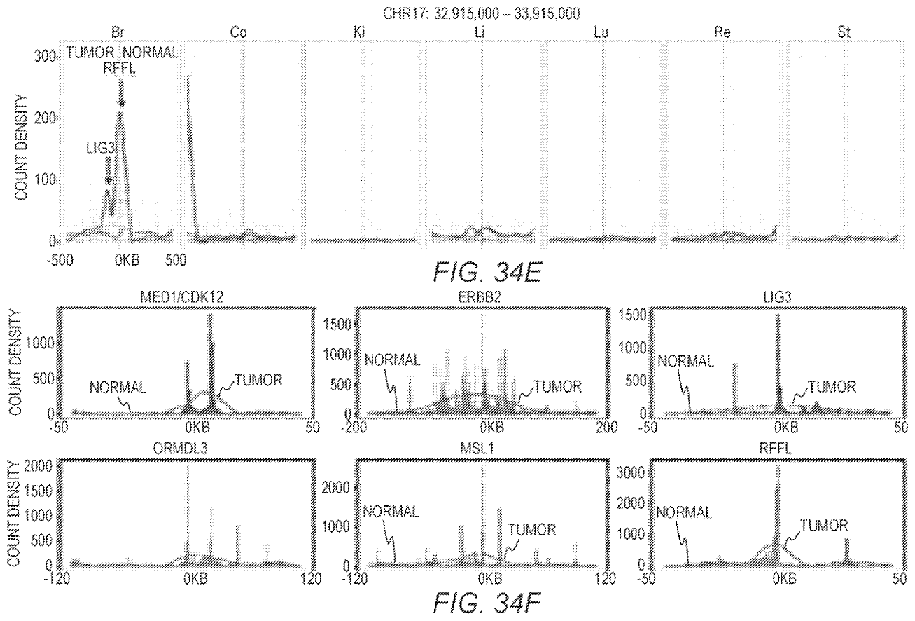

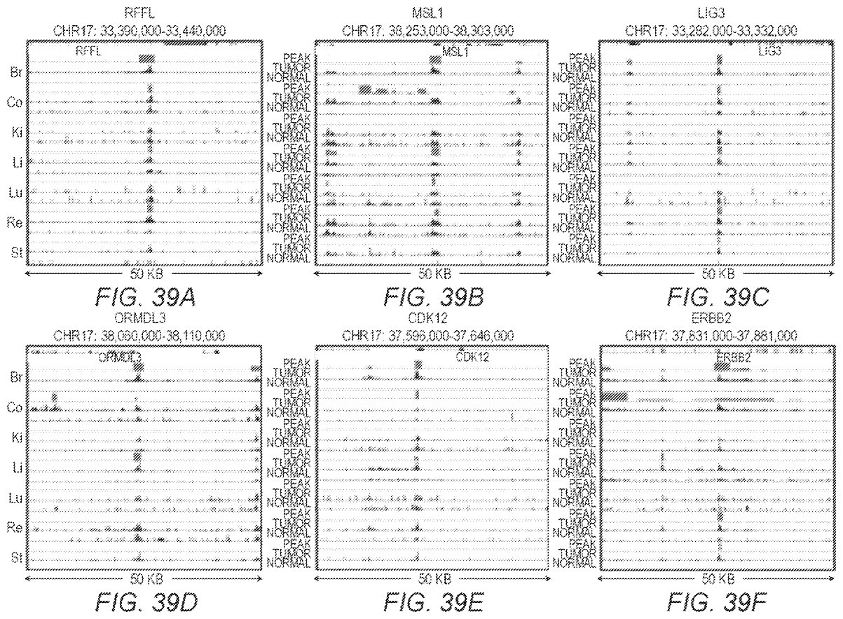

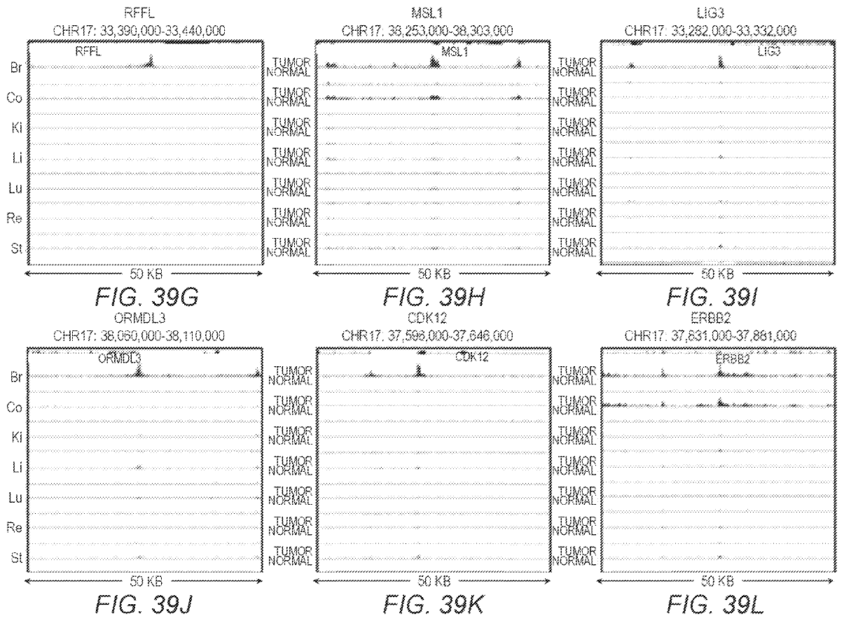

A clue to the basis for the close clustering of the breast and colon tumor samples was revealed in the list of the top 100 hypertranscribed cCREs: Eighteen of the top 20 cCREs are located on Chromosome 17 (FIGS. 39A-39F; Table 2). Most of these cCREs are within either of two contiguous RNAPII-Ser5p-enriched regions (Chr17q12 and Chr17q21) of a few hundred kilobases in length in the breast tumor sample not seen in the adjacent normal tissue (FIGS. 34A-34B). For the Co tumor sample a broad region of RNAPII enrichment is sharply defined within Chr17q21. The breadth of this region can explain why the Co sample was highly represented on Chromosome 17 based on the ranked cCRE list but showed no hypertranscription at the histone gene cluster. Major differences between the Br and Co tumors can be seen when sub-regions are group-autoscaled, which identified sharply defined promoter peaks just a few kilobases wide over RFFL, LIG3, ORMDL3 and CDK12 only in the Br tumor and MSL1 and ERBB2 in both the Br and Co tumor (FIGS. 39G-39L).

To identify the likely source of regional hypertranscription, PubMed was searched for each of the 100 top-ranked genes with the word “cancer”. This revealed that the most frequently named gene in titles and abstracts by far is ERBB2 in Chr17q21 (35,121 PubMed hits), which accounts for 2/3 of the total, where the next most frequently named gene in the same Chr17q21 region is CDK12 (413 PubMed hits) (Table 2). ERBB2 encodes Human Epidermal Growth Factor 2 Receptor (HER2), which is commonly amplified in breast and other tumors and is a target of therapy (26). As our measures of hypertranscription are scaled to the human genome sequence, amplification of a region will appear as a proportional increase in the level of RNAPII over the amplified region, so that one can interpret regional hypertranscription in both the Br and Co tumor samples as revealing independent amplification events.

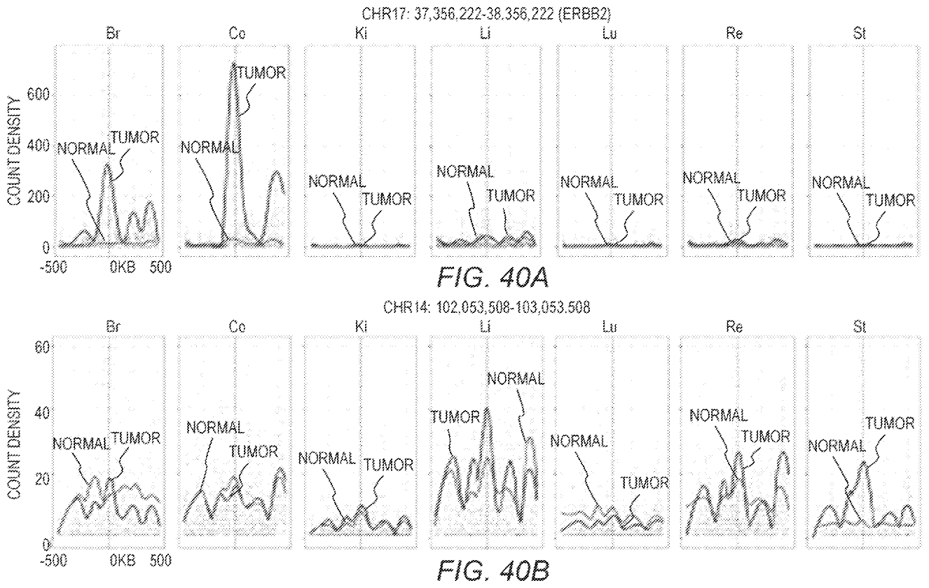

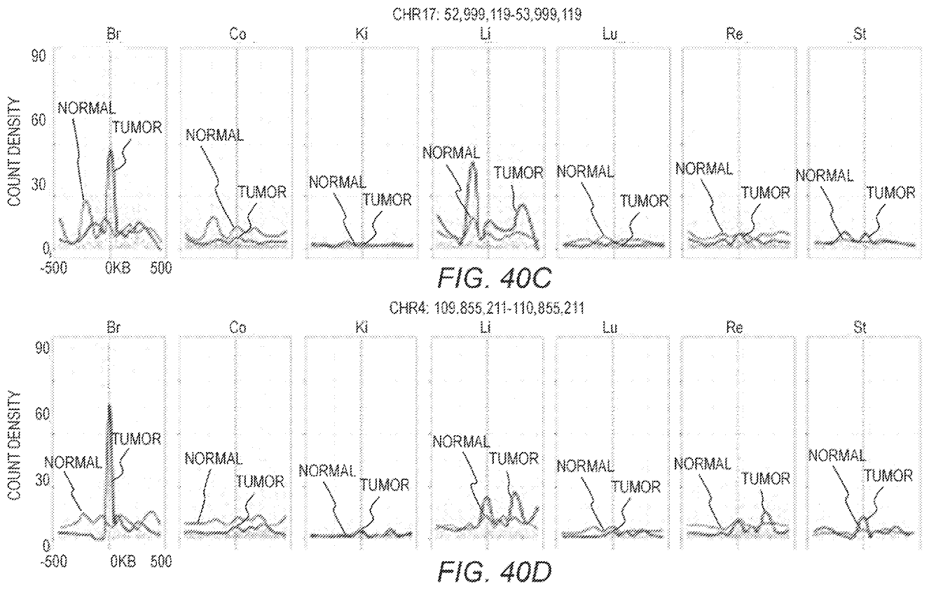

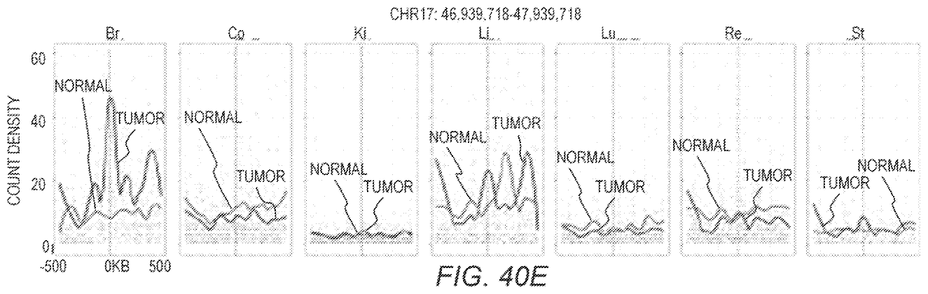

To delineate possible RNAPII hypertranscription features within Chr17q12 and Chr17q21, we tiled 1-kb bins over each 1 Mb region centered on the highest peak in Chr17q21, corresponding to the ERBB2 promoter in Chr17q21 and RFFL promoter in Chr17q12, and plotted count density within each bin with curve-fitting and smoothing. Remarkably, multiple broad summits appeared in both Br and Co tumor-versus-normal tracks (FIGS. 34D-34E), and the six summits in the Br tumor sample accounted for the six highest ranked Chr17 promoter peaks (FIGS. 39A-39F). We similarly plotted count densities of the four highest ranking cCREs outside of Chr17q12-21 (Table 2), but tumor peaks in these regions were at least an order-of-magnitude lower than the ERBB2 peaks in the Br and Co tumor samples (FIGS. 40A-40E). Of the six summits in the BR sample ERBB2 and MSL1 also appeared in the Co sample, whereas no other tumor samples showed prominent summits above normal in Chr17q12-21 (FIGS. 34D-34E). MSL1 encodes a subunit of a histone H4-lysine-16 acetyltransferase complex required for upregulation of the mammalian X chromosome (27).

Next, each of the six summits in the Chr17q12-21 region in the Br tumor sample were superimposed over the raw data tracks on expanded scales for clarity, centered over the highest promoter peak in the region (FIG. 34F). For ERBB2, the ~100 kb broad summit is almost precisely centered over the ~1 kb wide ERBB2 promoter peak. Although the other summits are less broad, each is similarly centered over a promoter peak. Insofar as there are multiple summits much broader than the promoter peaks that they are centered over, our results are inconsistent with independent upregulation of promoters over the HER2 amplified regions. Rather, it appears that a HER2 amplification event was followed by clonal selection for broad regions around ERBB2 and other loci within each amplicon.

Interestingly, one of the summits in the Br sample absent from Co sample corresponds to the bidirectional promoters of MED1 and CDK12, both of which have been shown to functionally cooperate with co-amplified ERBB2 in aggressive breast cancer (28, 29). MED1 encodes a subunit of the 26-subunit Mediator complex, which regulates RNAPII pause release, and CDK12 is the catalytic subunit of the CDK12/Cyclin K kinase heterodimer complex, which phosphorylates RNAPII on Serine-2 for productive transcriptional elongation. We wondered whether the co-amplification of these RNAPII regulators might directly drive hypertranscription throughout the genome. As Cyclin K is the regulatory subunit of the CDK12 kinase, onewould expect that the CCNK gene that encodes Cyclin K would be strongly upregulated in the Br tumor but not necessarily in the Co tumor. Indeed, we see a 5.4-fold increase in RNAPII-S5p over the CCNK promoter in the Br tumor relative to adjacent normal tissue, whereas in the Co tumor there is a 2.1-fold increase (FIG. 34C), consistent with RNAPII hypertranscription directly driven in part by CDK1 amplification.

### DISCUSSION

This example has shown that hypertranscription at gene regulatory elements can be measured directly with FFPE-CUTAC. Whereas hypertranscription in cancer had been frequently documented in studies based on RNA-seq (2, 3), these indirect methods have limitations owing to variable processing of mRNAs, to the low level of mRNAs encoding critical regulatory proteins and to the need for accurate calibration to genomic DNA abundance. Crucially, none of the methods that have been applied to measure hypertranscription in cancer are suitable for FFPEs, which have long been standard for archival storage of tissue samples (12). Exposure of tissue to ~4% formaldehyde for days badly damages RNA and DNA and causes cross-links to form between tightly bound proteins and nucleic acids. However, this formaldehyde treatment also forms covalent bonds between DNA and lysine-rich histones in nucleosomes rendering them inflexible, so that open chromatin gaps are the only accessible DNA in the nucleus. By using antibodies to the phosphorylated RNAPII heptapeptide repeat present in 52 lysine-free tandem copies or to the abundant histone H3K27ac mark of active regulatory elements, FFPE-CUTAC takes advantage of the hyperaccessibility and abundance of the targeted epitope and the impermeability of histone cross-linked chromatin to achieve exceptional signal-to-noise. As RNAPII FFPE-CUTAC maps the transcriptional machinery itself directly on the DNA regulatory elements, direct measurements of transcription initiation were obtained, as opposed to inferences based on estimating steady-state mRNA abundances. Thus, our mapping and quantitation of paused RNAPII, a critical checkpoint between transcriptional initiation and elongation, represents a powerful general approach to characterize hypertranscription at active regulatory elements genome-wide.

To quantify hypertranscription, we used normalized count differences between mouse tumor and normal tissue from the same FFPE sections and between matched human tumor and normal tissues, obviating the need for a spike-in normalization control. First, Tumor-Normal count differences were mapped for ENCODE-annotated cCREs, showing that nearly identical results were obtained regardless of whether the cCRE was a promoter, a proximal or distal enhancer or a CTCF (insulator) site. Second, hypertranscription within these samples was confirmed by examining replication-coupled histone clusters, which serve as proxies for cell proliferation. Third, a completely unsupervised approach was applied using our SEACR peak-caller to identify hypertranscribed loci throughout the genome. Remarkably, SEACR identified all of the 100 top-ranked of nearly 1 million human cCREs in at least one tumor (Table 2), reporting a median of 3.7 overlapping cCREs in six of the seven different human tumors in our study. Reductions in mitochondrial DNA that varied between tumors were also observed, suggestive of relaxed selection for mtDNA-encoded products during cancer progression. The rich regulatory information that can be extracted from RNAPII FFPE-CUTAC data using simple analytical tools, despite the use of sparse tissue samples in very poor condition relative to fresh or frozen samples, makes our method especially attractive for application of data mining tools that can be used to infer gene regulatory networks.

Finally, it was observed that 55 of the overall top-ranked 100 human cCREs mapped to extensive regions of hypertranscription within Chromosome 17q12-21 in the Br and Co cancer samples, characteristic of HER2 amplifications, which are especially common in breast and colorectal cancer (30). HER2 amplifications are known to be subject to clonal selection, resulting in tumor heterogeneity (31), consistent with our observation of broad summits centered directly over promoters of candidate cancer driver genes within the amplified regions. Thus, three of the four loci showing apparent linkage disequilibrium around ERBB2 in our breast and colon tumor samples are known or potential cancer drivers, consistent with the observation of clonally heterogeneous HER2 amplifications in primary breast tumors by whole-genome sequencing (31). Clonal selection may be driven by selective sweeps (32) following amplification events that generate extrachromosomal DNA in double-minute acentric chromosomes which partition unequally during each cell division (33). The evidence for linkage disequilibrium in the Br and Co samples that were analyzed suggests multiple selective sweeps resulting in loss of adjacent but physically unlinked DNA during evolution of these two tumors. Such copy number gains within a tumor can result in intra-tumor heterogeneity (17, 18) and are potential factors for resistance to therapy (34). FFPE-CUTAC thus potentially provides a general diagnostic strategy for detection and analysis of amplifications and clonal selection during cancer progression and therapeutic treatment.

Of the six broad summits observed in the breast tumor sample, those centered over ERBB2 and the bidirectional promoters of MED1 and CDK12 were already known to be associated with poor prognosis in HER2-positive breast cancer (28, 29), and MSL1 is part of the complex that upregulates the mammalian X-chromosome (27). Of particular interest is CDK12, a cyclin-dependent kinase that phosphorylates RNAPII on Serine-2 for pause release and transcriptional elongation and which is co-amplified with HER2 in ~90% of HER2+breast cancers (35). It was found that Cyclin K, the regulatory subunit of the CDK12/Cyclin K kinase complex is strongly upregulated in the same tumor, which suggests that amplification of CDK12 directly contributes to RNAPII hypertranscription and is in part responsible for poor prognosis in HER2/CDK12-amplified breast cancer patients (28, 35, 36). The application of FFPE-CUTAC to cohorts of HER2-amplified and other cancer patient samples is envisioned to ascertain the generality of the model for hypertranscription.

In summary, the high signal-to-noise and the abundance of RNAPII and H3K27ac epitopes used in FFPE-CUTAC have made possible detection of genome-wide hypertranscription using single 5 μm thick FFPE tissue sections ~1 cm² in area and fewer than 4 million unique fragments. Our identification of HER2 amplifications and probable clonal selection events that did not rely on reference to any external data emphasizes the potential power of our approach for understanding basic genetic and epigenetic mechanisms underlying tumor evolution. The simple workflow of FFPE-CUTAC and its potential for scale-up and automation make it an attractive platform for retrospective studies and will require little modification for routine cancer screening and other personalized medicine applications.

### Methods

### Mouse Tumor and Normal Tissues and FFPEs

Ntva;cdkn2a-/-mice were injected intracranially with DF1 cells infected with and producing RCAS vectors encoding either PDGFB (21), ZFTA-RELA (19), or YAP1-FAM118b (20) as has been described (37). Upon weaning (~P21), mice were housed with same-sex littermates, with no more than 5 per cage and given access to food/water ad libitum. When the mice became lethargic and showed poor grooming, they were euthanized and their brains removed and fixed at least 48 hours in neutral buffered formalin. All animal experiments were approved by and conducted in accordance with the Institutional Animal Care and Use Committee of Fred Hutchinson Cancer Center (Protocol #50842: Tva-derived transgenic mouse model for studying brain tumors). Tumorous and normal brains were sliced into five pieces and processed overnight in a tissue processor, mounted in a paraffin block and 10-micron sections were placed on slides. Mouse tissue (including normal and tumor bearing brains) were removed, fixed in 10% neutral-buffered formalin for a minimum of 24 hours and embedded into paraffin blocks. 10-μm serial sections were cut from formalin-fixed paraffin-embedded specimens and mounted on slides.

### Human FFPE Slides

The following pairs of human tumor and adjacent normal 5 μm tissue sections from single FFPE blocks were purchased from Biochain, Inc: Breast Normal/Tumor cat. no. T8235086PP/PT; Colon Normal/Tumor cat. no. T8235090PP/PT; Kidney Normal/Tumor cat. no. T8235142PP/PT; Liver Normal/Tumor cat. no. T8235149PP/PT; Lung Normal/Tumor cat. no. T8235152PP/PT; Rectum Normal/Tumor cat. no. T8235206PP/PT; Stomach Normal/Tumor cat. no. T8235248PP/PT. Human primary liver tumor and normal samples were harvested from cases undergoing surgical resection at the University of Washington under the Institutional Review Board approved protocol and then subsequently deidentified.

### Antibodies

Primary antibodies: RNAPII-Ser5p: Cell Signaling Technologies cat. no. 13523, lot 3; RNAPII-Ser2p: Cell Signaling Technologies cat. no. 13499; H3K27ac: Abcam cat. no. ab4729, lot no. 1033973. Secondary antibody: Guinea pig a-rabbit antibody (Antibodies online cat. no. ABIN101961, lot 46671).

### On-Slide FFPE-CUTAC

On-slide FFPE-CUTAC was performed as described (13) with modifications. Briefly, FFPE slides were placed in 800 mM Tris-HCl pH8.0 in a slide holder and incubated at 85-90° C. for 1-14 hours, whereupon the paraffin melted and floated off the slide. Slides were cooled to room temperature and transferred to 20 mM HEPES pH7.5,150 mM NaCl. Slides were drained and excess liquid wicked off using a Kimwipe tissue. The sections were immediately covered with 20-60 μL primary antibody in Triton®-Wash buffer (20 mM HEPES pH 7.5,150 mM NaCl, 2 mM spermidine and Roche complete EDTA-free protease inhibitor) added dropwise. Plastic film was laid on top to cover and slides were incubated ≥2 hr incubation at room temperature (or overnight at ~8° C.) in a moist chamber. The plastic film was peeled back, and the slide was rinsed once or twice by pipetting 1 mL Triton®-Wash buffer on the surface, draining at an angle. This incubation/wash cycle was repeated for the guinea pig anti-rabbit secondary antibody (Antibodies Online cat. no. ABIN101961) and for pAG-Tn5 preloaded with mosaic end adapters (Epicypher cat. no. 15-1117 1:20), followed by a Triton®-Wash rinse and transfer of the slide to 10 mM TAPS pH 8.5. Tagmentation was performed in 5 mM MgCl₂, 10 mM TAPS pH 8.5, 20% (v/v) N,N-dimethylformamide in a moist chamber and incubated at 55° C. for 1 hr. Following tagmentation, slides were dipped in 10 mM TAPS pH 8.5, drained and excess liquid wicked off. Individual sections were covered with 2 μL 10% Thermolabile Proteinase K (TL ProtK) in 1% SDS using a pipette tip to loosen the tissue. Tissue was transferred to a thin-wall PCR tube containing 2 μL TL ProK using a watchmaker's forceps, followed by 1 μL TL ProtK and transfer to the PCR tube. Tubes were incubated at 37° C. for 30 min and 58° C. for 30 min before PCR as described above.

### FFPE-CUTAC for Curls

Curls were transferred to a 1.7 mL low-bind tube (Axygen cat. no. MCT-175-C), which tightly fits a blue pestle (Fisher cat. on. 12-141-364). Mineral oil (200 μl) was added and the tube was placed in a 85-90° C. water bath for up to 5 min to melt the paraffin. The suspension was then homogenized ~10-20 sec with a pestle attached to a pestle motor (DWK Life Sciences cat no. 749540-0000). Warm cross-link reversal buffer (200 μl 800 mM Tris-HCl pH8.0) was added followed by addition of 6 μl of 1:10 Biomag amine paramagnetic beads (48 mg/ml, Polysciences cat. no. 86001-10). Homogenization was repeated, and 800 μl warm cross-link reversal buffer was added. Tubes were incubated at 85-90° C. for 1-14 hours, vortexed, centrifuged briefly and the mineral oil was removed from the top without disturbing the surface. A 500 μl volume of mineral oil was added, mixed by inversion, centrifuged and the mineral oil removed leaving a thin oil layer. A 2.4 μl volume of agarose glutathione paramagnetic beads (Fisher cat. no. 88822) was added below the surface and mixed by inversion on a rotator. Tubes were centrifuged briefly, placed on a strong magnet (Miltenyi Macsimag separator, cat. no. 130-092-168), and the supernatant removed and discarded, and the bead-bound homogenate was resuspended in up to 1 mL Triton®-wash buffer (20 mM HEPES pH 7.5, 150 mM NaCl, 0.5 mM spermidine, 0.2 mM EDTA, 0.05% Triton®-X100 and Roche EDTA-free protease inhibitor) and divided into PCR tubes for antibody addition. Other steps through to library preparation and purification followed the standard FFPE-CUTAC protocol (13). Detailed step-by-step protocols for both slides and curls are available on Protocols.io: protocols.io/edit/cutac-for-ffpes-c5huy36w.

### DNA Sequencing and Data Processing

The size distributions and molar concentration of libraries were determined using an Agilent 4200 TapeStation. Barcoded CUT&Tag libraries were pooled at equal volumes within groups or at approximately equimolar concentration for sequencing. Paired-end 50×50 bp sequencing on the Illumina NextSeq 2000 platform was performed by the Fred Hutchinson Cancer Research Center Genomics Shared Resources.

### Data Analysis

### Preparation of the CCREs

We obtained the mm 10 and hg38 versions of the Candidate cis-Regulatory Elements by ENCODE (screen. encodeproject.org/) from UCSC (38). For mouse mm 10 we used all 343,731 entries. Because the sequencing data was aligned to hg19, we used UCSC's liftOver tool to re-position the hg38 CCREs resulting in 924,834 entries. It was noticed that many human CCREs were in repeated regions of the genome so the hg19 CCRE file was intersected with UCSC's RepeatMasked regions using bedtools 2.30.0 (39) “intersect-v” command to make a file of 464,749 CCREs not in repeated regions.

### Preparation of Histone Regions

For mm10 these regions were used:

| | | | |
|---|---|---|---|
| chr13 | 21715711 | 21837530 | H2bc13-H4bc2 |
| chr13 | 22035122 | 22043658 | H2ac12-H2bc11 |
| chr13 | 23531044 | 23622558 | H4c8-H1f4 |
| chr13 | 23683473 | 23764412 | H2ac6-H1f1 |

For hg19 these regions were used:

| | | | |
|---|---|---|---|
| chr1 | 149783434 | 149859466 | Minor |
| chr6 | 26017260 | 26285727 | Major |

Alignment of PE50 Illumina sequencing

1. Cutadapt 2.9 (40) was used with parameters “-j 8--nextseq-trim 20-m 20-a

(SEQ ID NO:)
AGATCGGAAGAGCACACGTCTGAACTCCAGTCA-A
(SEQ ID NO:)
AGATCGGAAGAGCGTCGTGTAGGGAAAGAGTGT-Z″

to trim adapters from 50 bp paired-end reads fastq files.

2. Bowtie2 2.4.4 (41) was used with options”--very-sensitive-local--soft-clipped-unmapped-tlen--dovetail--no-mixed--no-discordant-q--phred33-I 10-X 1000″ to map the paired-end 50 bp reads to the mm 10 Mus musculus or hg19 Homo sapiens reference sequences obtained from UCSC.

3. Samtools 1.14 (42) “view” was used to extract properly paired reads from the mm 10 alignments into bed files of mapped fragments.

4. The fraction of fragments mapped to chrM was computed.

5. Bedtools 2.30.0 “genomecov” was used to make a normalized count track which is the fraction of counts at each base pair scaled by the size of the reference sequence so that if the counts were uniformly distributed across the reference sequence there would be one at each position.

6. Picard 2.18.29 MarkDuplicates program (broadinstitute.github.io/picard/) was run on the sam output from bowtie2.

### Preparation of Aligned Samples

1. For mouse, all mapped fragments were used. For human, duplicates as marked by Picard were removed from the sam files before making normalized count tracks.

2. For mouse on-slide experiments, tumor replicates were merged within the experiment. For human, mapped fragments were merged from 5 different experiments for each tumor and then the numbers of fragments equalized for tumor and normal pairs by downsampling the larger of the two using the UNIX shuf command.

### Peak-Finding

SEACR 1.3 (25) was run with parameters “norm relaxed” on tumor samples with the normal sample from each tumor and normal pair as the control. For comparison, we also called peaks after reversing the roles of tumor and normal.

### Preparation of the Per-cCRE and Per-Histone Region Files

We used the bedtools intersect and groupby commands to sum the number of normalized counts from the tracks within the cCRE and histone region boundaries. Because the cCREs and histone regions vary in size, we then averaged the number of normalized counts within each to make them more comparable. The resulting files have one row per cCRE or histone region and one column per sample and are suitable for submission to the Degust server (degust.erc.monash.edu/) using the Voom/Limma option (-log 10FDR versus log 2FoldChange).

Preparation of Tumor-Normal files

Tumor-Normal pairs were computed from the CCRE region files and sorted by largest differences in absolute value (Table 2).

### Curve-Fitting

The genome was partitioned into 1 kb tiles and merged replicates, then downsampled to equalize library sizes between tumor and normal samples from each patient and normalized counts within each tile added up. For each tumor and normal patient sample, the normalized counts were fit across tiles using a Local Polynomial Regression (LOESS) model as implemented in the ‘stats’ package of the R programming language, setting the degree of smoothing to 0.2 (FIGS. 34C-34D) or 0.5 (FIG. 34E) specified by the ‘span parameter of ‘loess function.

### UMAPs

To ensure the quality of samples for downstream analysis, we excluded samples with fewer than 100,000 read counts or less than 10,000,000 bp of total fragment length. We utilized cCRE regions as the genomic features and calculated the raw sequencing read count overlapping each cCRE region using the “getCounts” function from the chrom VAR R package. Processing the cCRE regions by samples count matrix, we initially applied the term frequency-inverse document frequency (TF-IDF) normalization method (43). This method first normalizes read counts across samples to correct for differences in total read depth and then adjusts across cCRE regions, assigning higher values to rarer regions. TF-IDF normalization was implemented using the “RunTFIDF” function from the Signac R package. This step was followed by the selection of top features using “FindTopFeatures” from the Signac package and data scaling performed by “ScaleData” from the Seurat package. Subsequently, we conducted principal component analysis (PCA) on the scaled data and then used the top 50 principal components to generate a UMAP representation, providing a refined visualization of the relationship across samples.

### Statistics and Reproducibility

No statistical method was used to predetermine sample size nor were data excluded from the analyses. The experiments were not randomized and Investigators were not blinded to allocation during experiments and outcome assessment.

### Data Availability

The sequencing data generated in this study have been deposited in the NCBI GEO database under accession code GSE261351.

### Code Availability

Custom scripts used in this study are available from GitHub: github.com/Henikoff/FFPE.

### REFERENCES

1. Percharde M, Bulut-Karslioglu A, Ramalho-Santos M. Hypertranscription in Development, Stem Cells, and Regeneration. Dev Cell. 2017; 40 (1): 9-21.
2. Zatzman M, Fuligni F, Ripsman R, Suwal T, Comitani F, Edward L M, et al. Widespread hypertranscription in aggressive human cancers. Sci Adv. 2022; 8 (47): eabn0238.
3. Cao S, Wang JR, Ji S, Yang P, Dai Y, Guo S, et al. Estimation of tumor cell total mRNA expression in 15 cancer types predicts disease progression. Nat Biotechnol. 2022; 40 (11): 1624-33.
4. Nie Z, Hu G, Wei G, Cui K, Yamane A, Resch W, et al. c-Myc is a universal amplifier of expressed genes in lymphocytes and embryonic stem cells. Cell. 2012; 151 (1): 68-79.
5. Lin C Y, Loven J, Rahl P B, Paranal R M, Burge C B, Bradner J E, et al. Transcriptional amplification in tumor cells with elevated c-Myc. Cell. 2012; 151 (1): 56-67.
6. Loven J, Orlando D A, Sigova A A, Lin C Y, Rahl P B, Burge C B, et al. Revisiting global gene expression analysis. Cell. 2012; 151 (3): 476-82.
7. Patange S, Ball D A, Wan Y, Karpova T S, Girvan M, Levens D, et al. MYC amplifies gene expression through global changes in transcription factor dynamics. Cell Rep. 2022; 38 (4): 110292.
8. Henikoff S, Henikoff J G, Ahmad K, Paranal R M, Janssens D H, Russell Z R, et al. Epigenomic analysis of formalin-fixed paraffin-embedded samples by CUT&Tag. Nat Commun. 2023; 14 (1): 5930.
9. Lau M S, Hu Z, Zhao X, Tan Y S, Liu J, Huang H, et al. Transcriptional repression by a secondary DNA binding surface of DNA topoisomerase I safeguards against hypertranscription. Nat Commun. 2023; 14 (1): 6464.
10. Kim Y K, Cho B, Cook D P, Trcka D, Wrana J L, Ramalho-Santos M. Absolute scaling of single-cell transcriptomes identifies pervasive hypertranscription in adult stem and progenitor cells. Cell Rep. 2023; 42 (1): 111978.
11. Network TCGA. Comprehensive molecular portraits of human breast tumours. Nature. 2012; 490 (7418): 61-70.
12. Blow N. Tissue preparation: Tissue issues. Nature. 2007; 448 (7156): 959-63.
13. Henikoff S, Henikoff J G, Ahmad K, Paranal R M, Janssens D H, Russell Z R, et al. Epigenomic analysis of Formalin-fixed paraffin-embedded samples by CUT&Tag. Nat Commun. 2023; 14:5930.
14. Henikoff S, Henikoff J G, Kaya-Okur H S, Ahmad K. Efficient chromatin accessibility mapping in situ by nucleosome-tethered tagmentation. eLife. 2020; 9: e63274.
15. Janssens D H, Otto D J, Meers M P, Setty M, Ahmad K, Henikoff S. CUT&Tag2 for1: a modified method for simultaneous profiling of the accessible and silenced regulome in single cells. Genome Biol. 2022; 23 (1): 81.
16. Henikoff S, Henikoff J G, Ahmad K. Simplified Epigenome Profiling Using Antibody-tethered Tagmentation. bio-protocol. 2021; 11 (11): e4043.
17. Black J R M, McGranahan N. Genetic and non-genetic clonal diversity in cancer evolution. Nat Rev Cancer. 2021; 21 (6): 379-92.
18. Nowell P C. The clonal evolution of tumor cell populations. Science. 1976;194 (4260): 23-8.
19. Ozawa T, Arora S, Szulzewsky F, Juric-Sekhar G, Miyajima Y, Bolouri H, et al. A De Novo Mouse Model of C11orf95-RELA Fusion-Driven Ependymoma Identifies Driver Functions in Addition to NF-kappaB. Cell Rep. 2018; 23 (13): 3787-97.
20. Szulzewsky F, Arora S, Hoellerbauer P, King C, Nathan E, Chan M, et al. Comparison of tumor-associated YAPI fusions identifies a recurrent set of functions critical for oncogenesis. Genes Dev. 2020; 34 (15-16): 1051-64.
21. Dai C, Celestino JC, Okada Y, Louis DN, Fuller GN, Holland EC. PDGF autocrine stimulation dedifferentiates cultured astrocytes and induces oligodendrogliomas and oligoastrocytomas from neural progenitors and astrocytes in vivo. Genes Dev. 2001; 15 (15): 1913-25.
22. Altman DG, Bland JM. Measurement in medicine: the analysis of method comparison studies. The Statistician. 1983; 32:307-17.
23. Lu F, Park BJ, Fujiwara R, Wilusz JE, Gilmour D S, Lehmann R, et al. Integrator-mediated clustering of poised RNA polymerase II synchronizes histone transcription. bioRxiv. 2024.
24 Reznik E, Miller M L, Senbabaoglu Y, Riaz N, Sarungbam J, Tickoo S K, et al. Mitochondrial DNA copy number variation across human cancers. eLife. 2016; 5.
25. Meers M P, Tenenbaum D, Henikoff S. Peak calling by Sparse Enrichment Analysis for CUT&RUN chromatin profiling. Epigenetics Chromatin. 2019; 12 (1): 42.
26 Zhang H, Finkelman B S, Ettel M G, Velez M J, Turner B M, Hicks D G. HER2 evaluation for clinical decision making in human solid tumours: pearls and pitfalls. Histopathology. 2024; doi.org/10.1111/his.15170.
27. Deng X, Berletch J B, Ma W, Nguyen D K, Hiatt J B, Noble W S, et al. Mammalian X upregulation is associated with enhanced transcription initiation, RNA half-life, and MOF-mediated H4K16 acetylation. Dev Cell. 2013; 25 (1): 55-68.
28. Forster-Sack M, Zoche M, Pestalozzi B, Witzel I, Schwarz E I, Herzig J J, et al. ERBB2-amplified lobular breast carcinoma exhibits concomitant CDK12 co-amplification associated with poor prognostic features. The journal of pathology Clinical research. 2024; 10 (2): e12362.
29. Marotta M, Onodera T, Johnson J, Budd G T, Watanabe T, Cui X, et al. Palindromic amplification of the ERBB2 oncogene in primary HER2-positive breast tumors. Sci Rep. 2017; 7:41921.
30. Tanaka H, Watanabe T. Mechanisms Underlying Recurrent Genomic Amplification in Human Cancers. Trends in cancer. 2020; 6 (6): 462-77.
31. Fan Y, Zou L, Zhong X, Wang Z, Wang Y, Luo C, et al. Characteristics of DNA macro-alterations in breast cancer with liver metastasis before treatment. BMC Genomics. 2023; 24 (1): 391.
32. Yang D, Jones M G, Naranjo S, Rideout W M, 3rd, Min K HJ, Ho R, et al. Lineage tracing reveals the phylodynamics, plasticity, and paths of tumor evolution. Cell. 2022; 185 (11): 1905-23 e25.
33. Yan X, Mischel P, Chang H. Extrachromosomal DNA in cancer. Nat Rev Cancer. 2024; 24 (4): 261-73.
34. Schaff D L, Fasse A J, White P E, Vander Velde R J, Shaffer S M. Clonal differences underlie variable responses to sequential and prolonged treatment. Cell systems. 2024; 15 (3): 213-26 e9.
35. Choi H J, Jin S, Cho H, Won H Y, An H W, Jeong G Y, et al. CDK12 drives breast tumor initiation and trastuzumab resistance via WNT and IRS1-ErbB-PI3K signaling. EMBO Rep. 2019; 20 (10): e48058.
36. Wang Z, Himanen S V, Haikala H M, Friedel C C, Vihervaara A, Barboric M. Inhibition of CDK12 elevates cancer cell dependence on P-TEFb by stimulation of RNA polymerase II pause release. Nucleic Acids Res. 2023; 51 (20): 10970-91.
37. Hambardzumyan D, Amankulor N M, Helmy K Y, Becher O J, Holland E C. Modeling Adult Gliomas Using RCAS/t-va Technology. Translational oncology. 2009; 2 (2): 89-95.
38. Hinrichs A S, Karolchik D, Baertsch R, Barber G P, Bejerano G, Clawson H, et al. The UCSC Genome Browser Database: update 2006. Nucleic Acids Res. 2006; 34 (Database issue): D590-8.
39. Quinlan A R. BEDTools: The Swiss-Army Tool for Genome Feature Analysis. Current protocols in bioinformatics. 2014; 47:11 2 1-34.
40. Martin M. Cutadapt Removes Adapter Sequences From High-Throughput Sequencing Reads. EMBnetjournal. 2010; 17 DOI: 10.14806/ej.17.1.200.
41. Langmead B, Salzberg S L. Fast gapped-read alignment with Bowtie 2. Nat Methods. 2012; 9 (4): 357-9.
42. Danecek P, Bonfield J K, Liddle J, Marshall J, Ohan V, Pollard M O, et al. Twelve years of SAMtools and BCFtools. Gigascience. 2021; 10 (2).
43. Stuart T, Butler A, Hoffman P, Hafemeister C, Papalexi E, Mauck W M, 3rd, et al. Comprehensive Integration of Single-Cell Data. Cell. 2019; 177 (7): 1888-902 e21.

TABLE 2

| Rank | cCRE | Chr | start | end | Breast | Colon | Kidney | Liver | Lung | Rectum |
|---|---|---|---|---|---|---|---|---|---|---|
| 1 | EH38E1858185-enhP | chr17 | 33415789 | 33416133 | 2069.971 | 3.77 | −5.084 | 43.27 | 2.974 | 60.216 |
| 2 | EH38E1858186-prom | chr17 | 33416234 | 33416573 | 1815.186 | 28.839 | −0.76 | 16.05 | −2.218 | 63.912 |
| 3 | EH38E1861479-enhP | chr17 | 38278062 | 38278411 | 1353.096 | 450.344 | −16.95 | 116.3 | 31.333 | 23.238 |
| 4 | EH38E1858086-enhP | chr17 | 33307713 | 33307881 | 1261.804 | −0.511 | −53.17 | 178.1 | −26.74 | 14.42 |
| 5 | EH38E1861480-prom | chr17 | 38278421 | 38278683 | 1221.496 | 432.804 | 27.993 | 85.42 | 69.7 | 7.748 |
| 6 | EH38E1861258-prom | chr17 | 38083719 | 38084065 | 1049.48 | 2.88 | 32.392 | 212.8 | 36.083 | 17.217 |
| 7 | EH38E1743727-prom | chr14 | 1.03E+08 | 1.03E+08 | 208.443 | −127 | 65.468 | 341.2 | 96.15 | 239.32 |
| 8 | EH38E1858184-enhP | chr17 | 33415450 | 33415681 | 1039.974 | 10.386 | −0.408 | 43.19 | 9.696 | 84.664 |
| 9 | EH38E1860842-prom | chr17 | 37617967 | 37618285 | 1027.798 | 15.734 | −9.374 | 63.06 | −8.323 | 32.56 |
| 10 | EH38E1861478-enhP | chr17 | 38277719 | 38277885 | 983.622 | 360.317 | −10.41 | 38.35 | 48.123 | 16.607 |
| 11 | EH38E1871878-enhP | chr17 | 53499038 | 53499201 | 965.776 | 29.889 | −64.88 | 47.47 | 16.23 | 39.476 |
| 12 | EH38E1861257-enhP | chr17 | 38083486 | 38083677 | 964.408 | −0.2 | 5.321 | 170.7 | 0.158 | −22.337 |
| 13 | EH38E1861496-prom | chr17 | 38296481 | 38296831 | 949.349 | 484.696 | −25.72 | 181.2 | −23.27 | 39.212 |
| 14 | EH38E2319391-enhP | chr4 | 1.1E+08 | 1.1E+08 | 899.533 | 1.078 | 45.314 | 65.53 | −14.1 | 62.677 |
| 15 | EH38E1871879-prom | chr17 | 53499269 | 53499619 | 892.606 | 6.759 | 13.353 | 81.51 | −11.54 | 32.569 |
| 16 | EH38E1861482-enhP | chr17 | 38278994 | 38279161 | 869.45 | 525.449 | −31.66 | 63.54 | 2.152 | 30.349 |
| 17 | EH38E1868863-prom | chr17 | 47439545 | 47439892 | 849.809 | 16.766 | 42.223 | 102.2 | 22.221 | 20.366 |
| 18 | EH38E1861058-prom | chr17 | 37856088 | 37856357 | 597.824 | 815.99 | −37.51 | −2.83 | 12.354 | 12.464 |
| 19 | EH38E1861059-enhP | chr17 | 37856587 | 37856787 | 808.318 | 786.135 | −61.65 | 45.08 | 25.624 | 75.906 |
| 20 | EH38E1868862-prom | chr17 | 47439373 | 47439545 | 801.658 | 41.369 | 21.669 | 89.34 | 28.883 | 27.566 |
| 21 | EH38E1861287-enhD | chr17 | 38109004 | 38109312 | 772.83 | 19.565 | 80.125 | 106.9 | 13.049 | 18.433 |
| 22 | EH38E1664636-prom | chr13 | 31191678 | 31192027 | 131.075 | 14.132 | −113.8 | 255.5 | 97.095 | 236.51 |
| 23 | EH38E1861481-prom | chr17 | 38278710 | 38278967 | 763.254 | 357.604 | −10.88 | 67.1 | 25.254 | 6.124 |
| 24 | EH38E1874882-enhP | chr17 | 57696626 | 57696819 | 754.843 | −12.157 | −71.26 | 170.5 | −15.04 | 141.89 |
| 25 | EH38E2652215-prom | chr8 | 1.02E+08 | 1.02E+08 | 550.646 | 163.659 | −182.7 | 728.5 | 97.82 | 141.52 |
| 26 | EH38E1860841-enhP | chr17 | 37617462 | 37617812 | 723.7 | −2.682 | −20.56 | 80.52 | −18.68 | 9.468 |
| 27 | EH38E1858182-enhP | chr17 | 33414861 | 33415029 | 709.37 | 0 | −5.458 | 27.29 | −11.77 | 19.125 |
| 28 | EH38E1941416-prom | chr19 | 14629224 | 14629572 | 117.142 | −128.43 | 94.825 | 461.8 | 109.46 | 253.25 |
| 29 | EH38E1868861-enhP | chr17 | 47439042 | 47439237 | 679.906 | 11.821 | −31.13 | 7.302 | 26.298 | 49.18 |
| 30 | EH38E1932207-prom | chr19 | 2269237 | 2269586 | 176.245 | −103.49 | 37.733 | 375.3 | 70.377 | 195.31 |
| 31 | EH38E1861091-prom | chr17 | 37886655 | 37886998 | 677.268 | 399.395 | 29.409 | 78.19 | 38.404 | 8.806 |
| 32 | EH38E2129483-enhD | chr20 | 62273226 | 62273575 | 245.827 | 63.466 | 108.41 | 136.4 | 63.925 | 79.783 |
| 33 | EH38E2166336-prom | chr22 | 41842724 | 41843064 | 137.673 | −42.1 | −42.52 | 280.7 | −14.87 | 241.38 |
| 34 | EH38E2319390-prom | chr4 | 1.1E+08 | 1.1E+08 | 656.284 | −12.764 | −39.49 | 54.87 | −21.84 | 69.763 |
| 35 | EH38E1858085-prom | chr17 | 33307314 | 33307662 | 654.703 | 26.485 | −19.77 | 82.39 | −17.39 | 59.769 |
| 36 | EH38E1861103-enhP | chr17 | 37895696 | 37895880 | 645.411 | 9.939 | −4.465 | 29.51 | 51.571 | 25.951 |
| 37 | EH38E1944144-prom | chr19 | 18392447 | 18392786 | 62.525 | −98.347 | 20.028 | 628.2 | 149.23 | 210.52 |
| 38 | EH38E1861288-enhD | chr17 | 38109331 | 38109636 | 621.561 | −2.494 | 11.601 | 36.59 | 59.563 | 2.054 |
| 39 | EH38E1874884-prom | chr17 | 57697117 | 57697280 | 617.117 | 20.434 | −15.58 | 190 | 157.55 | 87.406 |
| 40 | EH38E1861047-prom | chr17 | 37844057 | 37844406 | 601.229 | 520.527 | 16.273 | 97.86 | −1.442 | 56.975 |
| 41 | EH38E1891725-prom | chr17 | 79008604 | 79008953 | 164.342 | −38.614 | −134.9 | 599.4 | 47.503 | 37.242 |
| 42 | EH38E1871876-enhP | chr17 | 53498585 | 53498739 | 599.211 | 17.371 | −1.522 | 12.1 | 0 | 36.649 |
| 43 | EH38E1860907-enhD | chr17 | 37729728 | 37730069 | 596.059 | 2.55 | 1.04 | 0.099 | 2.81 | 19.69 |
| 44 | EH38E1858183-enhP | chr17 | 33415059 | 33415332 | 591.483 | −0.805 | −5.903 | 10.54 | 0 | −1.635 |
| 45 | EH38E1861497-enhP | chr17 | 38296950 | 38297130 | 590.658 | 284.604 | −10.39 | 71.88 | 41.818 | 33.816 |
| 46 | EH38E1861100-prom | chr17 | 37894378 | 37894690 | 569.527 | 1.21 | −9.949 | 88.92 | −35.97 | 34.389 |
| 47 | EH38E1861440-prom | chr17 | 38256831 | 38257171 | 566.259 | 446.334 | −19.9 | 138.4 | −51 | 39.107 |
| 48 | EH38E1860985-prom | chr17 | 37793184 | 37793526 | 559.68 | 461.139 | −22.74 | 41.28 | 45.189 | 27.102 |
| 49 | EH38E2653972-enhP | chr8 | 1.04E+08 | 1.04E+08 | 559.017 | 123.229 | −65.89 | 217.1 | 14.33 | 224.44 |
| 50 | EH38E1860834-prom | chr17 | 37607424 | 37607772 | 557.384 | 7.215 | −10.58 | 30.68 | 22.378 | 36.24 |
| 51 | EH38E1992467-prom | chr2 | 43453701 | 43454045 | −5.457 | −54.074 | 8.659 | 308.6 | 32.211 | 87.567 |
| 52 | EH38E2318434-enhP | chr4 | 1.09E+08 | 1.09E+08 | 553.775 | 10.033 | 8.074 | 32.63 | 2.27 | 21.783 |
| 53 | EH38E2730450-enhD | chr9 | 1.32E+08 | 1.32E+08 | −23.566 | −1.246 | 28.64 | 110.2 | 28.524 | 101.56 |
| 54 | EH38E1664553-enhP | chr13 | 31039746 | 31039942 | −52.055 | 24.782 | 44.897 | 153 | 20.38 | 133.94 |
| 55 | EH38E1870485-prom | chr17 | 48943720 | 48944053 | 542.031 | −22.146 | −69.38 | 425.6 | 120.27 | 123.46 |
| 56 | EH38E1861525-enhP | chr17 | 38334264 | 38334419 | 402.085 | 541.91 | −6.054 | 21.66 | 71.035 | 34.689 |
| 57 | EH38E1857914-enhP | chr17 | 32908605 | 32908868 | 0 | 537.49 | 3.355 | 2.037 | 15.528 | 2.433 |
| 58 | EH38E2083049-prom | chr2 | 2.33E+08 | 2.33E+08 | 170.106 | −93.168 | −136.5 | 468.7 | 34.628 | 155.18 |
| 59 | EH38E2386096-prom | chr5 | 79551779 | 79552129 | 164.481 | −3.812 | −29.53 | 229.6 | 118.49 | 305 |
| 60 | EH38E1438750-rom | chr1 | 2.45E+08 | 2.45E+08 | 80.447 | 12.932 | −76.33 | 284.2 | 37.227 | 65.557 |
| 61 | EH38E2648741-prom | chr8 | 95961516 | 95961865 | 168.652 | 19.976 | 31.629 | 528.8 | 10.545 | 63.954 |
| 62 | EH38E1383711-enhP | chr1 | 1.51E+08 | 1.51E+08 | 80.512 | −51.093 | −109.2 | 290.3 | 79.335 | 134.76 |
| 63 | EH38E1653874-enhP | chr12 | 1.25E+08 | 1.25E+08 | 63.971 | −32.363 | −5.734 | 232.8 | 114.94 | 122.25 |
| 64 | EH38E1861120-enhD | chr17 | 37910538 | 37910888 | 426.335 | 519.74 | −5.036 | 0.415 | 18.982 | 42.63 |
| 65 | EH38E1886296-enhP | chr17 | 73781075 | 73781249 | 64.638 | −29.741 | −56.35 | 298.7 | −16.9 | 18.504 |
| 66 | EH38E1857911-prom | chr17 | 32907743 | 32907969 | −7.496 | 518.896 | −8.52 | −8.78 | 1.24 | −20.678 |
| 67 | EH38E1426891-prom | chr1 | 2.26E+08 | 2.26E+08 | 127.849 | 16.251 | −50.32 | 514.3 | 30.633 | 27.77 |
| 68 | EH38E1862833-prom | chr17 | 39844834 | 39845182 | −57.756 | −88.912 | −124.6 | 508.5 | −15.6 | 91.175 |
| 69 | EH38E1861437-enhP | chr17 | 38255328 | 38255676 | 507.347 | 396.656 | 10.983 | 186 | 34.929 | 0.082 |
| 70 | EH38E2163976-enhP | chr22 | 39096862 | 39097210 | 305.167 | −11.8 | 67.723 | 168.9 | 132.23 | 103.88 |
| 71 | EH38E2318435-prom | chr4 | 1.09E+08 | 1.09E+08 | 502.157 | 21.715 | −0.186 | 61 | 34.707 | 58.228 |
| 72 | EH38E1427455-prom | chr1 | 2.26E+08 | 2.26E+08 | 13.313 | −5.915 | −1.821 | 500.3 | 60.688 | 127.24 |
| 73 | EH38E1403139-prom | chr1 | 1.82E+08 | 1.82E+08 | 45.218 | −48.569 | −31.56 | 497.7 | −17.32 | 21.007 |
| 74 | EH38E2652396-prom | chr8 | 1.02E+08 | 1.02E+08 | 381.259 | 98.899 | −148.9 | 469 | 142.43 | 68.262 |
| 75 | EH38E2652655-prom | chr8 | 1.02E+08 | 1.02E+08 | 495.845 | 139.851 | −156.6 | 372.2 | 91.766 | 115.51 |
| 76 | EH38E1861060-enhP | chr17 | 37856804 | 37857149 | 392.263 | 495.121 | 12.128 | 10.74 | −16.14 | 33.239 |
| 77 | EH38E1699709-enhD | chr13 | 1.14E+08 | 1.14E+08 | 79.514 | 3.416 | 33.185 | 490.8 | 15.744 | −6.083 |
| 78 | EH38E2652214-prom | chr8 | 1.02E+08 | 1.02E+08 | 356.531 | 61.248 | −135 | 283.4 | 15.337 | 98.002 |
| 79 | EH38E1931948-prom | chr19 | 2051156 | 2051506 | 149.907 | −21.394 | −245.7 | 350.8 | 25.819 | 254.78 |
| 80 | EH38E1854193-prom | chr17 | 27055676 | 27055855 | 484.243 | −1.111 | −89.12 | 116.5 | 51.851 | 62.262 |
| 81 | EH38E2064069-enhP | chr2 | 1.98E+08 | 1.98E+08 | 106.954 | 1.384 | −69.93 | 184.9 | 84.151 | 230.35 |
| 82 | EH38E1860953-enhP | chr17 | 37773895 | 37774236 | 482.793 | 78.674 | 33.921 | 22.78 | −2.061 | 23.69 |
| 83 | EH38E2653974-prom | chr8 | 1.04E+08 | 1.04E+08 | 358.727 | 112.355 | −59.35 | 231.8 | 74.218 | 201.28 |
| 84 | EH38E1858201-prom | chr17 | 33446624 | 33446946 | 476.853 | −1.931 | −1.561 | −3.33 | 20.268 | 16.018 |
| 85 | EH38E1334665-enhP | chr1 | 32403646 | 32403800 | 140.478 | −16.405 | −73.01 | 29.6 | 134.37 | 94.866 |
| 86 | EH38E1854819-prom | chr17 | 27717464 | 27717693 | 473.173 | −2.242 | −14.92 | 66 | 17.23 | 54.461 |
| 87 | EH38E1432244-enhD | chr1 | 2.33E+08 | 2.33E+08 | 16.273 | 24.179 | 66.558 | 133.3 | 37.858 | 92.396 |
| 88 | EH38E2531601-prom | chr7 | 5570137 | 5570487 | 62.285 | −57.966 | −78.47 | 268.6 | −63.46 | 131.39 |
| 89 | EH38E1861313-enhP | chr17 | 38137367 | 38137564 | 463.224 | 10.412 | 4.153 | 20.63 | 7.113 | 17.056 |
| 90 | EH38E1860968-prom | chr17 | 37783418 | 37783578 | 461.059 | 186.161 | −16.85 | −16.8 | 1.36 | −9.725 |
| 91 | EH38E1545216-prom | chr11 | 65264862 | 65265210 | −6.378 | −62.197 | −21.24 | 129.6 | 15.202 | 5.443 |
| 92 | EH38E1698219-prom | chr13 | 1.11E+08 | 1.11E+08 | 91.428 | 66.868 | −77.32 | 453.7 | 53.384 | 103.96 |
| 93 | EH38E2730196-prom | chr9 | 1.32E+08 | 1.32E+08 | 78.252 | 11.918 | −43.31 | 93.93 | 10.833 | 152.1 |
| 94 | EH38E1861033-enhD | chr17 | 37831159 | 37831471 | 451.768 | 337.581 | 15.156 | −25.2 | 7.004 | 3.836 |
| 95 | EH38E2680430-enhD | chr9 | 6681608 | 6681759 | 1.467 | 52.672 | −18.47 | 83.85 | −11.43 | 35.903 |
| 96 | EH38E2690173-prom | chr9 | 33025003 | 33025351 | 154.17 | −11.983 | −61.78 | 449.2 | 99.618 | 239.86 |
| 97 | EH38E2164817-prom | chr22 | 39916421 | 39916584 | 88.265 | −18.356 | −33.51 | 149.9 | −30.86 | 69.786 |
| 98 | EH38E1857909-enhP | chr17 | 32906622 | 32906966 | −14.238 | 447.633 | −5.686 | −4.55 | 0 | −0.024 |
| 99 | EH38E1664551-enhP | chr13 | 31038722 | 31039047 | −11.495 | 17.37 | −60.76 | 197.7 | −31.33 | 79.844 |
| 100 | EH38E1860954-enhP | chr17 | 37774255 | 37774423 | 444.359 | 157.689 | 34.154 | −17.7 | −31.05 | 22.801 |

| | | | | | | | # w/ | # | |
|---|---|---|---|---|---|---|---|---|---|
| | | | Tumor_{—} | Gene_{—} | Mult. | SEACR_{—} | SEACR_{—} | Cancer | Encoded |
| Rank | Stomach | All | Type | name | hits | peaks | peaks | pubs | Protein |
| 1 | 43.414 | 2069.971 | Br | RFFL | | Br Co Li | 4 | 8 | Ring Finger And |
| | | | | | | Re | | | FYVE Like Domain |
| | | | | | | | | | Containing F3 |
| | | | | | | | | | Ubiquitin |
| | | | | | | | | | Protein Ligase |
| 2 | 63.973 | 1815.186 | Br | | RFFL | Br Co Li | 4 | | |
| | | | | | | Re | | | |
| 3 | 104.752 | 1353.096 | Br | MSL1 | | Br Li Re | 3 | 12 | Male-Specific |
| | | | | | | | | | Lethal-1 |
| | | | | | | | | | Homolog 1 |
| 4 | 278.05 | 1261.804 | Br | LIG3 | | Br Co Li | 5 | 108 | DNA |
| | | | | | | Re St | | | ligase 3 |
| 5 | 86.838 | 1221.496 | Br | | MSL1 | Br Li Lu | 4 | | |
| | | | | | | Re | | | |
| 6 | 102.963 | 1049.48 | Br | ORMDL3 | | Br Co Li | 3 | 32 | ORMDL |
| | | | | | | | | | Sphingolipid |
| | | | | | | | | | Biosynthesis |
| | | | | | | | | | Regulator 3 |
| 7 | 1045.35 | 1045.347 | St | HSP90AA1 | | St Br | 2 | 328 | Heat Shock |
| | | | | | | | | | Protein 90 |
| | | | | | | | | | Alpha Family |
| | | | | | | | | | Class A |
| | | | | | | | | | Member 1 |
| 8 | 22.483 | 1039.974 | Br | | RFFL | Br Co Li | 4 | | |
| | | | | | | Re | | | |
| 9 | 99.547 | 1027.798 | Br | CDK12 | | Br St | 2 | 418 | Cyclin- |
| | | | | | | | | | Dependent |
| | | | | | | | | | Kinase 12 |
| 10 | 21.831 | 983.622 | Br | | MSL1 | Br Li Lu | 4 | | |
| | | | | | | Re | | | |
| 11 | −35.444 | 965.776 | Br | MMD | | Br Co Li | 4 | 246 | Monocyte To |
| | | | | | | Re | | | Macrophage |
| | | | | | | | | | Differentiation |
| | | | | | | | | | Associated |
| 12 | 49.863 | 964.408 | Br | | ORMDL3 | Br Co Li | 3 | | |
| 13 | 53.29 | 949.349 | Br | CASC3 | | Br Co Li | 3 | 18 | Cancer |
| | | | | | | | | | Susceptibility |
| | | | | | | | | | Candidate |
| | | | | | | | | | Gene 3 Protein |
| 14 | 36.215 | 899.533 | Br | SEC24B | | Br Li Re | 3 | 7 | SEC24 |
| | | | | | | | | | Homolog B, |
| | | | | | | | | | COPII Coat |
| | | | | | | | | | Complex |
| | | | | | | | | | Component |
| 15 | 69.741 | 892.606 | Br | | MMD | Br Co Li | 4 | | |
| | | | | | | Re | | | |
| 16 | 96.608 | 869.45 | Br | | MSL1 | Br Li Re | 3 | | |
| 17 | 92.947 | 849.809 | Br | ZNF652 | | Br Co Li | 5 | 25 | Zinc Finger |
| | | | | | | Re St | | | Protein 652 |
| 18 | 133.189 | 815.99 | Co | ERBB2 | | Br Co St | 4 | 35,121 | Erb-B2 |
| | | | | | | Re | | | Receptor |
| | | | | | | | | | Tyrosine |
| | | | | | | | | | Kinase 2 |
| 19 | 139.158 | 808.318 | Br | | ERBB2 | Br Co St | 4 | | |
| | | | | | | Re | | | |
| 20 | 214.455 | 801.658 | Br | | ZNF652 | Br Co Li | 6 | | |
| | | | | | | Lu Re St | | | |
| 21 | 44.025 | 772.83 | Br | GSDMA | | Br Li | 2 | 55 | Gasdermin A |
| 22 | 770.142 | 770.142 | St | HMGB1 | | Br Li Re | 4 | 2,543 | High-Mobility |
| | | | | | | St | | | Group Box 2 |
| 23 | 45.997 | 763.254 | Br | | MSL1 | Br Li Lu | 4 | | |
| | | | | | | Re | | | |
| 24 | 96.585 | 754.843 | Br | CLTC | | Br Li Lu | 5 | 90 | Clathrin |
| | | | | | | Re St | | | Heavy Chain |
| 25 | 694.568 | 728.516 | Li | PABPC1 | | Br Li Lu | 5 | 121 | Poly(A) Binding |
| | | | | | | Re St | | | Protein |
| | | | | | | | | | Cytoplasmic 1 |
| 26 | 53.494 | 723.7 | Br | | CDK12 | Br Co Li | 3 | | |
| 27 | 0 | 709.37 | Br | | RFFL | Br Co Li | 4 | | |
| | | | | | | Re | | | |
| 28 | 695.594 | 695.594 | St | DNAJB1 | | St | 1 | 155 | DnaJ Heat |
| | | | | | | | | | Shock Protein |
| | | | | | | | | | Family (Hsp40) |
| | | | | | | | | | Member B1 |
| 29 | 108.253 | 679.906 | Br | | ZNF652 | Br Co Li | 5 | | |
| | | | | | | Re St | | | |
| 30 | 678.916 | 678.916 | St | OAZ1 | | Br Li Lu | 4 | 36 | Ornithine |
| | | | | | | St | | | Decarboxylase |
| | | | | | | | | | Antizyme 1 |
| 31 | 9.182 | 677.268 | Br | MIEN1 | | Br Co Li | 3 | 47 | Migration |
| | | | | | | | | | And Invasion |
| | | | | | | | | | Enhancer 1 |
| 32 | 667.94 | 667.94 | St | STMN3 | | Br Re St | 3 | 22 | Stathmin 3 |
| 33 | 663.515 | 663.515 | St | TOB2 | | Br Re St | 3 | 20 | Transducer |
| | | | | | | | | | Of ERBB2. 2 |
| 34 | 105.787 | 656.284 | Br | | SEC24B | Br Li Re | 3 | | |
| 35 | 83.809 | 654.703 | Br | | LIG3 | Br Co Li | 5 | | |
| | | | | | | Re St | | | |
| 36 | 108.039 | 645.411 | Br | GRB7 | | Br Co Li | 3 | 233 | Growth Factor |
| | | | | | | | | | Receptor-Bound |
| | | | | | | | | | Protein 7 |
| 37 | 620.219 | 628.231 | Li | JUND | | St | 1 | 488 | JUND |
| | | | | | | | | | protooncogene |
| 38 | 76.33 | 621.561 | Br | | GSDMA | Br Li | 2 | | |
| 39 | 192.457 | 617.117 | Br | | CLTC | Br Li Lu | 5 | | |
| | | | | | | Re St | | | |
| 40 | 52.742 | 601.229 | Br | PGAP3 | | Br Co Li | 3 | 22 | Post-GPI |
| | | | | | | | | | Attachment |
| | | | | | | | | | To Proteins |
| | | | | | | | | | Phospholipase 3 |
| 41 | 124.61 | 599.441 | Li | BAIAP2 | | Br Lu Re | 4 | 43 | BAR/IMD |
| | | | | | | St | | | Domain Containing |
| | | | | | | | | | Adaptor Protein 2 |
| 42 | 35.339 | 599.211 | Br | | MMD | Br Co Li | 4 | | |
| | | | | | | Re | | | |
| 43 | 13.743 | 596.059 | Br | PPP1R1B | | Br Co Li | 4 | 63 | Protein |
| | | | | | | Re | | | Phosphatase 1 |
| | | | | | | | | | Regulatory |
| | | | | | | | | | Inhibitor |
| 44 | 14.951 | 591.483 | Br | | RFFL | Br Co Li | 4 | | Subunit 1B |
| | | | | | | Re | | | |
| 45 | 105.169 | 590.658 | Br | | CASC3 | Br Li | 2 | | |
| 46 | 65.896 | 569.527 | Br | | GRB7 | Br Co Li | 3 | | |
| 47 | 46.908 | 566.259 | Br | NR1D1 | | Li | 1 | 113 | Nuclear Receptor |
| | | | | | | | | | Subfamily 1 |
| | | | | | | | | | Group D Member 1 |
| 48 | 52.922 | 559.68 | Br | STARD3 | | Br Co Li | 3 | 62 | StAR Related |
| | | | | | | | | | Lipid Transfer |
| | | | | | | | | | Domain |
| | | | | | | | | | Containing 3 |
| 49 | 549.192 | 559.017 | Br | AZIN1 | | Br Li Lu | 5 | 47 | Antizyme |
| | | | | | | Re St | | | Inhibitor 1 |
| 50 | 44.309 | 557.384 | Bt | MED1 | | Br | 1 | 378 | Mediator |
| | | | | | | | | | Complex |
| | | | | | | | | | Subunit 1 |
| 51 | 555.82 | 555.82 | St | ZFP36L2 | | Re St | 2 | 46 | ZFP36 Ring |
| | | | | | | | | | Finger Protein |
| | | | | | | | | | Like 2 |
| 52 | 30.78 | 553.775 | Br | PAPSS1 | | Br Co Li | 5 | 17 | 3′-Phospho- |
| | | | | | | Re St | | | adenosine in |
| | | | | | | | | | Phosphosulfate |
| | | | | | | | | | Synthase 1 |
| 53 | 552.386 | 552.386 | St | LINC01503 | | Li Lu Re | 4 | 30 | Long Intergenic |
| | | | | | | St | | | Non-Protein |
| | | | | | | | | | Coding RNA 1503 |
| 54 | 546.651 | 546.651 | St | | HMGB1 | Br Li Re | 4 | | |
| | | | | | | St | | | |
| 55 | 245.059 | 542.031 | Br | TOB1 | | Br Lu Re | 4 | 95 | Transducer |
| | | | | | | St | | | Of ERBB2. 1 |
| 56 | 22.015 | 541.91 | Co | RAPGEFL1 | | Br Co | 2 | 6 | Rap Guanine |
| | | | | | | | | | Nucleotide |
| | | | | | | | | | Exchange |
| | | | | | | | | | Factor Like 1 |
| 57 | 0 | 537.49 | Co | TMEM132E | | Co | 1 | 7 | Transmembrane |
| | | | | | | | | | Protein 132E |
| 58 | 533.142 | 533.142 | St | PTMA | | Br St | 2 | 64 | Prothymosine |
| | | | | | | | | | Alpha |
| 59 | 531.443 | 531.443 | St | SERINC5 | | Br Li Lu | 5 | 21 | Serine |
| | | | | | | Re St | | | Incorporator 5 |
| 60 | 530.334 | 530.334 | St | HNRNPU | | Br Li Lu | 5 | 65 | Heterogeneous |
| | | | | | | Re St | | | Nuclear |
| | | | | | | | | | Ribonucleo- |
| | | | | | | | | | protein U |
| 61 | 249.581 | 528.843 | Li | NDUFAF6 | | Br Li Re | 4 | 7 | NADH:Ubiquinone |
| | | | | | | St | | | Oxidoreductase |
| | | | | | | | | | Complex Assembly |
| | | | | | | | | | Factor 6 |
| 62 | 523.553 | 523.553 | St | MCL1 | | Br Li Lu | 4 | 5,250 | Myeloid Cell |
| | | | | | | St | | | Leukemia |
| | | | | | | | | | Sequence 1 |
| 63 | 521.746 | 521.746 | St | UBC | | Li Re St | 3 | 2,504 | Ubiquitin C |
| 64 | 139.209 | 519.74 | Co | IKZF3 | | Li Re St | 3 | 0 | IKAROS Family |
| | | | | | | | | | Zinc Finger 3 |
| 65 | 519.256 | 519.256 | St | UNK | | Li Re St | 3 | 0 | Unk Zinc Finger |
| 66 | 0 | 518.896 | Co | | TMEM132E | Co | 1 | | |
| 67 | 211.126 | 514.303 | Li | LBR | | Br Co Li | 5 | 183 | Lamin B Receptor |
| | | | | | | Re St | | | |
| 68 | 342.176 | 508.536 | Li | EIF1 | | St | 1 | 22 | Eukaryotic |
| | | | | | | | | | Translation |
| | | | | | | | | | Initiation |
| | | | | | | | | | Factor 1 |
| 69 | 70.682 | 507.347 | Br | | NR1D1 | Li Lu | 2 | | |
| 70 | 502.607 | 502.607 | St | JOSD1 | | Br Li Re | 4 | 12 | Josephine |
| | | | | | | St | | | Domain |
| | | | | | | | | | Containing 1 |
| 71 | 124.61 | 502.157 | Br | | PAPSS1 | Br Co Li | 5 | | |
| | | | | | | Re St | | | |
| 72 | 470.49 | 500.27 | Li | H3-3A | | Lu Re St | 3 | 120 | H3.3 Histone A |
| 73 | 304.078 | 497.682 | Li | GLUL | | Br Li Re | 4 | 81 | Glutamate- |
| | | | | | | St | | | Ammonia Ligase |
| 74 | 496.088 | 496.088 | St | YWHAZ | | Br Li Lu | 4 | 253 | Tyrosine 3/ |
| | | | | | | St | | | Tryptophan 5 - |
| | | | | | | | | | Monooxygenase |
| | | | | | | | | | Activation |
| | | | | | | | | | Protein |
| 75 | 388.454 | 495.845 | Br | ZNF706 | | Br Li Lu | 5 | 5 | Zinc Finger |
| | | | | | | Re St | | | Protein 706 |
| 76 | 6.122 | 495.121 | Co | | ERBB2 | Br Co Re | 4 | | |
| | | | | | | St | | | |
| 77 | 39.892 | 490.816 | Li | PROZ | | Br Co | 2 | 6 | Protein Z, |
| | | | | | | | | | Vitamin K |
| | | | | | | | | | Dependent Plasma |
| | | | | | | | | | Glycoprotein |
| 78 | 486.912 | 486.912 | St | | PABPC1 | Br Li Lu | 5 | | |
| | | | | | | Re St | | | |
| 79 | 485.249 | 485.249 | St | MKNK2 | | Br | 1 | 98 | MAPK Interacting |
| | | | | | | | | | Serine/Threonine |
| | | | | | | | | | Kinase 2 |
| 80 | 195.016 | 484.243 | Br | NEK8 | | Br | 1 | 23 | NIMA Related |
| | | | | | | | | | Kinase 8 |
| 81 | 483.098 | 483.098 | St | HSPD1 | | Br Li Lu | 5 | 159 | Heat Shock |
| | | | | | | Re St | | | Protein Family D |
| | | | | | | | | | (HSP60) Member 1 |
| 82 | 16.904 | 482.793 | Br | | PPP1R1B | Br Co | 2 | | |
| 83 | 480.495 | 480.495 | St | | AZIN1 | Br Li Lu | 5 | | |
| | | | | | | Re St | | | |
| 84 | −3.707 | 476.853 | Br | NR037714.1 | | Br | 1 | 0 | — |
| 85 | 476.096 | 476.096 | St | PTP4A2 | | Br Li Lu | 5 | 44 | Protein Tyrosine |
| | | | | | | Re St | | | Phosphatase 4A2 |
| 86 | 148.862 | 473.173 | Br | MIR4523 | | Br Co Li | 4 | 0 | MicroRNA 4523 |
| | | | | | | Re | | | |
| 87 | 465.668 | 465.668 | St | SIPA1L2 | | Co Li Re | 4 | 4 | Signal Induced |
| | | | | | | St | | | Proliferation |
| | | | | | | | | | Associated |
| | | | | | | | | | 1 Like 2 |
| 88 | 463.326 | 463.326 | St | ACTB | | St | 1 | 44 | Actin Beta |
| 89 | −17.211 | 463.224 | Br | PSMD3 | | Br Li | 2 | 22 | Proteasome |
| | | | | | | | | | 26S Subunit, |
| | | | | | | | | | Non-ATPase 3 |
| 90 | 0.713 | 461.059 | Br | | PPP1R1B | Br Co | 2 | | |
| 91 | 455.405 | 455.405 | St | MALAT1 | | Li Lu Re | 4 | 1,317 | Metastasis |
| | | | | | | St | | | Associated Lung |
| | | | | | | | | | Adenocarcinoma |
| | | | | | | | | | Transcript 1 |
| 92 | 387.12 | 453.671 | Li | ING1 | | Br Li Lu | 4 | 218 | Inhibitor of |
| | | | | | | St | | | Growth Family 1 |
| 93 | 452.049 | 452.049 | St | IER5L | | Br Li Lu | 5 | 4 | Immediate Early |
| | | | | | | Re St | | | Response 5 Like |
| 94 | −17.444 | 451.768 | Br | | PGAP3 | Br Co | 2 | | |
| 95 | 451.143 | 451.143 | St | GLDC | | Co Li Re | 4 | 57 | Glycine |
| | | | | | | St | | | Decarboxylase |
| 96 | 365.39 | 449.164 | Li | DNAJA1 | | Br Re St | 3 | 61 | DnaJ |
| | | | | | | | | | Heat Shock |
| | | | | | | | | | Protein Family |
| | | | | | | | | | (Hsp40) Member B2 |
| 97 | 448.045 | 448.045 | St | ATF4 | | Br Li Lu | 5 | 1,384 | Activating |
| | | | | | | Re St | | | Transcription |
| | | | | | | | | | Factor 4 |
| 98 | −6.997 | 447.633 | Co | | TMEM132E | Co | 1 | | |
| 99 | 446.24 | 446.24 | St | | HMGB1 | Br Li Lu | 5 | | |
| | | | | | | Re St | | | |
| 100 | 16.78 | 444.359 | Br | | PPP1R1B | Br Co | 2 | | |

### Example 5. FFPE-CUTACs v4

Chilling device (e.g.metal heat blocks on ice or cold packs in an ice cooler)
Pipettors (e.g. Rainin Classic Pipette 1 mL, 200 μL, 20 μL, and 10 μL)
Kimble Kontes Pellet Pestle Motor (DWK Life Sciences cat no. 749540-0000) Disposable pestles (Fisher cat. on. 12-141-364)
Disposable tips (e.g. Rainin 1 mL, 200 μL, 20 μL) Disposable centrifuge tubes for reagents (15 mL or 50 mL) Standard 1.5 mL and 2 mL microfuge tubes
0.5 ml maximum recovery PCR tubes (e.g. Fisher cat. no. 14-222-294)
4-10 micron section from a formaldehyde-fixed paraffin-embedded tissue block either as a curl or affixed to a charged glass slide
Strong magnet stand (e.g. Miltenyi Macsimag separator, cat. no. 130-092-168) Vortex mixer (e.g. VWR Vortex Genie)
Mini-centrifuge (e.g. VWR Model V)
PCR thermocycler (e.g. BioRad/MJ PTC-200) Safe Clear II (Fisher cat. no. 23-044192)
Bio-Mag Plus amine magnetic beads (48 mg/ml, Polysciences cat. no. 86001-10). Dilute 1:10 with 10 mM Tris pH8/1 mM EDTA for use.
Pierce glutathione magnetic beads (Fisher cat. no. 88822). Ethanol (Decon Labs, cat. no. 2716)
Distilled, deionized or RNAse-free H₂O (dH2O e.g., Promega, cat. no. P1197)
Roche Complete Protease Inhibitor EDTA-Free tablets (Sigma-Aldrich, cat. no. 5056489001) 1 M Tris-HCl pH 8.0
1 M Hydroxyethyl piperazineethanesulfonic acid pH 7.5 (HEPES (Na+); Sigma-Aldrich, cat. no. H3375) 5 M Sodium chloride (NaCl; Sigma-Aldrich, cat. no. S5150-1L)
2 M Spermidine (Sigma-Aldrich, cat. no. S0266) 10% Triton X-100 (Sigma-Aldrich, cat. no. X100)
0.5 M EDTA pH 8
10% Sodium azide (caution: toxic)
Antibody to an epitope of interest. Because in situ binding conditions are more like those for immunofluorescence (IF) than those for ChIP, we suggest choosing IF-tested antibodies if CUT&RUN/Tag-tested antibodies are not available CUTAC control antibody to RNA Polymerase II Phospho-Rpb1 CTD Serine-5 phosphate (PolIISSP, CST #13523 (D9N5I)).
Secondary antibody, e.g. guinea pig a-rabbit antibody (Antibodies online cat. no. ABIN101961) or rabbit a-mouse antibody (Abcam cat. no. ab46540)
Protein A/G-Tn5 (pAG-Tn5) fusion protein loaded with double-stranded adapters with 19mer Tn5 mosaic ends Epicypher cat. no. 15-1117.
Thermolabile Proteinase K (NEB P8111S)
1 M Magnesium Chloride (MgCl2; Sigma-Aldrich, cat. no. M8266-100G)
1 M [tris(hydroxymethyl)methylamino] propanesulfonic acid (TAPS) pH 8.5 (with NaOH) N,N-dimethylformamide (Sigma-Aldrich cat. no. D-8654-250 mL)
NEBNext 2X PCR Master mix (ME541L)
PCR primers: 10 μM stock solutions of i5 and i7 primers with unique barcodes [Buenrostro, J. D. et al. Nature 523:486 (2015)] in 10 mM Tris pH 8. Standard salt-free primers may be used. We do not recommend Nextera or NEBNext primers.
10% Sodium dodecyl sulfate (SDS; Sigma-Aldrich, cat. no. L4509)
SPRI paramagnetic beads (e.g. HighPrep PCR Cleanup Magbio Genomics cat. no. AC-60500)
   Reagent Setup (for up to 16 samples)

1. Cross-link reversal buffer Mix 8 ml 1 M Tris-HCl pH8.0, 2 ml dH20 and 4 μl 0.5 mM EDTA.

Rinse buffer (Option 1) Mix 1 mL 1 M HEPES pH 7.5 and 1.5 mL 5 M NaCl, and bring the final volume to 50 mL with dH20.

Triton-Wash buffer Mix 1 mL 1 M HEPES pH 7.5, 1.5 mL 5 M NaCl, 250 μl 10%

Triton-X100, 12.5 μl 2 M spermidine, bring the final volume to 50 mL with dH2O, and add 1 Roche Complete Protease Inhibitor EDTA-Free tablet. Store the buffer at 4° C. for up to 2 days.

Note: To completely prevent bacterial contamination during storage of Triton-wash buffer, add 0.2 mM EDTA or sodium azide to 0.02% (100 μl 10%->50 mL) or both. Handle sodium azide carefully and wear a mask when weighing it out.

Primary antibody solution Mix 17 μl RNA Polymerase II-Ser5p: (Cell Signaling Technologies (D9N51) mAb #13523)+423 μl Triton-Wash buffer (1:25).

Secondary antibody solution Mix 17 μl guinea pig anti-rabbit (Antibodies Online) with 423 μL Triton-Wash buffer (1:25).

Protein A (G)-Tn5 solution Mix 21 μl Protein A (G)-Tn5 (Epicypher cat. no. 15-1117) with 419 μL Triton-Wash buffer (1:20).

CUTAC-DMF Tagmentation buffer Mix 17.7 mL dH20, 4 mL N,N-dimethylformamide, 220 μl 1 M TAPS pH 8.5, and 110 μl 1 M MgCl2 (10 mM TAPS, 5 mM MgCl2, 20% DMF). Store the buffer at 4° C. for up to 1 week.

TAPS-EDTA wash buffer Mix 1 mL dH20, 10 μl 1 M TAPS pH 8.5, 0.4 μl 0.5 M EDTA (10 mM TAPS, 0.2 mM EDTA). Store at room temperature.

1% SDS/ProtK Release solution (For 16 samples) Mix 10 μl 10% SDS and 1 μl 1 M TAPS pH 8.5 in 79 μl dH2O. Just before use add 10 μL Thermolabile Proteinase K (NEB cat. no. P8111S).

5% Triton Mix 1 mL 10% Triton-X100+1 mLdH20. Store at room temperature.

### Option 1: On-Slide FFPE-CUTAC Deparaffinization in Hot Cross-Link Reversal Buffer

2. Place slides in cross-link reversal buffer in a slide holder that is filled to completely cover the slides. Place the holder in a water bath at 85-90° C. and incubate for at least an hour.

The paraffin will melt and float to the top. Remove slide holder to an ice-cold water bath to chill. Adding more solution to overfill will drain off any solid paraffin.

Note: Overnight 85-90° C. incubations in cross-link reversal buffer have yielded high-quality results similar to results using 1 hr incubations. Be sure that the FFPE sections are affixed to a charged glass slide to avoid tissue loss during incubations.

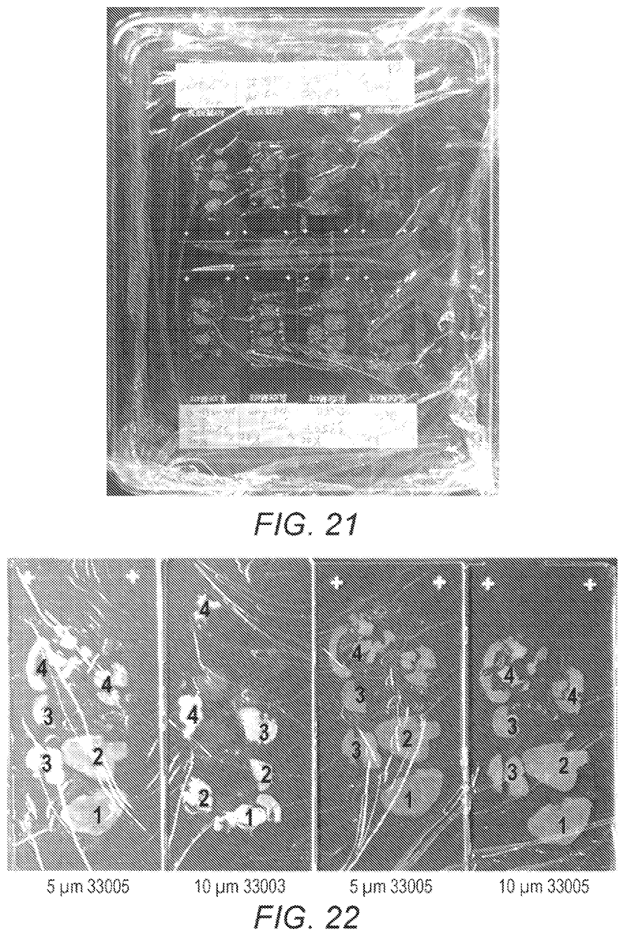

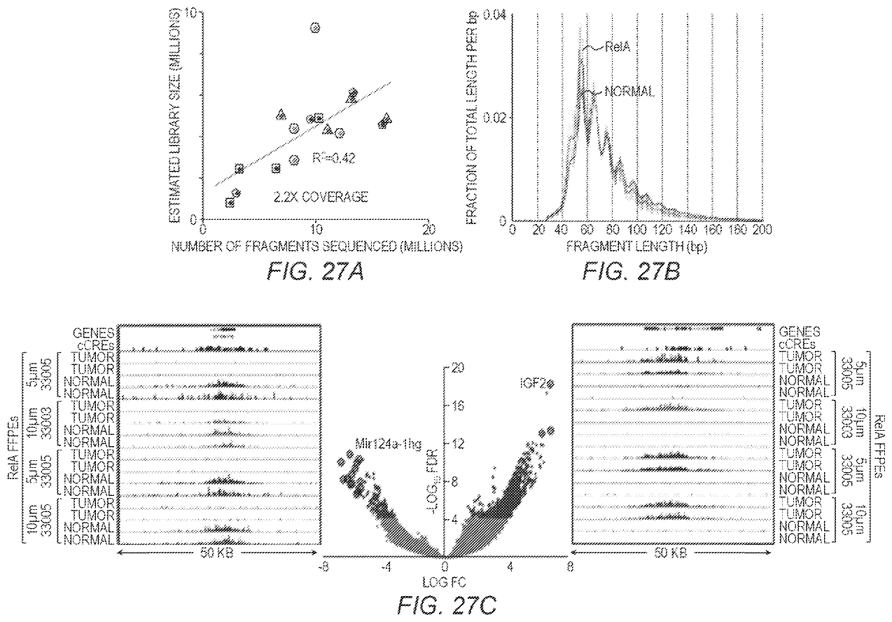

Note: The Option 1 protocol is for 16 samples but can be scaled up or down as needed. The example experiment shown in FIGS. 22, 23 and 27 beginning with dry FFPE slides through sequencing-ready purified DNA libraries was accomplished in one long day (~11 hours), but all of the steps can be lengthened with proper sealing to minimize evaporation. Overnight stopping points can be during any of the room temperature incubations by placing the plastic film-wrapped slides into a moist chamber and holding at 4-8° C.

3. Remove slides to Rinse Buffer in a slide holder.

4. For Option 1 (on-slide), continue with Step 5. For Option 2 (Magnetic Beads), skip to Step 22.

5. For each slide, remove from slide holder, wick off excess liquid from the glass surface with a Kimwipe (without touching the tissue) and place tissue-side up on a dark surface for visibility. Carefully pipette ~50 μl primary antibody solution over the tissue.

6. Cover the clear portion of the slide with a rectangle of plastic film (or a square for small tissue sections) using surface tension to spread the liquid, while excluding large bubbles and wrinkles. Place wrapped slides separated in a dry slide holder (FIG. 20) or in the rack of a staining dish, which can be used as a “moist chamber” (FIG. 25).

Note: Any bubbles over tissue can be pushed to a section of tissue-free glass.

Note: Other antibodies that work with this protocol are H3K27ac (Abcam #4729) and RNA Polymerase II Serine-2,5p (Cell Signaling Technologies CST (D1G3K) mAb #13546. Antibodies to histone methylations have failed, and unsatisfactory results have been obtained using an antibody to CTCF.

Note: Any plastic wrap will seal adequately, but we recommend food service film on a heavy 2000 foot roll (e.g. Reynolds 912) for ease of pulling out wrap with both hands. Some kitchen wraps (Saran and Glad) are not as smooth and will be more difficult to work with. Before removing slides from the Rinse Buffer, use a razor to cut plastic film rectangles slightly wider and longer than the clear portion of the slide.

7. Incubate at room temperature for at least 1 hr.

8. Remove plastic wrap and gently rinse slide by pipetting 1 mL Triton-Wash buffer dropwise over the top of the slide.

Option 1 (Continued): Incubation with Secondary Antibody (1.5 hr).

9. Wick off excess liquid with a Kimwipe and place tissue-side up on a dark surface. Carefully pipette ~50 μl secondary antibody solution over the tissue.

10. Cover the clear portion of the slide with a rectangle of plastic film using surface tension to spread the liquid, while omitting bubbles and folds. Place wrapped slides separated in a dry slide holder.

11. Incubate at room temperature for at least 1 hr.

12. Remove plastic wrap and gently rinse slide 1-2 times with 1 mL Triton-Wash buffer.

### Option 1 (Continued): Binding Protein a (G)-Tn5 Adapter Complex (1.5 hr)

13. Remove from slide holder and wick off excess liquid with a Kimwipe. Place tissue-side up on a dark surface. Carefully pipette ~50 μl pA (G)-Tn5 solution over the tissue.

14. Cover the clear portion of the slide with a rectangle of plastic film using surface tension to spread the liquid, while omitting bubbles and folds.

Note: When using other commercial sources of Protein A-Tn5 or Protein AG-Tn5 use the concentration recommended by the manufacturer for CUT&Tag. If using homemade fusion protein use the concentration recommended in the protocol for CUT&Tag, where the stock concentration may be higher (e. g., https://www.protocols.io/view/3xflag-patn5-protein-purification-and-meds-loading-j8nlke4e515r/v1).

15. Incubate at room temperature for at least 1 hr.

16. Remove plastic wrap and gently rinse slide 1-2 times with 1 mL Triton-Wash buffer. Drain on paper towel or Kimwipe and place in a slide holder filled with Triton-Wash buffer for 10 min. Drain and place in a slide holder with Triton-Wash buffer for 10 min.

17. Drain on paper towel and wick off excess liquid with a Kimwipe and place in a slide holder filled with 10 mM TAPS pH 8.5 for 10 min.

### Option 1 (Continued): Tagmentation and Dissection (1.5 hr)

18. Remove slides and drain on paper towel or Kimwipe and place in a slide holder containing cold Tagmentation buffer.

19. Incubate 1 hr in a water bath at 55° C.

20. Remove each slide to a slide holder containing TAPS-EDTA wash buffer to hold.

21. Remove slide from slide holder, drain and use a Kimwipe to remove excess liquid from the top surface. Dissect or scrape using a total of no more than 5 μL 1% SDS/Thermolabile Proteinase K solution per PCR tube. For larger tissue amounts, use more SDS/TLProtK solution and divide up into PCR tubes such that no more than 5 μL is deposited into each tube. To recover all tissue from the slide dice and scrape with a safety razor blade. Vortex and centrifuge to compact beads in the bottom of the tube and proceed to Fragment Release (Step 43).

Note: For dissection into a PCR tube, first add 2 μl to the tube, then 2 μl to the desired section of tissue using the pipette tip to spread the solution and loosen the tissue from the slide. Use a #3-5 jeweler's forceps and a scalpel or razor blade to scrape each section into a pile and deposit it into the PCR tube. A 1 μl aliquot of the solution can be used to remove the remaining tissue from the slide into the tube.

Note: Working quickly reduces the chance that tissue will dry out during dissection. However, we have not noticed any loss of data quality when tissue dries before being wetted with SDS/Proteinase K solution.

### Option 2: FFPE-CUTAC Using Beads: Deparaffinization in SafeCleanII.

22. FFPE slide or curl: Scrape all or part of a 5-10 μm FFPE slide (FIGS. 20, 22, 25) or a “curl” (FIG. 26) into a 1.5-2 ml tube (e.g., MCT-175-C). Add 320 μl Safe Clear II. Vortex, spin, and place in a 56° C. water bath for 3 min. Cool and centrifuge on full for 2 min.

Note: The Option 2 protocol is for 16 samples but can be scaled up or down as needed. Sequencing-ready purified DNA libraries can be obtained in one long day (~10 hours), but any of the 1 hr antibody or pAG-Tn5 incubations can be extended to a few hours at room temperature or at 4-8° C. overnight.

Note: Vortex hard to mix, but in some steps a “quick vortex” is used. With a touch mini-centrifuge, “spin on full” is just up to full speed then down, whereas “quick spin” is only to remove liquid from the cap and down from the sides.

Note: Curls are thin sections that are released from the microtome without being affixed to slides and either curl up to form a tight rod (10 μm) or fold (5 μm). Best permeabilization is obtained with 5 μm curls.

Note: Using more than half of a curl from a 10 micron section equivalent to the amount of tissue on the slides in FIGS. 20, 22, 25 might result in inhibition of PCR when using a one-tube protocol, and tube transfer before PCR is recommended.

23. Remove liquid avoiding the pellet. Quick spin and remove final liquid with low-bind pipette tip. Place in 37° C. heating block with caps open for 10 min.

24. Add 1 mL Cross-link reversal buffer and place in 900C water bath for 1 hr.

Note: 90° C. incubations can be extended for several hours or overnight without noticeable consequences. Likewise, room temperature incubations with affinity reagents can be extended up to overnight by performing at 4-8° C. Differences for longer room temperature or cold incubation times have not been noticed and times less than 1 hr, have not been tested which might be OK for shortening this protocol to fit into a single day.

25. When cool add 6 μl 1:10 Biomag beads (final concentration 4.8 mg/ml), vortex, then add 2.4 μl Pierce beads and vortex. Place on Rotator (or Nutator) for 10-20 min.

26. Do a quick spin and place on the magnet stand. When clear carefully remove ~850 μl with a 1 ml pipettor, quick spin followed by using a 200 μL low-bind pipette tip. Proceed immediately to antibody addition.

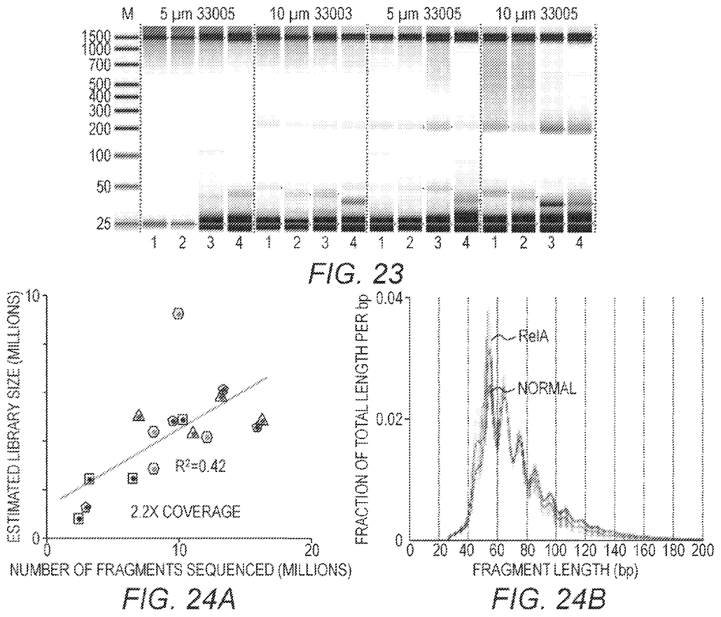

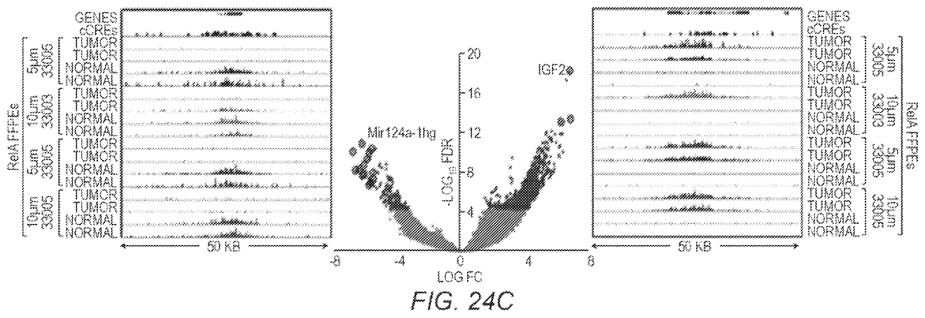

Note: Bio-Mag Plus amine magnetic beads are ~1.5 micron in diameter and have a rough hydrophilic surface that sticks weakly to deparaffinized tissue shards (FIG. 23). Pierce glutathione magnetic agarose beads are 10-40 micron but are inert and don't appear to stick, although they trap the tissue as they as they migrate in a magnetic field. In a magnetic field, the combination rapidly forms a tight pellet that is not disrupted by the pipette when decanting the supernatant.

Option 2 (Continued): Incubation with Primary Antibody

27. Resuspend beads in 100 μl primary antibody solution followed by vortexing.

Note: The protocol for FFPEs is similar to CUT&Tag-direct Version 4 and can be performed in parallel with native or lightly cross-linked nuclei or whole cells.

28. Incubate at least 1 hr on Rotator (or Nutator) at room temperature.

Option 2 (Continued): Incubation with Secondary Antibody

29. After a quick spin, place the tubes on the magnet stand to clear and carefully remove supernatant using a 200 μL low-bind pipette tip.

30. Resuspend beads in 100 μl secondary antibody solution followed by vortexing.

31. Incubate 1 hr on Rotator (or Nutator) at room temperature.

32. After a quick spin, place the tubes on the magnet stand to clear and carefully remove supernatant using a 200 μl low-bind pipette tip.

33. While on the magnet stand, slowly drip in 1 mL of Triton-Wash buffer. Carefully remove ~850 μl with a 1 mL pipettor and a quick spin followed by using a 200 μl low-bind pipette tip. Proceed immediately to the next step.

### Option 2 (Continued): Binding Protein a (G)-Tn5 Adapter Complex

34. Mix pAG-Tn5 pre-loaded adapter complex in Triton-Wash buffer following the manufacturer's instructions (e.g., 1:20 for EpiCypher pAG-Tn5).

Note: This protocol is not recommended for “homemade” pA-Tn5 following our purification protocol, because the contaminating E. coli DNA will be preferentially tagmented relative to the less accessible FFPE DNA under the stringent 55° C. conditions used here. If homemade pA-Tn5 is used, it is important to minimize the amount added (≤1:200).

35. Add 100 μl pA (G)-Tn5 mix followed by vortexing. Place the tubes on a Rotator or Nutator at room temperature for 1 hr.

36. After a quick spin, place the tubes on the magnet stand to clear and carefully remove supernatant using a 200 μl low-bind pipette tip.

37. While on the magnet stand, slowly drip in 1 mL Triton-Wash buffer. Carefully remove ~850 μl with a 1 mL pipettor and a quick spin followed by using a 200 μl low-bind pipette tip.

38. While on the magnet stand, slowly drip in 1 mL 10 mM TAPS pH8.5. Carefully remove ~850 μl with a 1 mL pipettor and a quick spin followed by using a 200 μl low-bind pipette tip. Proceed immediately to the next step.

### Option 2 (Continued): Tagmentation

39. Resuspend the bead/FFPE pellet in 200 μl CUTAC-DMF tagmentation solution (5 mM MgCl2, 10 mM TAPS, 20% DMF, 0.05% Triton-X100) while vortexing. Incubate at 55° C. for 1 hr in a thermocycler.

Note: N,N-dimethylformamide is a dehydrating compound resulting in improved tethered Tn5 accessibility and library yield. A 55° C. incubation used for FFPEs is the most stringent tested in Henikoff S, Henikoff J G, Kaya-Okur H S, Ahmad K. Efficient chromatin accessibility mapping in situ by nucleosome-tethered tagmentation. Elife. 2020 Nov. 16;9: e63274. doi: 10.7554/eLife.63274 (FIG. 3-figure supplement 2).

40. After a quick full centrifugation, place the tubes on a magnet stand and withdraw and discard the Tagmentation buffer supernatant using a 200 μl low-bind pipette tip.

41. While on the magnet stand, slowly drip in 1 mL TAPS-EDTA wash. Withdraw and discard the TAPS wash supernatant using a 200 μl low-bind pipette tip.

42. Add 10 μl 1% SDS/Thermolabile Proteinase K solution per PCR tube. Vortex, quick spin and proceed to Fragment Release (Step 43).

### Fragment Release and PCR

43. Incubate at 37° C. for 30 min and 58° C. for 30 min to release pA-Tn5 from the tagmented DNA. Open the tubes and add 36 μL 5% Triton-X100, close and incubate at 37° C. for 30 min on the cycler.

Note: Volumes here and below are calculated based on assuming that the tissue amount is equivalent to half that of a 10 micron FFPE slide or curl. Except for the sequencing primers, volumes may be scaled accordingly for different amounts of tissue.

44. Spin on full and remove supernatant to one or more PCR tubes for amplification.

Note: Previous versions of this protocol used the CUT&Tag-direct procedure without tube transfer, however, depending on the size and thickness of a curl or scrape, there is the risk of too much “gunk” inhibiting the PCR, and the PCR volume was increased from 50 μl to 100 μl. Owing to the very high efficiency of this protocol, where even 5% of a curl may be enough for a single library, either using the supernatant or splitting the bead slurry into multiple aliquots for PCR technical replicates is recommended.

45. Add 2 μl of 10 μM Universal or barcoded i5 primer+2 μl of 10 μM barcoded i7 primers, using a different barcode pair for each sample. Vortex on full and place tubes in the metal tube holder on ice.

Note: Indexed primers are described by Buenrostro, J. D. et al. Single-cell chromatin accessibility reveals principles of regulatory variation. Nature 523:486 (2015). Nextera or NEB primers are not recommended, which might not anneal efficiently using this PCR protocol.

46. Add 50 μl NEBnext (non-hot-start), vortex to mix, and perform a quick spin. Place the tubes in the thermocycler and proceed immediately with the PCR.

47. Begin the cycling program with a heated lid on the thermocycler:
Cycle 1:58° C. for 5 min (gap filling)
Cycle 2:72° C. for 5 min (gap filling)
Cycle 3:98° C. for 5 min
Cycle 4:98° C. for 10 sec
Cycle 5:63° C. for 30 sec
Cycle 6:72° C. for 1 min
   Repeat Cycles 4-6 11 times

### Hold at 8° C.

Note: CUT&Tag uses short 2-step 10 sec cycles to favor amplification of nucleosomal and smaller fragments. However, after cross-link reversal, DNA in FFPEs are small and PCR amplicon sizes <120 bp are recommended (Do and Dobrovic, Clin. Chem. 61 (1): 64-71 (2015)), which obviates the need to minimize the contribution of large DNA fragments. Insertion of a 1 min 72° C. extension and lengthening of the 63° C. annealing time from 10 sec to 30 sec results in better read-through of damaged DNA by Taq polymerase, resulting in a higher fraction of mappable reads than using the 2-step cycle favored for CUT&Tag and CUTAC.

Note: No more than 13 cycles are recommended, and fewer than 12 cycles may be optimal for larger curl samples. Extra PCR cycle reduce the complexity of the library. Post-PCR Clean-up (30 min)

48. After the PCR program ends, remove tubes from the thermocycler, vortex to resuspend, and add 130 μL of SPRI beads (ratio of 1.3 μL of SPRI beads to 1 μL of PCR product). Mix by pipetting up and down.

49. Let sit at room temperature 5-10 min.

50. Place on the magnet stand for a few minutes to allow the solution to clear.

51. Remove and discard the supernatant.

52. Keeping the tubes in the magnet stand, add 400 μL of 80% ethanol.

53. Completely remove and discard the supernatant.

54. Repeat Steps 52 and 53.

55. Perform a quick spin and remove the remaining supernatant, avoiding air drying the beads by proceeding immediately to the next step.

56. Remove from the magnet stand, add 22 μl 10 mM Tris-HCl pH 8, vortex and quick spin. Let sit for at least 5 min to elute the DNA.

57. Place on the magnet stand and allow to clear.

58. Remove the liquid to a fresh 1.5 mL tube with a pipette, avoiding transfer of beads.

### Tapestation Analysis and DNA Sequencing

59. Determine the size distribution and concentration of libraries by capillary electrophoresis using an Agilent 4200 TapeStation with D1000 reagents or equivalent.

Note: Quantification by Tapestation was used to estimate library concentration and dilute each library to 2 nM (or the concentration specified for Illumina library submission at the sequencing core that will process your sample) before pooling based on fragment molarity in the 175-500 bp range.

Note: Library samples from a single slide should be pooled using equal volumes to simplify comparisons between them. For direct comparisons between multiple slides processed in parallel using the same antibody, use equal volumes for all samples derived from them.

60. Mix barcoded libraries to achieve equal representation as desired aiming for a final concentration as recommended by the manufacturer. After mixing, perform an SPRI bead cleanup if needed to remove any residual PCR primers.

61. Perform paired-end Illumina sequencing on the barcoded libraries following the manufacturer's instructions.

Note: Paired-end 50×50 sequencing on an Illumina Next-Seq is currently used, obtaining ~400 million total mapped reads, or ~4 million per sample when there are 96 samples mixed to obtain approximately equal molarity.

### Data Processing and Analysis

62. Align paired-end reads to hg19 using Bowtie2 version 2.3.4.3 with options:--end-to-end--very-sensitive--no-unal--no-mixed--no-discordant--phred33-I 10-X 700. For mapping E. coli carry-over fragments, we also use the—-no-overlap--no-dovetail options to avoid possible cross-mapping of the experimental genome to that of the carry-over E. coli DNA that is used for calibration. Tracks are made as bedgraph files of normalized counts, which are the fraction of total counts at each basepair scaled by the size of the hg19 genome.

63. The CUT&Tag Data Processing and Analysis Tutorial on Protocols.io available at protocols.io/view/cut-amp-tag-data-processing-and-analysis-tutorial-e6nvw93x7gmk/v1 provides step-by-step guidance for mapping and analysis of CUT&Tag sequencing data. Most data analysis tools used for ChIP-seq data, such as bedtools, Picard and deepTools, can be used on CUT&Tag data. Analysis tools designed specifically for CUT&RUN/Tag data include the SEACR peak caller also available as a public web server and CUT&RUNTools.

The foregoing examples are illustrative of the present invention, and are not to be construed as limiting thereof. Although the invention has been described in detail with reference to preferred embodiments, variations and modifications exist within the scope and spirit of the invention as described and defined in the following claims.

1. An in situ method of mapping the location of a protein on chromatin in a cell from a formalin-fixed paraffin-embedded (FFPE) sample, comprising
a) treating the FFPE sample to remove the paraffin;
b) permeabilizing the sample;
c) contacting the sample with a first affinity reagent that specifically binds to a targeted chromatin protein, wherein the first affinity reagent is coupled to at least one transposome comprising: at least one transposase; and a transposon comprising: a first DNA molecule comprising a first transposase recognition site; and a second DNA molecule comprising a second transposase recognition site;
d) activating the at least one transposase under low ionic conditions, thereby cleaving and tagging chromatin DNA with the first and second DNA molecules;
e) excising the tagged DNA segment associated with the targeted chromatin protein; and
f) determining the nucleotide sequence of the excised tagged DNA segment, thereby mapping the genomic location of the targeted protein on chromatin.

2. A DNA-based in situ method for measuring transcription in a cell from a formalin-fixed paraffin-embedded (FFPE) sample, comprising:
a) treating the FFPE sample to remove the paraffin;
b) permeabilizing the sample;
c) contacting the sample with a first affinity reagent that specifically binds to a protein involved in transcription regulation, wherein the first affinity reagent is coupled to at least one transposome comprising: at least one transposase; and a transposon comprising: a first DNA molecule comprising a first transposase recognition site; and a second DNA molecule comprising a second transposase recognition site; activating the at least one transposase under low ionic conditions, thereby cleaving and tagging chromatin DNA with the first and second DNA molecules;
d) excising the tagged DNA segment associated with the protein involved in transcription regulation; and
e) determining the nucleotide sequence of the excised tagged DNA segment, thereby mapping transcriptional activity on chromatin.

3. The method of claim 1, further comprising using contaminating bacterial DNA as a calibration standard to normalize samples, optionally Rhodococcus DNA.

4. (canceled)

5. The method of claim 1, further comprising treating the sample with proteinase K prior to excising the tagged DNA segment, optionally a thermolabile proteinase K.

6. (canceled)

7. The method of claim 5, wherein excising the tagged DNA segment comprises adding a solution comprising about 1% SDS.

8. The method of claim 1, wherein the first affinity reagent is bound by a second affinity reagent.

9. The method of claim 8, wherein the second affinity reagent is bound by a third affinity reagent.

10. The method of claim 1, wherein the first, second, or third affinity reagent is directly or indirectly coupled to the at least one transposome.

11. (canceled)

12. The method of claim 1, wherein the first, second, and/or third affinity reagent is an antibody, an antibody-like molecule, a DARPin, an aptamer, a chromatin-binding protein, other specific binding molecule, or a functional antigen-binding domain thereof, optionally wherein the first, second, and/or third affinity reagent is an antibody to a phosphoform of the C-terminal domain of RNA polymerase II (RNAPII), such as RNAPII-Ser2, RNAPII-Ser5, RNAPII-Ser7, RNAPII-Ser2/5, or RNAPII-Ser5/7.

13. (canceled)

14. The method of claim 1, wherein the low ionic conditions are characterized by monovalent ionic concentration of less than about 10 mM.

15. The method of claim 1, wherein the transposome comprises a Tn5 transposase.

16. The method of claim 1, where the transposome comprises a barcode.

17. The method of claim 1, wherein the cell and/or a nucleus of the cell in the sample is permeabilized by contacting the cell with digitonin.

18. The method of claim 1, wherein at least one transposome comprises a fusion protein comprising a Tn5 transposase domain fused to a protein A domain, a protein G domain, or a protein A/G hybrid domain.

19-22. (canceled)

23. The method of claim 1, wherein the method is performed at single cell resolution.

24-25. (canceled)

26. The method of claim 1, wherein the sample, fragment, cell, or nucleus is bound to a solid support before or after permeabilization, optionally wherein the solid support is a bead, a slide, or a well (e.g. a microwell or nanowell), optionally an amine-functionalized bead, a lectin-coated bead, or a magnetic bead.

27-40. (canceled)

41. A method of monitoring a disease or disorder, the method comprising performing the method of claim 1 on samples obtained at two or more points in time from the same subject, and comparing an amount and/or the genomic location of the targeted protein on chromatin and/or the transcriptional activity on chromatin in each sample to a reference and/or to each other.

42. (canceled)

43. A method of diagnosing a disease or disorder in a subject, the method comprising performing the method of claim 1 on a sample from the subject, and diagnosing the subject as having the disease or disorder based on an amount and/or the genomic location of the targeted protein on chromatin and/or the transcriptional activity on chromatin to thereby diagnose the subject as having the disease or disorder.

44-47. (canceled)

48. A method of detecting presence and/or amount of a protein of interest on chromatin, the method comprising performing the method of claim 1, wherein the first affinity reagent that specifically binds to the targeted chromatin protein is specific for the protein of interest to thereby detect the presence and/or amount of the protein of interest on chromatin.

49-50. (canceled)

51. A composition comprising a deparaffinized and permeabilized FFPE sample containing an RNAPII specific affinity reagent or a chromatin protein specific affinity reagent that is linked directly or indirectly to a transposome in low ionic conditions.

52-53. (canceled)


SEQUENCE LISTING (2 sequences)

SEQ ID NO: 1 - DNA, 33 bp, synthetic construct
  source 1..33: mol_type = other DNA
agatcggaagagcacacgtctgaactccagtca

SEQ ID NO: 2 - DNA, 33 bp, synthetic construct
  source 1..33: mol_type = other DNA
agatcggaagagcgtcgtgtagggaaagagtgt